(19) United States
(12) Patent Application Publication
(10) Pub. No.: US 2013/0035515 A1
(43) Pub. Date: Feb. 7, 2013

(54) LIGNOCELLULOSIC HYDROLYSATES AS FEEDSTOCKS FOR ISOBUTANOL FERMENTATION

(71) Applicants: Ian David Dobson, London (GB); Arthur Leo Kruckeberg, Wilmington, DE (US)

(72) Inventors: Ian David Dobson, London (GB); Arthur Leo Kruckeberg, Wilmington, DE (US)

(73) Assignee: BUTAMAX(TM) ADVANCED BIOFUELS LLC, Wilmington, DE (US)

(21) Appl. No.: 13/525,032

(22) Filed: Jun. 15, 2012

Related U.S. Application Data
(60) Provisional application No. 61/498,209, filed on Jun. 17, 2011.

(51) Int. Cl. C12P 7/16; C12N 9/00; C07C 31/12

(52) U.S. Cl. 568/840; 435/160; 435/183

(57) ABSTRACT

The invention relates generally to the field of industrial microbiology and butanol production from sources of 5-carbon sugars such as lignocellulosic hydrolysates. More specifically, the invention relates to the use of an xylulose or xylulose-5-phosphate-producing enzyme and micro-aerobic or anaerobic conditions to increase butanol production from such sugars and recovery of said butanol through ins situ product recovery methods.

### CROSS-REFERENCE TO RELATED APPLICATIONS

This application is related to and claims the benefit of priority of U.S. Provisional Patent Application No. 61/498,209, filed Jun. 17, 2011. The contents of the referenced application are herein incorporated by reference in their entirety.

### REFERENCE TO SEQUENCE LISTING SUBMITTED ELECTRONICALLY

The content of the electronically submitted sequence listing in ASCII text file (Name: 20120615_CL5194USNP_SeqList.txt, Size: 1,164,851 bytes, and Date of Creation: Jun. 14, 2012) filed with the application is incorporated herein by reference in its entirety.

### FIELD OF THE INVENTION

The invention relates generally to the field of industrial microbiology and butanol production. More specifically, the invention relates to the use of microbes to convert 5-carbon sugars, including the 5-carbon sugars in hydrolysates of lignocellulosic biomass, to butanol as well as processes for recovering butanol from fermentation in the presence of mixed sugars.

### BACKGROUND OF THE INVENTION

Butanol is an important industrial chemical with a variety of applications, including use as a fuel additive, as a feedstock chemical in the plastics industry, and as a food-grade extractant in the food and flavor industry. Accordingly, there is a high demand for butanol, as well as for efficient and environmentally friendly production methods.

Production of butanol utilizing fermentation by microorganisms is one such environmentally friendly production method. A number of feedstocks can be used for such fermentative products. Among these are hydrolysates of lignocellulosic biomass, including corn cob, corn stover, switchgrass, bagasse, and wood waste. However, lignocellulosic hydrolysates also contain compounds that inhibit the growth and metabolism of the microorganisms used for their fermentation, and in particular, inhibit the growth and metabolism of microorganisms that are capable of producing butanol.

The present invention satisfies the current need to improve the production of butanol from such lignocellulosic hydrolysates by providing methods to efficiently convert 5-carbon sugars, obtainable from the lignocellulosic hydrolysates, to butanol as well as processes for recovering butanol from fermentation in the presence of mixed sugars.

### BRIEF SUMMARY OF THE INVENTION

The invention relates generally to the methods and compositions for butanol production from mixed sources of 5-carbon sugars and six-carbon sugars such as lignocellulosic hydrolysates and improved butanol production from said sugars with in situ product recovery methods. More specifically, the invention relates to the use of an xylulose or xylulose-5-phosphate-producing enzyme and micro-aerobic or anaerobic conditions to increase butanol production.

In some embodiments, a method for producing butanol comprises (a) providing a composition comprising (i) a microorganism capable of producing butanol and (ii) an enzyme or combination of enzymes capable of converting a 5-carbon sugar to xylulose or xylulose-5-phosphate; (b) contacting the composition with a carbon substrate comprising mixed sugars; and (c) culturing the microorganism under conditions of limited oxygen utilization, whereby butanol is produced.

### BRIEF DESCRIPTION OF THE DRAWINGS/FIGURES

The various embodiments of the invention can be more fully understood from the following detailed description, the figures, and the accompanying sequence descriptions, which form a part of this application.

FIG. 1: Growth on corn cob hydrolysate. Growth was monitored by packed cell volume using PCV tubes according to the manufacturer's instructions (TPP, Trasadingen, Switzerland). Results of triplicate flasks are shown. The isobutanologen (PNY1504, dashed lines) was grown in 0.5×LCH. The ethanologen (solid lines) was grown in 1×LCH.

FIG. 2: Consumption of glucose and production of isobutanol and glycerol by PNY1504. The results were measured over 148 hours, and metabolites were determined using HPLC.

FIG. 3: Consumption of glucose and production of ethanol and glycerol by CEN.PK113-7D. The results were measured over 148 hours, and metabolites were determined using HPLC.

FIG. 4: Fermentation of glucose to isobutanol by PNY1504. Profiles of glucose consumption (Glc), growth (Biomass, by Packed Cell Volume), and isobutanol production (Iso), in the presence (+AA; solid lines) or absence (−AA; dotted lines) of antimycin A are shown.

FIG. 5: Fermentation of xylose to isobutanol by PNY1504 in the presence of xylose isomerase. Profiles of xylose (Xyl) and xylulose (Xls) concentrations, growth (Biomass, by Packed Cell Volume), and isobutanol production (Iso), in the presence (+AA; solid lines) or absence (−AA; dotted lines) of antimycin A are shown.

FIG. 6: Profiles of glucose and xylose in lignocellulosic hydrolysate during fermentation to isobutanol. Cultures were either treated (solid line) or not treated (dotted lines) with antimycin A, and supplied (closed symbols) or not supplied (open symbols) with xylose isomerase.

FIG. 7: Isobutanol effective titers produced during fermentation of lignocellulosic hydrolysate. Cultures were either treated (solid line) or not (dotted lines) with antimycin A, and supplied (closed symbols) or not (open symbols) with xylose isomerase.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references' mentioned herein are incorporated by reference in their entireties for all purposes.

Although methods and materials similar or equivalent to those disclosed herein can be used in practice or testing of the present invention, suitable methods and materials are disclosed below. The materials, methods, and examples are illustrative only and are not intended to be limiting. Other features and advantages of the invention will be apparent from the detailed description and from the claims.

In order to further define this invention, the following terms, abbreviations and definitions are provided.

As used herein, the terms “comprises,” “comprising,” “includes,” “including,” “has,” “having,” “contains,” or “containing,” or any other variation thereof, are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences, of the element or component. Therefore “a” or “an” should be read to include one or at least one, and the singular word form of the element or component also includes the plural unless the number is obviously meant to be singular.

The term “invention” or. “present invention” as used herein is a non-limiting term and is not intended to refer to any single embodiment of the particular invention but encompasses all possible embodiments as disclosed in the application.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about,” the claims include equivalents to the quantities. In one embodiment, the term “about” means within 10% of the reported numerical value, preferably within 5% of the reported numerical value.

“Biomass” as used herein refers to a natural product containing a hydrolysable polysaccharide or carbohydrate that provides a fermentable sugar, including any cellulosic or lignocellulosic material and materials comprising cellulose, and optionally further comprising hemicellulose, lignin, starch, oligosaccharides, disaccharides, and/or monosaccharides. Biomass can also comprise additional components, such as protein and/or lipids. Biomass can be derived from a single source, or biomass can comprise a mixture derived from more than one source. For example, biomass can comprise a mixture of corn cobs and corn stover, or a mixture of grass stems and leaves. Biomass includes, but is not limited to, bioenergy crops, agricultural residues, municipal solid waste, industrial solid waste, sludge from paper manufacture, yard waste, wood, and forestry waste. Examples of biomass include, but are not limited to, corn grain, corn cobs, agricultural crop residues such as corn husks, corn stover, grasses, wheat, rye, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, municipal wastes and mixtures thereof.

“Butanol” as used herein refers with specificity to the butanol isomers 1-butanol (1-BuOH), 2-butanol (2-BuOH), isobutanol (iBuOH), and/or tert-butanol (t-BuOH), either individually or as mixtures thereof.

“Fermentable carbon source” as used herein means a carbon substrate from biomass capable of being metabolized by the microorganisms disclosed herein. Suitable fermentable carbon sources include, but are not limited to, monosaccharides, such as glucose or fructose, xylose and arabinose; disaccharides, such as maltose, lactose or sucrose; oligosaccharides; polysaccharides, such as starch or cellulose; one carbon substrates; and mixtures thereof.

“Feedstock” as used herein means a product containing a fermentable carbon source. Suitable feedstocks include, but are not limited to, rye, wheat, corn, cane, stover, switchgrass, bagasse and mixtures thereof.

“Fermentation broth” as used herein means the mixture of water, sugars (fermentable carbon sources), dissolved solids, microorganisms producing alcohol, product alcohol and all other constituents of the material held in the fermentation vessel in which product alcohol is being made by the reaction of sugars to alcohol, water and carbon dioxide (CO₂) by the microorganisms present. From time to time, as used herein the term “fermentation medium” and “fermented mixture” can be used synonymously with “fermentation broth”.

The term “carbon substrate” refers to a carbon source from biomass capable of being metabolized by the microorganisms and cells disclosed herein. Non-limiting examples of carbon substrates are provided herein and include, but are not limited to, monosaccharides, oligosaccharides, polysaccharides, ethanol, lactate, succinate, glycerol, carbon dioxide, methanol, glucose, fructose, sucrose, xylose, arabinose, dextrose, or mixtures thereof.

The term “effective titer” as used herein, refers to the total amount of a particular alcohol (e.g., butanol) produced by fermentation per liter of fermentation medium.

The term “separation” as used herein is synonymous with “recovery” and refers to removing a chemical compound from an initial mixture to obtain the compound in greater purity or at a higher concentration than the purity or concentration of the compound in the initial mixture.

The term “aqueous phase,” as used herein, refers to the aqueous phase of a biphasic mixture obtained by contacting a fermentation broth with a water-immiscible organic extractant. In an embodiment of a process described herein that includes fermentative extraction, the term “fermentation broth” then specifically refers to the aqueous phase in biphasic fermentative extraction.

The term “organic phase,” as used herein, refers to the non-aqueous phase of a biphasic mixture obtained by contacting a fermentation broth with a water-immiscible organic extractant.

The term “polynucleotide” is intended to encompass a singular nucleic acid as well as plural nucleic acids, and refers to a nucleic acid molecule or construct, e.g., messenger RNA (mRNA) or plasmid DNA (pDNA). A polynucleotide can contain the nucleotide sequence of the full-length cDNA sequence, or a fragment thereof, including the untranslated 5′ and 3′ sequences and the coding sequences. The polynucleotide can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. “Polynucleotide” embraces chemically, enzymatically, or metabolically modified forms.

A polynucleotide sequence can be referred to as “isolated,” in which it has been removed from its native environment. For example, a heterologous polynucleotide encoding a polypeptide or polypeptide fragment having enzymatic activity (e.g. the ability to convert a substrate to xylulose) contained in a vector is considered isolated for the purposes of the present invention. Further examples of an isolated polynucleotide include recombinant polynucleotides maintained in heterologous host cells or purified (partially or substantially) polynucleotides in solution. Isolated polynucleotides or nucleic acids according to the present invention further include such molecules produced synthetically. An isolated polynucleotide fragment in the form of a polymer of DNA can be comprised of one or more segments of cDNA, genomic DNA, or synthetic DNA.

The term “gene” refers to a nucleic acid fragment that is capable of being expressed as a specific protein, optionally including regulatory sequences preceding (5′ non-coding sequences) and following (3′ non-coding sequences) the coding sequence.

As used herein the term “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence that influence the transcription, RNA processing or stability, or translation of the associated coding sequence. Regulatory sequences can include promoters, translation leader sequences, introns, polyadenylation recognition sequences, RNA processing sites, effector binding sites and stem-loop structures.

As used herein, the term “polypeptide” is intended to encompass a singular “polypeptide” as well as plural “polypeptides” and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “dipeptides,” “tripeptides,” “oligopeptides,” “protein,” “amino acid chain,” or any other term used to refer to a chain or chains of two or more amino acids, are included within the definition of “polypeptide,” and the term “polypeptide” can be used instead of, or interchangeably with, any of these terms. A polypeptide can be derived from a natural biological source or produced by recombinant technology, but is not necessarily translated from a designated nucleic acid sequence. It can be generated in any manner, including by chemical synthesis.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposes of the invention, as are native or recombinant polypeptides which have been separated, fractionated, or partially or substantially purified by any suitable technique.

As used herein, “native” refers to the form of a polynucleotide, gene, or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene, or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene can include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide.

As used herein, the term “modification” refers to a change in a polynucleotide disclosed herein that results in altered activity of a polypeptide encoded by the polynucleotide, as well as a change in a polypeptide disclosed herein that results in altered activity of the polypeptide. Such changes can be made by methods well known in the art, including, but not limited to, deleting, mutating (e.g., spontaneous mutagenesis, random mutagenesis, mutagenesis caused by mutator genes, or transposon mutagenesis), substituting, inserting, altering the cellular location, altering the state of the polynucleotide or polypeptide (e.g., methylation, phosphorylation or ubiquitination), removing a cofactor, chemical modification, covalent modification, irradiation with UV or X-rays, homologous recombination, mitotic recombination, promoter replacement methods, and/or combinations thereof. Guidance in determining which nucleotides or amino acid residues can be modified, can be found by comparing the sequence of the particular polynucleotide or polypeptide with that of homologous polynucleotides or polypeptides, e.g., yeast or bacterial, and maximizing the number of modifications made in regions of high homology (conserved regions) or consensus sequences.

As used herein, the term “variant” refers to a polypeptide differing from a specifically recited polypeptide of the invention by amino acid insertions, deletions, mutations, and substitutions, created using, e.g., recombinant DNA techniques, such as mutagenesis. Guidance in determining which amino acid residues can be replaced, added, or deleted without abolishing activities of interest, can be found by comparing the sequence of the particular polypeptide with that of homologous polypeptides, e.g., yeast or bacterial, and minimizing the number of amino acid sequence changes made in regions of high homology (conserved regions) or by replacing amino acids with consensus sequences.

Alternatively, recombinant polynucleotide variants encoding these same or similar polypeptides can be synthesized or selected by making use of the “redundancy” in the genetic code. Various codon substitutions, such as silent changes which produce various restriction sites, can be introduced to optimize cloning into a plasmid or viral vector for expression. Mutations in the polynucleotide sequence can be reflected in the polypeptide or domains of other peptides added to the polypeptide to modify the properties of any part of the polypeptide.

Amino acid “substitutions” can be the result of replacing one amino acid with another amino acid having similar structural and/or chemical properties, i.e., conservative amino acid replacements, or they can be the result of replacing one amino acid with an amino acid having different structural and/or chemical properties, i.e., non-conservative amino acid replacements. “Conservative” amino acid substitutions can be made on the basis of similarity in polarity, charge, solubility, hydrophobicity, hydrophilicity, or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions can be made by selecting the differences in polarity, charge, solubility, hydrophobicity, hydrophilicity, or the amphipathic nature of any of these amino acids. “Insertions” or “deletions” can be within the range of variation as structurally or functionally tolerated by the recombinant proteins. The variation allowed can be experimentally determined by systematically making insertions, deletions, or substitutions of amino acids in a polypeptide molecule using recombinant DNA techniques and assaying the resulting recombinant variants for activity.

The term “promoter” refers to a DNA sequence capable of controlling the transcription of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different host cell types, or at different stages of development, or in response to different environmental or physiological conditions. Promoters which cause a gene to be expressed in most cell types at most times are commonly referred to as “constitutive promoters.” It is further recognized that since in most cases the exact boundaries of regulatory sequences have not been completely defined, DNA fragments of different lengths can have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “expression,” as used herein, refers to the transcription and stable accumulation of sense (mRNA) or antisense RNA derived from the nucleic acid fragment of the invention. Expression can also refer to translation of mRNA into a polypeptide.

The term “overexpression,” as used herein, refers to an increase in the level of nucleic acid or protein in a host cell. Thus, overexpression can result from increasing the level of transcription or translation of an endogenous sequence in a host cell or can result from the introduction of a heterologous sequence into a host cell. Overexpression can also result from increasing the stability of a nucleic acid or protein sequence.

The term “reduced activity and/or expression” of an endogenous protein such an enzyme can mean either a reduced specific catalytic activity of the protein (e.g. reduced activity) and/or decreased concentrations of the protein in the cell (e.g. reduced expression), while “deleted activity and/or expression” of an endogenous protein such an enzyme can mean either no or negligible specific catalytic activity of the enzyme (e.g. deleted activity) and/or no or negligible concentrations of the enzyme in the cell (e.g. deleted expression).

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid” and “vector” as used herein, refer to an extra-chromosomal element often carrying genes which are not part of the central metabolism of the cell, and usually in the form of circular double-stranded DNA molecules. Such elements can include be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and coding region for a selected gene product along with appropriate 3′ untranslated sequence into a cell.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the genes or coding regions of the nucleic acid molecules to reflect the typical codon usage of the host organism without altering the polypeptide encoded by the DNA. Such optimization includes replacing at least one, or more than one, or a significant number, of codons with one or more synonymous codons that are more frequently used in the genes of that organism. Codon-optimized coding regions can be designed by various methods known to those skilled in the art including software packages such as “synthetic gene designer” (http://phenotype.biosci.umbc.edu/codon/sgd/index.php).

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 1
The Standard Genetic Code

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATG Met | ACA Thr (T) | AAA Lys (K) | AGA Arg (R) |
| | (M) | ACG Thr (T) | AAG Lys (K) | AGG Arg (R) |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited Mar. 20, 2008), and these tables can be adapted in a number of ways. See Nakamura, Y., et al. Nucl. Acids Res. 28:292 (2000). Codon usage tables for yeast, calculated from GenBank Release 128.0 [15 Feb. 2002], are reproduced below as Table 2. This table uses mRNA nomenclature, and so instead of thymine (T) which is found in DNA, the tables use uracil (U) which is found in RNA. The Table has been adapted so that frequencies are calculated for each amino acid, rather than for all 64 codons.

TABLE 2
Codon Usage Table for Saccharomyces cerevisiae Genes

| | | | Frequency per |
|---|---|---|---|
| Amino Acid | Codon | Number | thousand |
| Phe | UUU | 170666 | 26.1 |
| Phe | UUC | 120510 | 18.4 |
| Leu | UUA | 170884 | 26.2 |
| Leu | UUG | 177573 | 27.2 |
| Leu | CUU | 80076 | 12.3 |
| Leu | CUC | 35545 | 5.4 |
| Leu | CUA | 87619 | 13.4 |
| Leu | CUG | 68494 | 10.5 |
| Ile | AUU | 196893 | 30.1 |
| Ile | AUC | 112176 | 17.2 |
| Ile | AUA | 116254 | 17.8 |
| Met | AUG | 136805 | 20.9 |
| Val | GUU | 144243 | 22.1 |
| Val | GUC | 76947 | 11.8 |
| Val | GUA | 76927 | 11.8 |
| Val | GUG | 70337 | 10.8 |
| Ser | UCU | 153557 | 23.5 |
| Ser | UCC | 92923 | 14.2 |
| Ser | UCA | 122028 | 18.7 |
| Ser | UCG | 55951 | 8.6 |
| Ser | AGU | 92466 | 14.2 |
| Ser | AGC | 63726 | 9.8 |
| Pro | CCU | 88263 | 13.5 |
| Pro | CCC | 44309 | 6.8 |
| Pro | CCA | 119641 | 18.3 |
| Pro | CCG | 34597 | 5.3 |
| Thr | ACU | 132522 | 20.3 |
| Thr | ACC | 83207 | 12.7 |
| Thr | ACA | 116084 | 17.8 |
| Thr | ACG | 52045 | 8.0 |
| Ala | GCU | 138358 | 21.2 |
| Ala | GCC | 82357 | 12.6 |
| Ala | GCA | 105910 | 16.2 |
| Ala | GCG | 40358 | 6.2 |
| Tyr | UAU | 122728 | 18.8 |
| Tyr | UAC | 96596 | 14.8 |
| His | CAU | 89007 | 13.6 |
| His | CAC | 50785 | 7.8 |
| Gln | CAA | 178251 | 27.3 |
| Gln | CAG | 79121 | 12.1 |
| Asn | AAU | 233124 | 35.7 |
| Asn | AAC | 162199 | 24.8 |
| Lys | AAA | 273618 | 41.9 |
| Lys | AAG | 201361 | 30.8 |
| Asp | GAU | 245641 | 37.6 |
| Asp | GAC | 132048 | 20.2 |
| Glu | GAA | 297944 | 45.6 |
| Glu | GAG | 125717 | 19.2 |
| Cys | UGU | 52903 | 8.1 |
| Cys | UGC | 31095 | 4.8 |
| Trp | UGG | 67789 | 10.4 |
| Arg | CGU | 41791 | 6.4 |
| Arg | CGC | 16993 | 2.6 |
| Arg | CGA | 19562 | 3.0 |
| Arg | CGG | 11351 | 1.7 |
| Arg | AGA | 139081 | 21.3 |
| Arg | AGG | 60289 | 9.2 |
| Gly | GGU | 156109 | 23.9 |
| Gly | GGC | 63903 | 9.8 |
| Gly | GGA | 71216 | 10.9 |
| Gly | GGG | 39359 | 6.0 |
| Stop | UAA | 6913 | 1.1 |
| Stop | UAG | 3312 | 0.5 |
| Stop | UGA | 4447 | 0.7 |

By utilizing this or similar tables, one of ordinary skill in the art can apply the frequencies to any given polypeptide sequence, and produce a nucleic acid fragment of a codon-optimized coding region which encodes the polypeptide, but which uses codons optimal for a given species.

Randomly assigning codons at an optimized frequency to encode a given polypeptide sequence can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences, e.g., the “backtranslation” function at http://www.entelechon.com/bioinformatics/backtranslation.php?lang=eng (visited Apr. 15, 2008) and the “backtranseq” function available at http://bioinfo.pbi.nrc.ca:8090/EMBOSS/index.html (visited Jul. 9, 2002). Constructing a rudimentary algorithm to assign codons based on a given frequency can also easily be accomplished with basic mathematical functions by one of ordinary skill in the art.

Codon-optimized coding regions can be designed by various methods known to those skilled in the art including software packages such as “synthetic gene designer” (http://phenotype.biosci.umbc.edu/codon/sgd/index.php).

A polynucleotide or nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementarity, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration can be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene-specific oligonucleotide probes comprising 20-30 contiguous nucleotides can be used in sequence-dependent methods of gene identification (e.g., Southern hybridization) and isolation (e.g., in situ hybridization of bacterial colonies or bacteriophage plaques). In addition, short oligonucleotides of 12-bases can be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular proteins. The skilled artisan, having the benefit of the sequences as reported herein, can now use all or a substantial portion of the disclosed sequences for purposes known to those skilled in this art. Accordingly, the instant invention comprises the complete sequences as provided herein, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those disclosed in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations can be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (disclosed by Higgins and Sharp, CABIOS. 5:151-153 (1989); Higgins, D. G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 55%, 60%, 65%, 70%, 75%, 80%, 85%, 90%, or 95%, or any integer percentage from 55% to 100% can be useful in describing the present invention, such as 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences.

“Sequence analysis software” can be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.](1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987). Additional methods used here are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

The genetic manipulations of cells disclosed herein can be performed using standard genetic techniques and screening and can be made in any cell that is suitable to genetic manipulation (Methods in Yeast Genetics, 2005, Cold Spring Harbor Laboratory Press, Cold Spring Harbor, N.Y., pp. 201-202).

### Sources of 5-Carbon Sugars

Hydrolysates of lignocellulosic biomass are a valuable feedstock for the production of biofuels that provide both 5- and 6-carbon sugars. However, these hydrolysates can contain compounds that are inhibitory to the growth and metabolism of microorganisms that are used to ferment the 5-carbon sugars. Thus, the amount of butanol that can be produced from lignocellulosic hydrolysates is limited because the 5-carbon sugars are not readily usable without certain genetic modifications and without some processing to ameliorate inhibitor activity. However, the methods described herein provide ways of increasing the yield of butanol from such lignocellulosic hydrolysates by allowing for the growth and metabolism of butanol-producing microorganisms and for the fermentation of 5-carbon sugars.

Biomass refers to any cellulosic or lignocellulosic material and includes materials comprising cellulose, and optionally further comprising hemicellulose, lignin, starch, oligosaccharides and/or monosaccharides. Biomass can also comprise additional components, such as protein and/or lipid. Biomass can be derived from a single source, or biomass can comprise a mixture derived from more than one source; for example, biomass can comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, agave, and mixtures thereof.

Fermentable sugars can be derived from such cellulosic or lignocellulosic biomass through processes of pretreatment and saccharification, as described, for example, in U.S. Pat. No. 7,781,191, which is herein incorporated by reference. By way of example, a relatively high concentration of biomass can be pretreated with a low concentration of ammonia relative to the dry weight of the biomass. Following the pretreatment, the biomass can be treated with a saccharification enzyme consortium to produce fermentable sugars. Thus, the pretreatment can comprise a) contacting biomass with an aqueous solution comprising ammonia to form a biomass-aqueous ammonia mixture, wherein the ammonia is present at a concentration at least sufficient to maintain alkaline pH of the biomass-aqueous ammonia mixture but wherein said ammonia is present at less than about 12 weight percent relative to dry weight of biomass, and further wherein the dry weight of biomass is at a high solids concentration of at least about 15 weight percent relative to the weight of the biomass-aqueous ammonia mixture; and b) contacting the product of step (a) with a saccharification enzyme consortium under suitable conditions, to produce fermentable sugars.

Ligriocellulosic hydrolysates and other sources of 5-carbon sugars can provide 5-carbon sugars and can provide 5-carbon sugars in combination with 6-carbon sugars or other carbon substrates which are suitable for fermentation. In some embodiments, the 5-carbon sugars are xylose. In some embodiments, the 5-carbon sugars are arabinose. In some embodiments, the 5-carbon sugars include both xylose and arabinose. The sources of 5-carbon sugars can also include other carbon substrates such as monosaccharides, polysaccharides, one-carbon substrates, two carbon substrates, and other carbon substrates. Hence it is contemplated that the source of carbon utilized in the present invention can encompass any number of carbons substrates in addition to the 5-carbon sugars.

In some embodiments, the lignocellulosic hydrolysate is present in the composition for fermentation at a particular concentration. For example, in some embodiments, the lignocellulosic hydrolysate is present at a concentration of at least about 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 110 g/L, 120 g/L, 130 g/L, 140 g/L, 150 g/L, 160 g/L, 170 g/L, 180 g/L, 190 g/L, or 200 g/L. In some embodiments, the lignocellulosic hydrolysate is present at a concentration of about 5-500 g/L, about 5-400 g/L, about 5-300 g/L, about 5-200 g/L, or about 5-150 g/L. In some embodiments, the lignocellulosic hydrolysate is present at a concentration of about 25-500 g/L, about 25-400 g/L, about 25-300 g/L, about 25-200 g/L, or about 25-150 g/L. In some embodiments, the lignocellulosic hydrolysate is present at a concentration of about 50-500 g/L, about 50-400 g/L, about 50-300 g/L, about 50-200 g/L, or about 50-150 g/L.

In addition, in some embodiments, the lignocellulosic hydrolysate is consumed at a particular rate. Thus, in some embodiments, asssuming 6 g/l cell mass like in corn and a TS level of 20% for straw gives C5 consumption at 0.44 g/l-h or a specific rate of 0.07 g C5/g cell hour.

In particular, 5-carbon sugars can be consumed from the lignocellulosic hydrolysate at a particular rate.

Production of Xylulose from Sources of 5-Carbon Sugars

Microorganisms that can be used according to the methods described herein can ferment xylulose via the pentose phosphate pathway. However, many sources of 5-carbon sugars, such a lignocellulosic hydrolysates, can contain 5-carbon sugars other than xylulose that cannot be directly fermented by the microorganisms. Therefore, the methods described herein provide enzymes that are capable of converting other 5-carbon sugars to D-xylulose and/or D-xylulose-5-P. For example, enzymes that can convert xylose or arabinose to xylulose are known to those of skill in the art. By way of example: xylose isomerase can convert xylose to D-xylulose; xylose reductase and xylitol dehydrogenase can convert xylose to D-xylulose; arabinose reductase, arabitol dehydrogenase, L-xylulose reductase, and xylitol dehydrogenase can convert arabinose to D-xylulose; arabinose isomerase, ribulokinase, and ribulose-phosphate-5-epimerase can convert arabinose to D-xylulose-5-P. In addition, aldose reductase, which can covert alditol to aldose is useful in converting arabinose and xylose into D-xylulose 5-P.

The enzyme or enzymes capable of converting other 5-carbon sugars to xylulose can be provided from an exogenous source or can be produced by recombinant microorganisms in the fermenting composition.

For example, xylulose-producing enzymes can be produced by any means known to those of skill in the art (including natural production, recombinant production and chemical synthesis), and a composition comprising the xylulose-producing enzymes can be added to butanol-producing microorganisms in order to ferment 5-carbon sugars. Xylulose-producing enzymes, such as xylose isomerase can be purchased from commercial sources, e.g., “Sweetzyme” produced by Novozyme.

Additionally, and/or alternatively, cells and/or microorganisms that express xylulose- and/or xylulose-5-P-producing enzymes can be added to the butanol-producing organisms in order to ferment 5-carbon sugars. The cells and/or microorganisms can be cells and/or microorganisms that convert 5-carbon sugars to xylulose and/or xylulose-5-P endogenously or can be cells and/or microorganisms that have been engineered to recombinantly produce xylulose- and/or xylulose-5-P-producing enzymes. Additionally, and/or alternatively, the butanol-producing microorganisms can be engineered to recombinantly produce xylulose- and/or D-xylulose-5-P-producing enzymes. Further, in the host cells of the invention, the expression of the araA, araB and araD enzymes, which provide for utilization of L-arabinose, combined with genetic modification that reduces unspecific aldose reductase activity, provide for efficient utilization of L-arabinose in the pentose-phosphate pathway (PPP). See e.g., U.S. Pat. No. 7,354,755, herein incorporated by reference. The genetic modification leading to the reduction of unspecific aldose reductase activity may be combined with any of the modifications increasing the flux of the pentose phosphate pathway and/or with any of the modifications increasing the specific xylulose kinase activity in the host cells as described herein. Thus, a host cell expressing araA, araB, and araD, comprising an additional genetic modification that reduces unspecific aldose reductase activity is specifically included in the invention. The genes expressing araA, araB and araD may be derived from E. coli or B. subtilis. Where the host cell is a yeast strain, in certain embodiments the yeast strain includes at least one arabinose transporter gene selected from the group consisting of GAL2, KmLAT1 and PgLAT2. The L-arabinose transporter with high affinity may be sourced from Kluyveromyces marxianus and Pichia guilliermondii (also known as Candida guilliermondii), respectively. Both Kluyveromyces marxianus and Pichia guilliermondii may be considered efficient utilizers of L-arabinose, which renders them a sources for cloning L-arabinose transporter genes. In certain embodiments the yeast strain further may overexpress a GAL2-encoded galactose permease. See also, U.S. Pat. No. 5,514,583, which is herein incorporated by reference. Other xylose utilizing strains include CP4(pZB5) (U.S. Pat. No. 5,514,583), ATCC31821/pZB5 (U.S. Pat. No. 6,566,107), 8b (US 20030162271; Mohagheghi et al., (2004) Biotechnol. Lett. 25; 321-325), and ZW658 (ATTCC #PTA-7858), which may be modified for butanol production from mixed sugars including xylose and glucose.

Thus, in order to improve butanol production, microorganisms can be engineered to express enzymes capable of producing xylulose and/or xylulose-5-P. The overall activity of xylulose- and/or xylulose-5-P-producing enzymes in a host cell can be increased by the introduction of heterologous nucleic acid and/or protein sequences or by mutation of endogenous nucleic acid and/or protein sequences. When a heterologous xylulose- and/or xylulose-5-P-producing enzyme gene or protein is introduced into a host cell, the enzymatic activity of the host cell is increased relative to the enzymatic activity in the absence of the heterologous nucleic acids or proteins. When an endogenous nucleic acid or protein is mutated in a host cell, the activity of the enzymes in the host cell is increased relative to the enzymatic activity in the absence of the mutation. In some embodiments, the rate of xylulose and/or xylulose-5-P production in a cell is increased relative to a wild-type yeast strain.

Xylulose- and/or D-xylulose-5-P-producing enzymes can be overexpressed individually or in combination in host strains. In some embodiments, xylose isomerase is overexpressed. In some embodiments, xylose reductase and xylitol dehydrogenase are overexpressed. In some embodiments, enzymes that produce xylulose and/or xylulose-5-P from arabinose are overexpressed. In some embodiments, xylose isomerase, xylose reductase, and xylitol dehydrogenase are overexpressed. In some embodiments, enzymes that convert xylose to xylulose and enzymes that convert arabinose to xylulose and/or xylulose-5-P are both overexpressed.

The introduction of xylulose- and/or xylulose-5-P-producing enzymes into a recombinant host cell can increase butanol production. In some embodiments of the methods described herein, a polynucleotide encoding a protein with the desired activity can be introduced into a cell using recombinant DNA technologies that are well known in the art. In some embodiments, the introduction of a polynucleotide encoding a protein with, for example, xylose isomerase, xylose reductase, or xylitol dehydrogenase activity results in improved isobutanol concentrations and increased specific isobutanol production rates.

Methods of making microorganisms that express xylulose-producing enzymes are known in the art. For example, International Publication No. WO 2009/109630, which is hereby incorporated by reference in its entirety, illustrates the production of pentose-sugar fermenting cells that express xylose isomerase. Additional examples of xylulose- and/or xylulose-5-P-producing enzyme genes and polypeptides that can be expressed in a host cell disclosed herein include, but are not limited to, those in Table 3 below, with sequences provided in attached Tables, herein incorporated by reference. Example xylose isomerase enzymes and source organisms for such polypeptides are disclosed in US20110318801A1. Examples of xylose isomerase and source organisms include, but are not limited those in Tables 4 and 5 below (e.g. SEQ ID NOs: 89-394) as well as SEQ ID NOs: 74, 75, and 395-399.

TABLE 3
Xylulose-Producing Enzymes.
Entire GenBank records are given in FASTA
format. Coding regions for the enzymes of
interest are given at the end of the header
line in parentheses

| | EC | |
|---|---|---|
| Enzyme | Number | SEQ ID NO |
| D-Xylulose | 1.1.1.9 | >gi\|3262\|emb\|X55392.1\| |
| reductase | | Pichia stipitis XYL2 gene for |
| | | xylitol dehydrogenase (319-1410) |
| | | TCTAGACCACCCTAAGTCGTCCCTATGTCGTATGTT |
| | | TGCCTCTACTACAAAGTTACTAGCAAATATCCGCAG |
| | | CAACAACAGCTGCCCTCTTCCAGCTTCTTAGTGTGT |
| | | TGGCCGAAAAGGCGCTTTCGGGCTCCAGCTTCTGTC |
| | | CTCTGCGGCTGCTGCACATAACGCGGGGACAATGA |
| | | CTTCTCCAGCTTTTATTATAAAAGGAGCCATCTCCT |
| | | CCAGGTGAAAAATTACGATCAACTTTTACTCTTTTC |
| | | CATTGTCTCTTGTGTATACTCACTTTAGTTTGTTTCA |
| | | ATCACCCCTAATACTCTTCACACAATTAAAATGACT |
| | | GCTAACCCTTCCTTGGTGTTGAACAAGATCGACGAC |
| | | ATTTCGTTCGAAACTTACGATGCCCCAGAAATCTCT |
| | | GAACCTACCGATGTCCTCGTCCAGGTCAAGAAAAC |
| | | CGGTATCTGTGGTTCCGACATCCACTTCTACGCCCA |
| | | TGGTAGAATCGGTAACTTCGTTTTGACCAAGCCAAT |
| | | GGTCTTGGGTCACGAATCCGCCGGTACTGTTGTCCA |
| | | GGTTGGTAAGGGTGTCACCTCTCTTAAGGTTGGTGA |
| | | CAACGTCGCTATCGAACCAGGTATTCCATCCAGATT |
| | | CTCCGACGAATACAAGAGCGGTCACTACAACTTGT |
| | | GTCCTCACATGGCCTTCGCCGCTACTCCTAACTCCA |
| | | AGGAAGGCGAACCAAACCCACCAGGTACCTTATGT |
| | | AAGTACTTCAAGTCGCCAGAAGACTTCTTGGTCAA |
| | | GTTGCCAGACCACGTCAGCTTGGAACTCGGTGCTCT |
| | | TGTTGAGCCATTGTCTGTTGGTGTCCACGCCTCCAA |
| | | GTTGGGTTCCGTTGCTTTCGGCGACTACGTTGCCGT |
| | | CTTTGGTGCTGGTCCTGTTGGTCTTTTGGCTGCTGCT |
| | | GTCGCCAAGACCTTCGGTGCTAAGGGTGTCATCGTC |
| | | GTTGACATTTTCGACAACAAGTTGAAGATGGCCAA |
| | | GGACATTGGTGCTGCTACTCACACCTTCAACTCCAA |
| | | GACCGGTGGTTCTGAAGAATTGATCAAGGCTTTCG |
| | | GTGGTAACGTGCCAAACGTCGTTTTGGAATGTACTG |
| | | GTGCTGAACCTTGTATCAAGTTGGGTGTTGACGCCA |
| | | TTGCCCCAGGTGGTCGTTTCGTTCAAGTTGGTAACG |
| | | CTGCTGGTCCAGTCAGCTTCCCAATCACCGTTTTCG |
| | | CCATGAAGGAATTGACTTTGTTCGGTTCTTTCAGAT |
| | | ACGGATTCAACGACTACAAGACTGCTGTTGGAATC |
| | | TTTGACACTAACTACCAAAACGGTAGAGAAAATGC |
| | | TCCAATTGACTTTGAACAATTGATCACCCACAGATA |
| | | CAAGTTCAAGGACGCTATTGAAGCCTACGACTTGG |
| | | TCAGAGCCGGTAAGGGTGCTGTCAAGTGTCTCATT |
| | | GACGGCCCTGAGTAAGTCAACCGCTTGGCTGGCCC |
| | | AAAGTGAACCAGAAACGAAAATGATTATCAAATAG |
| | | CTTTATAGACCTTTATCGAAATTTATGTAAACTAAT |
| | | AGAAAAGACAGTGTAGAAGTTATATGGTTGCATCA |
| | | CGTGAGTTTCTTGAATTCTTGAAAGTGAAGTCTTGG |
| | | TCGGAACAAACAAACAAAAAAATATTTTCAGCAAG |
| | | AGTTGATTTCTTTTCTGGAGATTTTGGTAATTGACA |
| | | GAGAACCCCTTTCTGCTATTGCCATCTAAACATCCT |
| | | TGAATAGAACTTTACTGGATGGCCGCCTAGTGTTGA |
| | | GTATATATTATCAACCAAAATCCTGTATATAGTCTC |
| | | TGAAAAATTTGACTATCCTAACTTAACAAAAGAGC |
| | | ACCATAATGCAAGCTCATAGTTCTTAGAGACACCA |
| | | ACTATACTTAGCCAAACAAAATGTCCTTGGCCTCTA |
| | | AAGAAGCATTCAGCAAGCTTCCCCAGAAGTTGCAC |
| | | AACTTCTTCATCAAGTTTACCCCCAGACCGTTTGCC |
| | | GAATATTCGGAAAAGCCTTCGACTATAGTGGATCC |
| | | (SEQ ID NO: 76) |
| L-Xylulose | 1.1.1.10 | >gi\|20378203\|gb\|AF375616.1\| |
| reductase | | Hypocrea jecorina L-xylulose |
| | | reductase mRNA, complete cds (85-885) |
| | | GCCTCGTCCGCCATCTCCCGTCTCACCAGTCGCTGT |
| | | CAATCAAGATTCATCACGAAATACTCCCCATCCTTT |
| | | GCATCGCCCATCATGCCTCAGCCTGTCCCCACCGCC |
| | | AACAGACTCCTTGATCTCTTCAGCTTGAAGGGCAA |
| | | GGTCGTCGTCGTCACCGGCGCTTCCGGCCCTCGAGG |
| | | CATGGGAATCGAAGCTGCCCGTGGCTGCGCCGAGA |
| | | TGGGCGCTGACCTCGCCATCACCTACTCGTCTCGCA |
| | | AGGAGGGCGCGGAGAAGAACGCCGAGGAATTGAC |
| | | CAAGGAATACGGCGTCAAAGTCAAGGTGTACAAGG |
| | | TCAACCAGAGCGACTACAACGATGTTGAGCGCTTT |
| | | GTGAACCAGGTCGTGTCTGACTTTGGCAAGATCGA |
| | | TGCCTTTATTGCCAACGCCGGAGCCACAGCTAATA |
| | | GCGGAGTTGTTGACGGCAGCGCCAGCGATTGGGAC |
| | | CATGTCATCCAGGTCGACCTGAGCGGCACCGCATA |
| | | CTGCGCAAAGGCTGTTGGCGCGCACTTCAAGAAGC |
| | | AGGGCCACGGCTCCCTTGTCATCACAGCTTCAATGT |
| | | CCGGCCACGTCGCAAACTATCCCCAGGAACAGACC |
| | | TCATACAACGTCGCCAAGGCCGGTTGCATCCATCTG |
| | | GCGCGGTCTCTGGCCAACGAGTGGCGTGATTTTGCC |
| | | CGCGTCAACAGCATTTCGCCCGGTTATATCGATACC |
| | | GGCCTGTCCGACTTCATCGACGAGAAGACGCAAGA |
| | | GCTGTGGAGGAGCATGATCCCCATGGGACGAAACG |
| | | GCGATGCCAAGGAGCTCAAGGGCGCGTATGTATAT |
| | | CTGGTCAGCGACGCTAGCTCGTACACGACGGGAGC |
| | | CGATATTGTGATTGACGGAGGTTACACTACACGAT |
| | | AAAGAAATAATGTATTGTTAGACTATAATCAATGT |
| | | GACGAACAAGATTTGTGATTAAGAAAAAAAAAAA |
| | | AAAAAAAAAAAAA |
| | | (SEQ ID NO: 77) |
| L-Arabitol | 1.1.1.12 | >gi\|15811374\|gb\|AF355628.1\| |
| 4- | | Hypocrea jecorina L- |
| dehydrogenase | | arabinitol 4-dehydrogenase (LAD1) |
| | | mRNA, complete cds (164-1297) |
| | | CCCAAGAAGGCCTGGAACAGAAGATCAAAAGCAG |
| | | AGAAGAGAGCGTATATAAGCATACATACACTCCCT |
| | | CTGCTCCGGTATTGTGGTTGATCTCCAAACGCGTCA |
| | | TCCCTCCCAACCCTCAAACGCCTTGTTCGCCGGAGA |
| | | CCGCGCGCATTCACAGCTCGCCATGTCGCCTTCCGC |
| | | AGTCGATGACGCTCCCAAGGCCACAGGGGCAGCCA |
| | | TCTCAGTCAAGCCCAACATTGGCGTCTTCACAAATC |
| | | CAAAACATGACCTCTGGATTAGCGAAGCTGAACCC |
| | | AGCGCCGATGCCGTCAAATCTGGCGCTGATCTGAA |
| | | GCCCGGCGAGGTGACCATTGCTGTCCGCAGCACTG |
| | | GTATCTGTGGTTCAGATGTCCATTTCTGGCACGCCG |
| | | GCTGCATTGGGCCCATGATCGTCGAGGGCGACCAC |
| | | ATCCTCGGCCACGAGTCTGCCGGCGAGGTCATCGC |
| | | CGTCCACCCGACTGTCAGTAGCCTCCAAATCGGCG |
| | | ATCGGGTTGCCATCGAGCCCAACATCATCTGCAAC |
| | | GCATGCGAGCCCTGCCTGACAGGTCGATACAACGG |
| | | CTGCGAAAAGGTCGAGTTCCTATCCACGCCGCCAG |
| | | TGCCCGGACTGCTGCGACGCTACGTCAACCACCCA |
| | | GCCGTTTGGTGCCACAAGATTGGCAACATGTCGTG |
| | | GGAGAACGGCGCGCTGCTGGAGCCCCTGAGCGTGG |
| | | CTCTGGCCGGCATGCAGAGGGCCAAGGTTCAGCTC |
| | | GGTGACCCCGTGCTGGTCTGCGGCGCTGGTCCGATT |
| | | GGATTGGTGTCAATGCTGTGCGCTGCTGCCGCCGGT |
| | | GCTTGCCCGCTTGTCATCACAGACATTTCAGAGAGC |
| | | CGTCTGGCGTTTGCAAAGGAGATCTGCCCCCGCGTC |
| | | ACCACGCACCGCATCGAGATTGGCAAGTCGGCTGA |
| | | GGAAACGGCCAAAAGCATCGTCAGCTCTTTTGGGG |
| | | GCGTCGAGCCAGCCGTGACCCTGGAGTGCACCGGT |
| | | GTGGAGAGCAGCATTGCAGCGGCCATCTGGGCCAG |
| | | CAAGTTTGGAGGAAAGGTCTTTGTGATCGGCGTCG |
| | | GCAAGAATGAAATCAGCATTCCCTTTATGAGGGCC |
| | | AGTGTACGCGAGGTCGATATCCAGCTGCAGTATCG |
| | | CTACAGCAACACCTGGCCTCGTGCCATCCGGCTCAT |
| | | CGAGAGCGGTGTCATCGATCTATCCAAATTTGTGAC |
| | | GCATCGCTTCCCGCTGGAGGATGCCGTCAAGGCAT |
| | | TTGAGACGTCAGCAGATCCCAAGAGCGGCGCCATT |
| | | AAGGTCATGATTCAGAGCCTGGATTGAGAGTGAGG |
| | | TGCTACCAGGTAGAGGTAGATAATAGATAGATGAT |
| | | GAAGATGGAAAGACTGCGGGCGCAAGAATCGGGC |
| | | GGATAGGGAGTTGGCTGTAATGGTTTGCAAAGCAT |
| | | AAAAAAAAAAAAAAAAAAAAA |
| | | (SEQ ID NO: 78) |
| Aldose | 1.1.1.21 | >gi\|3260\|emb\|X59465.1\| P.stipitis |
| reductase | | XYL1-gene for NAD(P)H-dependent |
| | | Xylose reductase (356-1312) |
| | | GATCCACAGACACTAATTGGTTCTACATTATTCGTG |
| | | TTCAGACACAAACCCCAGCGTTGGCGGTTTCTGTCT |
| | | GCGTTCCTCCAGCACCTTCTTGCTCAACCCCAGAAG |
| | | GTGCACACTGCAGACACACATACATACGAGAACCT |
| | | GGAACAAATATCGGTGTCGGTGACCGAAATGTGCA |
| | | AACCCAGACACGACTAATAAACCTGGCAGCTCCAA |
| | | TACCGCCGACAACAGGTGAGGTGACCGATGGGGTG |
| | | CCAATTAATGTCTGAAAATTGGGGTATATAAATAT |
| | | GGCGATTCTCCGGAGAATTTTTCAGTTTTCTTTTCAT |
| | | TTCTCCAGTATTCTTTTCTATACAACTATACTACAAT |
| | | GCCTTCTATTAAGTTGAACTCTGGTTACGACATGCC |
| | | AGCCGTCGGTTTCGGCTGTTGGAAAGTCGACGTCG |
| | | ACACCTGTTCTGAACAGATCTACCGTGCTATCAAGA |
| | | CCGGTTACAGATTGTTCGACGGTGCCGAAGATTAC |
| | | GCCAACGAAAAGTTAGTTGGTGCCGGTGTCAAGAA |
| | | GGCCATTGACGAAGGTATCGTCAAGCGTGAAGACT |
| | | TGTTCCTTACCTCCAAGTTGTGGAACAACTACCACC |
| | | ACCCAGACAACGTCGAAAAGGCCTTGAACAGAACC |
| | | CTTTCTGACTTGCAAGTTGACTACGTTGACTTGTTC |
| | | TTGATCCACTTCCCAGTCACCTTCAAGTTCGTTCCA |
| | | TTAGAAGAAAAGTACCCACCAGGATTCTACTGTGG |
| | | TAAGGGTGACAACTTCGACTACGAAGATGTTCCAA |
| | | TTTTAGAGACCTGGAAGGCTCTTGAAAAGTTGGTC |
| | | AAGGCCGGTAAGATCAGATCTATCGGTGTTTCTAA |
| | | CTTCCCAGGTGCTTTGCTCTTGGACTTGTTGAGAGG |
| | | TGCTACCATCAAGCCATCTGTCTTGCAAGTTGAACA |
| | | CCACCCATACTTGCAACAACCAAGATTGATCGAAT |
| | | TCGCTCAATCCCGTGGTATTGCTGTCACCGCTTACT |
| | | CTTCGTTCGGTCCTCAATCTTTCGTTGAATTGAACC |
| | | AAGGTAGAGCTTTGAACACTTCTCCATTGTTCGAGA |
| | | ACGAAACTATCAAGGCTATCGCTGCTAAGCACGGT |
| | | AAGTCTCCAGCTCAAGTCTTGTTGAGATGGTCTTCC |
| | | CAAAGAGGCATTGCCATCATTCCAAAGTCCAACAC |
| | | TGTCCCAAGATTGTTGGAAAACAAGGACGTCAACA |
| | | GCTTCGACTTGGACGAACAAGATTTCGCTGACATTG |
| | | CCAAGTTGGACATCAACTTGAGATTCAACGACCCA |
| | | TGGGACTGGGACAAGATTCCTATCTTCGTCTAAGA |
| | | AGGTTGCTTTATAGAGAGGAAATAAAACCTAATAT |
| | | ACATTGATTGTACATTTAAAATTGAATATTGTAGCT |
| | | AGCAGATTCGGAAATTTAAAATGGGAAGGTGATTC |
| | | TATCCGTACGAATGATCTCTATGTACATACACGTTG |
| | | AAGATAGCAGTACAGTAGACATCAAGTCTACAGAT |
| | | CATTAAACATATCTTAAATTGTAGAAAACTATAAA |
| | | CTTTTCAATTCAAACCATGTCTGCCAAGGAATCAAA |
| | | TGAGATTTTTTTCGCAGCCAAACTTGAATCCAAAAA |
| | | TAAAAAACGTCATTGTCTGAAACAACTCTATCTTAT |
| | | CTTTCACCTCATCAATTCATTGCATATCATAAAAGC |
| | | CTCCGATAGCATACAAAACTACTTCTGCATCATATC |
| | | TAAATCATAGTGCCATATTCAGTAACAATACCGGT |
| | | AAGAAACTTCTATTTTTTTAGTCTGCCTTAACGAGA |
| | | TGCAGATCGATGCAACGTAAGATCAAACCCCTCCA |
| | | GTTGTACAGTCAGTCATATAGTGAACACCGTACAA |
| | | TATGGTATCTACGTTCAAATAGACTCCAATACAGCT |
| | | GGTCTGCCCAAGTTGAGCAACTTTAATTTAGAGAC |
| | | AAAGTCGTCTCTGTTGATGTAGGCACCACACATTCT |
| | | TCTCTTGCCCGTGAACTCTGTTCTGGAGTGGAAACA |
| | | TCTCCAGTTGTCAAATATCAAACACTGACCAGGCTT |
| | | CAACTGGTAGAAGATTTCGTTTTCGG |
| | | (SEQ ID NO: 79) |
| Ribulokinase | 2.7.1.16 | >gi\|145303\|gb\|M15263.1\|ECOARAABD |
| | | E.coli araBAD operon encoding |
| | | L-ribulokinase, L-arabinose |
| | | isomerase, and L-ribulose |
| | | 5-phosphate 4-epimerase (120-1820) |
| | | CGTCACACTTTGCTATGCCATAGCATTTTTATCCAT |
| | | AAGATTAGCGGATCCTACCTGACGCTTTTTATCGCA |
| | | ACTCTCTACTGTTTCTCCATACCCGTTTTTTTGGATG |
| | | GAGTGAAACGATGGCGATTGCAATTGGCCTCGATT |
| | | TTGGCAGTGATTCTGTGCGAGCTTTGGCGGTGGACT |
| | | GCGCCAGCGGTGAAGAGATCGCCACCAGCGTAGAG |
| | | TGGTATCCCCGTTGGCAAAAAGGGCAATTTTGTGAT |
| | | GCCCCGAATAACCAGTTCCGTCATCATCCGCGTGAC |
| | | TACATTGAGTCAATGGAAGCGGCACTGAAAACCGT |
| | | GCTTGCAGAGCTTAGCGTCGAACAGCGCGCAGCTG |
| | | TGGTCGGGATTGGCGTTGACAGTACCGGCTCGACG |
| | | CCCGCACCGATTGATGCCGACGGTAACGTGCTGGC |
| | | GCTGCGCCCGGAGTTTGCCGAAAACCCGAACGCGA |
| | | TGTTCGTATTGTGGAAAGACCACACTGCGGTTGAA |
| | | AGAAGCGAAGAGATTACCCGTTTGTGCCACGCGCC |
| | | GGGCAATGTTGACTACTCCCGCTATATTGGCGGTAT |
| | | TTATTCCAGCGAATGGTTCTGGGCAAAAATCCTGCA |
| | | TGTGACTCGCCAGGACAGCGCCGTGGCGCAATCTG |
| | | CCGCATCGTGGATTGAGCTGTGCGACTGGGTGCCA |
| | | GCTCTGCTTTCCGGTACCACCCGCCCGCAGGATATT |
| | | CGTCGCGGACGTTGCAGCGCCGGGCATAAATCTCT |
| | | GTGGCACGAAAGCTGGGGCGGCTTGCCGCCAGCCA |
| | | GTTTCTTTGATGAGCTGGACCCGATCCTCAATCGCC |
| | | ATTTGCCTTCCCCGCTGTTCACTGACACCTGGACTG |
| | | CCGATATTCCGGTGGGCACCTTATGCCCGGAATGG |
| | | GCGCAGCGTCTCGGCCTGCCTGAAAGCGTGGTGAT |
| | | TTCCGGCGGCGCGTTTGACTGCCATATGGGCGCAGT |
| | | TGGCGCAGGCGCACAGCCTAACGCACTGGTAAAAG |
| | | TTATCGGTACTTCCACCTGCGACATTCTGATTGCCG |
| | | ACAAACAGAGCGTTGGCGAGCGGGCAGTTAAAGGT |
| | | ATTTGCGGTCAGGTTGATGGCAGCGTGGTGCCTGG |
| | | ATTTATCGGTCTGGAAGCAGGCCAATCGGCGTTTG |
| | | GTGATATCTACGCCTGGTTCGGTCGCGTACTCAGCT |
| | | GGCCGCTGGAACAGCTTGCCGCCCAGCATCCGGAA |
| | | CTGAAAGCGCAAATCAACGCCAGCCAGAAACAACT |
| | | GCTTCCGGCGCTGACCGAAGCATGGGCCAAAAATC |
| | | CGTCTCTGGATCACCTGCCGGTGGTGCTCGACTGGT |
| | | TTAACGGTCGTCGCTCGCCAAACGCTAACCAACGC |
| | | CTGAAAGGGGTGATTACCGATCTTAACCTCGCTACC |
| | | GACGCTCCGCTGCTGTTCGGCGGTTTGATTGCTGCC |
| | | ACCGCCTTTGGCGCACGCGCAATCATGGAGTGCTTT |
| | | ACCGATCAGGGGATCGCCGTCAATAACGTGATGGC |
| | | GCTGGGCGGCATCGCGCGGAAAAACCAAGTCATTA |
| | | TGCAGGCCTGCTGCGACGTGCTGAATCGCCCGCTG |
| | | CAAATTGTTGCCTCTGACCAGTGCTGTGCGCTCGGT |
| | | GCGGCGATTTTTGCTGCCGTCGCCGCGAAAGTGCA |
| | | CGCAGACATCCCATCAGCCCAGCAAAAAATGGCCA |
| | | GTGCGGTAGAGAAAACCCTGCAACCGCGCAGCGAA |
| | | CAGGCACAACGCTTTGAACAGCTTTATCGCCGCTAT |
| | | CAGCAATGGGCGATGAGCGCCGAACAACACTATCT |
| | | TCCAACTTCCGCCCCGGCACAGGCTGCCCAGGCCG |
| | | TTGCGACTCTATAAGGACACGATAATGACGATTTTT |
| | | GATAATTATGAAGTGTGGTTTGTCATTGGCAGCCAG |
| | | CATCTGTATGGCCCGGAAACCCTGCGTCAGGTCAC |
| | | CCAACATGCCGAGCACGTTGTTAATGCGCTGAATA |
| | | CGGAAGCGAAACTGCCCTGCAAACTGGTGTTGAAA |
| | | CCGCTGGGCACCACGCCGGATGAAATCACCGCTAT |
| | | TTGCCGCGACGCGAATTACGACGATCCGTGCGCTG |
| | | GTCTGGTGGTGTGGCTGCACACCTTCTCCCCGGCCA |
| | | AAATGTGGATCAACGGCCTGACCATGCTCAACAAA |
| | | CCGTTGCTGCAATTCCACACCCAGTTCAACGCGGCG |
| | | CTGCCGTGGGACAGTATCGATATGGACTTTATGAA |
| | | CCTGAACCAGACTGCACATGGCGGTCGCGAGTTCG |
| | | GCTTCATTGGCGCGCGTATGCGTCAGCAACATGCC |
| | | GTCGTTACCGGTCACTGGCAGGATAAACAAGCCCA |
| | | TGAGCGTATCGGCTCCTGGATGCGTCAGGCGGTTTC |
| | | TAAACAGGATACCCGTCATCTGAAAGTCTGCCGTTT |
| | | TGGCGATAACATGCGTGAAGTGGCGGTCACCGATG |
| | | GTGATAAAGTTGCCGCACAGATCAAGTTCGGTTTCT |
| | | CCGTCAATACCTGGGCGGTTGGCGATCTGGTGCAG |
| | | GTGGTGAACTCCATCAGCGACGGCGATGTTAACGC |
| | | GCTGGTCGATGAGTACGAAAGCTGCTACACCATGA |
| | | CGCCTGCAACACAAATCCACGGCGAAAAACGACAG |
| | | AACGTGCTGGAAGCGGCGCGTATTGAGCTGGGGAT |
| | | GAAGCGTTTCCTGGAACAAGGTGGCTTCCACGCGT |
| | | TCACCACCACCTTTGAAGATTTGCACGGTCTGAAAC |
| | | AGCTTCCAGGTCTGGCCGTACAGCGTCTGATGCAG |
| | | CAGGGTTACGGCTTTGCGGGCGAAGGCGACTGGAA |
| | | AACCGCCGCCCTGCTTCGCATCATGAAGGTGATGTC |
| | | AACCGGTCTGCAGGGCGGCACCTCCTTTATGGAGG |
| | | ACTACACCTATCACTTCGAGAAAGGTAATGACTTG |
| | | GTGCTCGGCTCCCATATGCTGGAAGTCTGCCCGTCG |
| | | ATTGCCGTAGAAGAGAAACCGATCCTCGACGTTCA |
| | | GCATCTCGGTATTGGTGGTAAGGACGATCCTGCCC |
| | | GACTGATCTTCAATACCCAAACCGGTCCAGCGATT |
| | | GTCGCCAGCCTGATTGATCTCGGCGATCGTTACCGT |
| | | CTGCTGGTTAACTGTATCGACACGGTGAAAACACC |
| | | GCACTCCCTGCCGAAACTGCCGGTGGCGAATGCGC |
| | | TGTGGAAAGCGCAACCGGATCTGCCAACTGCTTCC |
| | | GAAGCGTGGATCCTCGCTGGTGGCGCGCACCATAC |
| | | CGTCTTCAGCCATGCGCTGAACCTCAACGATATGCG |
| | | CCAGTTCGCCGAGATGCACGACATTGAAATCACGG |
| | | TGATTGATAACGATACCCGCCTGCCAGCGTTTAAA |
| | | GACGCGCTGCGCTGGAACGAAGTGTATTACGGATT |
| | | TCGTCGCTAAGTAGTCGCATCAGGTGTGTAACGCCT |
| | | GATGCGGCCTGACGCGTCTTATCAGGCCTACACGCT |
| | | GCGATTTTGTAGGCCGGATAAGCAAAGCGCATCCG |
| | | GCATTCAACGCCTGATGCGACGCTGGCGCGTCTTAT |
| | | CAGGCCTACGCGTTCCGATTTTGTAGGCCGGATAA |
| | | GCAAAGCGCATCCGGCATTCAACGCCTGATGCGAC |
| | | GCTGGCGCGTCTTATCAGGCCTACACGCTGCGATTT |
| | | TGTAGGCCGGATAAGCAAAGCGCATCCGGCACGAA |
| | | GGAGTCAACATGTTAGAAGATCTCAAACGCCAGGT |
| | | ATTAGAGGCCAACCTGGCGCTGCCAAAACATAACC |
| | | TGGTCACGCTCACATGGGGCAACGTCAGCGCCGTT |
| | | GATCGCGAGCGCGGCGTCTTTGTGATCAAACCTTCC |
| | | GGCGTCGATTACAGCATCATGACCGCTGACGATAT |
| | | GGTCGTGGTTAGCATCGAAACCGGTGAAGTGGTTG |
| | | AAGGTGCGAAAAAGCCCTCCTCCGATACGCCAACT |
| | | CACCGACTGCTCTATCAGGCATTCCCGTCCATTGGC |
| | | GGCATTGTGCACACACACTCGCGCCACGCCACTAT |
| | | CTGGGCGCAGGCGGGCCAGTCGATTCCAGCAACCG |
| | | GCACCACCCACGCCGACTATTTCTACGGCACCATTC |
| | | CCTGCACCCGCAAAATGACCGACGCAGAAATCAAC |
| | | GGTGAATATGAGTGGGAAACCGGTAACGTCATCGT |
| | | AGAAACCTTCGAAAAACAGGGTATCGATGCAGCGC |
| | | AAATGCCCGGCGTCCTGGTCCATTCTCACGGCCCAT |
| | | TTGCATGGGGCAAAAATGCCGAAGATGCGGTGCAT |
| | | AACGCCATCGTGCTGGAAGAGGTCGCTTATATGGG |
| | | GATATTCTGCCGTCAGTTAGCGCCGCAGTTACCGGA |
| | | TATGCAGCAAACGCTGCTGAATAAACACTATCTGC |
| | | GTAAGCATGGCGCGAAGGCATATTACGGGCAGTAA |
| | | TGACTGTATAAAACCACAGCCAATCAAACGAAACC |
| | | AGGCTATAATCAAGCCTGGTTTTTTGATGGAATTAC |
| | | AGCGTGGCGCAGGCAGGTTTTATCTTAACCCGACA |
| | | CTGGCGGGACACCCCGCAAGGGACAGAAGTCTCCT |
| | | TCTGGCTGGCGACGGACAACGGGCC |
| | | (SEQ ID NO: 80) |
| Xylulokinase | 2.7.1.17 | >gi\|755795\|emb\|X82408.1\| |
| | | S.cerevisiae XKS1, G7579, G7576 |
| | | and G7572 genes (complement |
| | | 7022-8530) |
| | | GAATTCATAATGTGATAGAATAATGGGTGAAGTGT |
| | | ATAAAGAAGAATATATAATATTACTGTGTAGAAAT |
| | | ATCAATTTCCCTTTGTGAGTTCTCATAACCTCGAGG |
| | | AGAAGTTTTTTTACCCCTCTCCACAGATCGATACTT |
| | | ATCATTAAGAAAATGGGACACCAAGGTTACGGAAA |
| | | AATCTACCCGGTCTACCTAATTACTCTCTTGGCGCA |
| | | CTAGTTTTCCGAAAAAAACAGGTAAATTCTTCTTTA |
| | | GATAAAGATAAATATAAAACTTCACAGCCATTCAC |
| | | TCACACAAACTAGTCCCTTAGGGTGCGTATAATGAT |
| | | CTGTACATCTTATTTCTATATATCTTACCGTGTATTT |
| | | TTTCTTTTCTCAATTCTTGTTCGCAAATAAAAAGAT |
| | | ATTCGTGTTTGTGGAAGAACACTAGTTCCGTTTTGT |
| | | ATTCAACCTGGAAATTTACAATAGATCTTCATCATC |
| | | GTATGTCTACCATGTTAATCTCCCGTTAAACTGTTT |
| | | CACGTTATCAAGATTATGTCATCTATTCCTGGGCGA |
| | | ACATAATTCCTTACAAAAACATTTGTCATTACACAA |
| | | GTGTAAGGGGTAATGAAAAGTAATTTTGTTACAAG |
| | | TACGCAAAATTCGTTTATTTCAAGAAACACTAAGG |
| | | ATCGTCATTTCCCTTTCTGACCGATGTTCCTTCTTTT |
| | | TGCTATTTTTTTCCCGAGTCATCTCATCGTTTTGAGT |
| | | TTTTCCTAGTCCATTAAATTGTCACCTTACTCTCGG |
| | | AAAAAAGAAACGACAAATGCTCCTAGTGCCGTTTT |
| | | TCGAAGCTTGAAAAAAAAAATTGCAAATTATTTAA |
| | | TTTTGCTGCTAAGGAGTTGAAGTAGGTGCATTCCGC |
| | | CTTATTGATCACCCTGTTAGATTTGTTGCGATCGTT |
| | | ATAGTGCTAGTTTGTCCATTGTTGTGTCATAAAAGA |
| | | TAGCTTTGGGAGAAAATTCATCAAAACAACATATC |
| | | ATCAGCGTTATTACAATTCATTGTCCTTCCCAAGTT |
| | | TTTTTGACGTATAATATTATCGCTATCTGACTCATT |
| | | AGTACACAAATACAGATATACAACCTCAAAATCAA |
| | | AAATGCCTAGAAACCCATTGAAAAAGGAATATTGG |
| | | GCAGATGTAGTTGACGGATTCAAGCCGGCTACTTCT |
| | | CCAGCCTTCGAGAATGAAAAAGAATCTACTACATT |
| | | TGTTACCGAACTAACTTCCAAAACCGATTCTGCATT |
| | | TCCATTAAGTAGCAAGGATTCACCTGGCATAAACC |
| | | AAACCACAAACGATATTACCTCTTCAGATCGCTTCC |
| | | GTCGTAATGAAGACACAGAGCAGGAAGACATCAAC |
| | | AACACCAACCTGAGTAAAGATCTATCCGTGAGACA |
| | | TCTTTTAACTCTAGCTGTCGGGGGTGCAATAGGTAC |
| | | TGGTTTATATGTGAATACGGGTGCTGCTTTATCTAC |
| | | AGGTGGTCCGGCCAGTTTAGTTATTGATTGGGTTAT |
| | | TATCAGTACATGTCTTTTTACTGTGATTAACTCTCTT |
| | | GGTGAGCTGTCCGCTGCTTTTCCCGTTGTTGGTGGG |
| | | TTCAATGTTTACAGTATGCGTTTTATTGAGCCTTCA |
| | | TTTGCATTCGCAGTGAACTTAAACTATTTAGCACAA |
| | | TGGCTAGTTCTTCTACCCTTGGAATTAGTGGCCGCA |
| | | TCTATTACTATAAAATACTGGAATGATAAAATTAAT |
| | | TCCGACGCCTGGGTTGCTATCTTTTATGCCACCATT |
| | | GCACTGGCTAATATGTTGGATGTTAAGTCATTTGGT |
| | | GAGACCGAATTTGTATTGTCCATGATTAAAATCCTC |
| | | TCCATCATTGGCTTTACTATCTTAGGTATTGTTTTGT |
| | | CCTGTGGTGGTGGGCCTCACGGCGGTTACATTGGTG |
| | | GTAAATACTGGCATGACCCAGGCGCTTTTGTAGGG |
| | | CACAGCTCGGGAACTCAGTTTAAAGGTTTATGTTCA |
| | | GTTTTTGTTACCGCTGCCTTTTCTTATTCCGGTATTG |
| | | AAATGACTGCTGTCTCCGCTGCTGAAAGTAAAAAT |
| | | CCAAGAGAAACCATTCCCAAGGCAGCAAAGAGAA |
| | | CTTTTTGGCTGATTACCGCCTCTTATGTGACTATATT |
| | | GACTTTGATTGGTTGCTTGGTTCCATCCAATGACCC |
| | | TAGGTTACTAAACGGTTCAAGTTCAGTGGACGCTG |
| | | CCTCATCTCCTCTGGTTATCGCAATTGAAAACGGGG |
| | | GTATTAAAGGTCTACCATCATTAATGAACGCCATTA |
| | | TTTTGATTGCTGTTGTTTCCGTGGCTAACAGTGCTG |
| | | TTTATGCATGTTCAAGGTGTATGGTCGCCATGGCTC |
| | | ATATTGGTAATTTACCAAAATTTTTGAACCGTGTTG |
| | | ACAAAAGGGGTAGACCAATGAATGCTATCTTGTTA |
| | | ACTTTGTTTTTTGGTTTGCTTTCCTTTGTGGCAGCAA |
| | | GTGATAAGCAAGCTGAAGTCTTTACATGGTTGAGT |
| | | GCCTTATCTGGTTTATCGACAATTTTCTGCTGGATG |
| | | GCCATTAATCTTTCCCATATTAGATTTCGCCAAGCC |
| | | ATGAAAGTTCAAGAAAGGTCTTTAGACGAATTACC |
| | | CTTCATTTCTCAAACTGGCGTCAAGGGATCCTGGTA |
| | | TGGTTTTATCGTTTTATTTCTGGTTCTTATAGCATCG |
| | | TTTTGGACTTCTCTGTTCCCATTAGGCGGTTCAGGA |
| | | GCCAGCGCAGAATCATTCTTTGAAGGATACTTATCC |
| | | TTTCCAATTTTGATTGTCTGTTACGTTGGACATAAA |
| | | CTGTATACTAGAAATTGGACTTTGATGGTGAAACTA |
| | | GAAGATATGGATCTTGATACCGGCAGAAAACAAGT |
| | | AGATTTGACTCTTCGTAGGGAAGAAATGAGGATTG |
| | | AGCGAGAAACATTAGCAAAAAGATCCTTCGTAACA |
| | | AGATTTTTACATTTCTGGTGTTGAAGGGAAAGATAT |
| | | GAGCTATACAGCGGAATTTCCATATCACTCAGATTT |
| | | TGTTATCTAATTTTTTCCTTCCCACGTCCGCGGGAA |
| | | TCTGTGTATATTACTGCATCTAGATATATGTTATCTT |
| | | ATCTTGGCGCGTACATTTAATTTTCAACGTATTCTA |
| | | TAAGAAATTGCGGGAGTTTTTTTCATGTAGATGATA |
| | | CTGACTGCACGCAAATATAGGCATGATTTATAGGC |
| | | ATGATTTGATGGCTGTACCGATAGGAACGCTAAGA |
| | | GTAACTTCAGAATCGTTATCCTGGCGGAAAAAATT |
| | | CATTTGTAAACTTTAAAAAAAAAAGCCAATATCCC |
| | | CAAAATTATTAAGAGCGCCTCCATTATTAACTAAA |
| | | ATTTCACTCAGCATCCACAATGTATCAGGTATCTAC |
| | | TACAGATATTACATGTGGCGAAAAAGACAAGAACA |
| | | ATGCAATAGCGCATCAAGAAAAAACACAAAGCTTT |
| | | CAATCAATGAATCGAAAATGTCATTAAAATAGTAT |
| | | ATAAATTGAAACTAAGTCATAAAGCTATAAAAAGA |
| | | AAATTTATTTAAATGCAAGATTTAAAGTAAATTCAC |
| | | TTAAGCCTTGGCAACGTGTTCAACCAAGTCGACAA |
| | | CTCTGGTAGAGTAACCGTATTCGTTGTCGTACCAGG |
| | | AGACCAACTTGACGAACTTTGGAGACAATTGGATA |
| | | CCAGCGGAAGCATCGAAGATGGAAGAGTGAGAGT |
| | | CACCCAAGAAGTCAGAGGAGACAACAGCGTCTTCG |
| | | GTGTAACCCAAAACACCCTTCAACTTACCTTCAGCG |
| | | GCAGCCTTAACAACCTTCTTGATTTCATCGTAGGTG |
| | | GTTTCCTTGTTCAACTTGACAGTCAAGTCAACAACG |
| | | GAGACATCGACGGTTGGGACTCTGAAAGCCATACC |
| | | GGTCAACTTACCTTGCAATTCTGGCAAGACCTTACC |
| | | GACAGCCTTAGCAGCACCGGTGGAGGATGGGATGA |
| | | TGTTACCGGAAGCGGTTCTACCACCTCTCCAGTCCT |
| | | TGTGGGATGGACCGTCAACAGTCTTTTGAGTAGCA |
| | | GTCAAAGAGTGGACAGTGGTCATCAAACCTTCTTC |
| | | AATACCGAAAGCATCGTTGATAACCTTGGCCAATG |
| | | GAGCCAAACAGTTGGTGGTACAAGAAGCGTTGGAA |
| | | ACAATCTTCAAGTCAGAAGTGTATTTTTCTTCGTTA |
| | | ACACCCATGACGAACATTGGGGCGGTGGAAGATGG |
| | | AGCAGTGATAACAACCTTCTTGGCACCAGCGTCAA |
| | | TGTGCTTTTGAGCAGTGTCTAATTCCTTGAAAACAC |
| | | CAGTGGAGTCAATGGCGATGTCAACGTTGGAAGAA |
| | | CCCCATGGCAAGTTAGCTGGGTCTCTTTCTTGGTAA |
| | | GTAGCAATCTTCTTACCATCGACAATGATGTGCTTG |
| | | TCATCGTGGGAAACTTCACCAGCGTATCTACCGTGA |
| | | GTGGAGTCGTACTTGAACATGTAAGCAGCGTAGTC |
| | | GTTGGTGATGAATGGGTCGTTCAAAGCAACAACTT |
| | | CGACGTTTGGTCTAGACAAAGCAATTCTCATGACC |
| | | AATCTACCGATTCTACCGAAACCGTTAATAGCAACT |
| | | CTAACCATTTTGTTTGTTTATGTGTGTTTATTCGAAA |
| | | CTAAGTTCTTGGTGTTTTAAAACTAAAAAAAAGACT |
| | | AACTATAAAAGTAGAATTTAAGAAGTTTAAGAAAT |
| | | AGATTTACAGAATTACAATCAATACCTACCGTCTTT |
| | | ATATACTTATTAGTCAAGTAGGGGAATAATTTCAG |
| | | GGAACTGGTTTCAACCTTTTTTTTCAGCTTTTTCCAA |
| | | ATCAGAGAGAGCAGAAGGTAATAGAAGGTGTAAG |
| | | AAAATGAGATAGATACATGCGTGGGTCAATTGCCT |
| | | TGTGTCATCATTTACTCCAGGCAGGTTGCATCACTC |
| | | CATTGAGGTTGTGCCCGTTTTTTGCCTGTTTGTGCCC |
| | | CTGTTCTCTGTAGTTGCGCTAAGAGAATGGACCTAT |
| | | GAACTGATGGTTGGTGAAGAAAACAATATTTTGGT |
| | | GCTGGGATTCTTTTTTTTTCTGGATGCCAGCTTAAA |
| | | AAGCGGGCTCCATTATATTTAGTGGATGCCAGGAA |
| | | TAAACTGTTCACCCAGACACCTACGATGTTATATAT |
| | | TCTGTGTAACCCGCCCCCTATTTTGGGCATGTACGG |
| | | GTTACAGCAGAATTAAAAGGCTAATTTTTTGACTAA |
| | | ATAAAGTTAGGAAAATCACTACTATTAATTATTTAC |
| | | GTATTCTTTGAAATGGCAGTATTGATAATGATAAAC |
| | | TCGAACTGAAAAAGCGTGTTTTTTATTCAAAATGAT |
| | | TCTAACTCCCTTACGTAATCAAGGAATCTTTTTGCC |
| | | TTGGCCTCCGCGTCATTAAACTTCTTGTTGTTGACG |
| | | CTAACATTCAACGCTAGTATATATTCGTTTTTTTCA |
| | | GGTAAGTTCTTTTCAACGGGTCTTACTGATGAGGCA |
| | | GTCGCGTCTGAACCTGTTAAGAGGTCAAATATGTCT |
| | | TCTTGACCGTACGTGTCTTGCATGTTATTAGCTTTG |
| | | GGAATTTGCATCAAGTCATAGGAAAATTTAAATCTT |
| | | GGCTCTCTTGGGCTCAAGGTGACAAGGTCCTCGAA |
| | | AATAGGGTCAAAGTATTTCGAATTTGTGTCCATTAG |
| | | TGGTGTTCCGTGTGAGAACTGGTAAGCTTCTTTCAT |
| | | GAAAGAGTTCAATGATTCACTCAGTTTGTCAAACG |
| | | GAATAGAGGCGGGAGCATGGAACACTAATTGCTCT |
| | | TCAAATTCTACAGGAGTAATCTTTGGCTTGTCGGCA |
| | | GCTTTTGTTTGAGCTTGCTCGGCTATTTTTGGTTTGA |
| | | GCTGTATAGGTTTAATGTTCGATAAATCGAGACGTT |
| | | CGTTCTTCTTGATAAATTCTGTTACCTTGTTAACCG |
| | | AATCTTGTGGTATTTTCCCTAGGTATGCTAGCACAT |
| | | CACCCTTTAATAGTCTACCGTTGGAACCAGATGGCG |
| | | CAATTTCCTTCAAAGCCTTTTGTTTGGATATATTGTT |
| | | CTCAGCCAGTAGTAATGACACGGATGGTAATAGCG |
| | | TCTGTTCAAGATTGGCTTGGCTGCCGTCAACGGTTT |
| | | TTATTGGTGTAACTGTGGCTTTTTTTAAATGTTGTTG |
| | | TGTTGCTTCAGTACTATCTGCGGATGGCTTCTTAAT |
| | | TTCAATAGATTTCGCATTTGCGGTGTTGGCCTCTTG |
| | | GGGTAACTTTATAGTAGCTAAATCATCATCAACATC |
| | | AGCAATATAAGCAATAGGTTCACCAACATCAACAT |
| | | CTTTAGAGCCTTCATCTTTCAGGATCTTAGCTAGTT |
| | | TACCATCGTCCAGTGCTTCCACATCAATTTGAGATT |
| | | TATCTGTTTCCACTTCTAATATCACATCGCCCGCGC |
| | | TGAATGGTTCGCCAACTTTATATTTCCAAGACACAA |
| | | TCCCCCCTTTCTCCATAGTAGGAGACATTGCAGGCA |
| | | TTGAAAATGTCTTTACAGCAAGTAATTTAGCTGATG |
| | | CATGATAGTTGCATTTGGTTAAATATCTTGTACATG |
| | | ATTTTAAAGTGGAGACTTTGGAAATTGCACTTAGCA |
| | | TTTTTGAATTTTTCCCTCGAGATGATTTAACAATAA |
| | | CCTAGCTCTTTCAATGCTCTCTTATATTCTCACTGGA |
| | | AAGCGCTAATTTGATTTGTCTCTCTCGTTGCTGGTC |
| | | GCTCACCCTTTCATAAATTGTTTTTTACTCTTCATTT |
| | | ATTGATTTTACTTTTTGTCATTTTCCGAACGGGGAA |
| | | CAAAATGATGACTACTTGCTACAGTATACGAAACA |
| | | TACAAGGGCATTGTCATGTGCACGCATAATAACGG |
| | | TGATATATATATATGTATGTATATTATGTGTCTGTG |
| | | TTTGTGTGTACTTGTCAGGGCATGATAAATTATTCA |
| | | AACATATTTTAGATGAGAGTCTTTTCCAGTTCGCTT |
| | | AAGGGGACAATCTTGGAATTATAGCGATCCCAATT |
| | | TTCATTATCCACATCGGATATGCTTTCCATTACATG |
| | | CCATGGAAAATTGTCATTCAGAAATTTATCAAAAG |
| | | GAACTGCAATTTTATTAGAGTCATATAACAATGACC |
| | | ACATGGCCTTATAACAACCACCAAGGGCACATGAG |
| | | TTTGGTGTTTCTAGCCTAAAATTACCCTTTGTAGCA |
| | | CCAATGACTTGAGCAAACTTCTTCACAATAGCATCG |
| | | TTTTTAGAAGCCCCACCTACAAAAAAAGTCCTTTCT |
| | | GCCTTTTATTTAGGTAGTCCCGCAGCGGAGATTCAT |
| | | CGTAATCAAACTTCACGATTGTATCTTCGTTCAGTC |
| | | TCTGTTGTGAGCTTGCGTTTGAATCCGAAAGCAGGG |
| | | GAGATATTCTTACCCTGCAACTTAAAGCCTGTGATT |
| | | CTACAATATTTTTGGCATCGTGCCTCTTGTCTTTGA |
| | | ACTTGGCCACCTCTCTTTCAATCATACCCGTTTTTG |
| | | GATTGAAGATAACCCTTTTGTTTATGGCTTTTACGC |
| | | TAGGAACGATCTCCCCCAGAGGAAAATATACACCT |
| | | AATTCATTTTCACTACTTTCTGAGTCATCTAGCACA |
| | | GCTTGATTAAAAAGAGTCCAATCGTTAGTCTTCTCA |
| | | TAATTATTTTCCCGTTCTTTGTTTAACTCGTCTCTTA |
| | | TCCTCTCCCTTGCCAAAGAACCATTACAATAACAAA |
| | | TCATACCCATATAATGGTTTGGCAGAGTTGGATGA |
| | | ATGAAAAGATGATAGTTCGGAGAGGGGTGATACTT |
| | | ATCGGTGACCAGAAGAACTGTAGTACTTGTTCCTA |
| | | GGGAAACGAGAACGTCATTCTTCCGCAGGGGTAAA |
| | | GAACATATAGTGGCTAAATTATCCCCAGTCATGGG |
| | | AGAGACCTTGCAGTTTGTATTGAAACCGTACTTCTC |
| | | AATAAAATATTTACAGATGGTACCCGCTATCAAATT |
| | | TTTCATGGGTGCTCTCATTAATTTTTGTCTGATAGTT |
| | | TTATCCTTAGAAGAACTATCAATTAGATGTAGTAGC |
| | | TCATCACTGAATTTTCTTTCACGTATATCATAAAGG |
| | | TTCATACCACAGGCATCTGCCTCCTCTAATTCAACA |
| | | AGATGGCCCACTAAGATAGAAGTCAAAAAATTAGA |
| | | CACTAAAGAAATGGTCTTTGTTTTTTCGTAAGCTTC |
| | | TGGTTCTAATTGTGCAATTTTCAGAATTTGAGGACC |
| | | AGTAAATCTAAAATGGGCTCTGGACCCTGTTAATTG |
| | | AGCCATTTTTTCAGGCCCACCTATGCACTCTTCAAA |
| | | CTCTTGACATTGCTTTGCAGTACTGTGGTCTTGCCA |
| | | ATTGGGGGCGGTTTGCCTTGCAAATGCTACAGAGC |
| | | TCACGTAGTGCAATAAATCTTTTTCCGGTTTCTTATT |
| | | CAATTGCTCTAACAGAGATTCGGCTTGGGAGGACC |
| | | AGTAGACAGACCCGTGCTGCTGGCAGGACCCTGAG |
| | | ACGGCCATAACTTTGTTCAATGGAAATTTAGCCTCG |
| | | CGATATTTCGAGAGAACCAGATCTAGAGCCTCTAA |
| | | CCACATGGCTACGGGACATTCGATAGTGTCGCCGT |
| | | GTATATAGACACCCTTCTTTGTGTGATAATGCGGAA |
| | | GATCCTTTTCAAATTCCACTGTTTCTGAATGGACAA |
| | | TTTTTAGGTCCTGGTTAATGGCGAGACATTTCAGTT |
| | | GTTGGGTCGAAAGATCAAACCCAAGATAGTATGAG |
| | | TCTAAAGACATTGTGTTGGAAACCTCTCTTGTCTGT |
| | | CTCTGAATTACTGAACACAACATTAAAGTACTAATC |
| | | TCATCCTCCTTTTGTTTTTCTCGAGAGGGCCCCCTTA |
| | | TTCGTCCGCCTGAGATTGCATTGGCCGAATTGAAAA |
| | | GTGACAGTTATGCACCTCAGCGGCTATTCCCTGCGT |
| | | CGCTTGCTGAAGATTGAGGATTATAGGAAGTAGTA |
| | | AGCTGGAAATGGTAATTTGTAAAAAGAAAATTTCT |
| | | AATACTGTGACAATGTTATTAAATCGGGGTTGTTTT |
| | | TGTTTGGGGCGGCCAGCGGAATAATTTGTTTTCAAG |
| | | GACAGCAGAAGCTCAGAAGAACAAAATCTCCGTGA |
| | | TCTTTTAAACTTTTTTCCATTCTGATGGAATAATGGT |
| | | CTTGATCCCCTAAATATTTCCTTTTTTATTGCAATTA |
| | | TGATAGAAATTAATAGTAGTCTATGCTGGATGATAT |
| | | ATATATCTCTTTTGAGCTTGCTTCAAATTTTTTCCCG |
| | | TTATAATGATGAGTGTTTTGCTTATTAAGGGTCTAG |
| | | GACATTTCTATCAGTTTCTATACCA |
| | | (SEQ ID NO: 81) |
| L-Ribulose-5- | 5.1.3.4 | >gi\|40938\|emb\|X56048.1\| E. coli |
| phosphate 4- | | araD gene for L-ribulose-phosphate |
| epimerase | | 4-epimerase (EC 5.1.3.4)(466-1161) |
| | | GGATCCTCGCTGGTGGCGCGCACCATACCGTCTTCA |
| | | GCCATGCACTGAACCTCAACGATATGCGCCAATTC |
| | | GCCGGAGATGCACGACATTGAAATCACGGTGATTG |
| | | ATAACGACACCCGCCTGCCAGCGTTTAAAGACGCG |
| | | CTGCGCTGGAACGGAAGTGTATTACGGGTTTCGTC |
| | | GCTAAGTAGCCGCATCCGGTATGTAACGCCTGATG |
| | | CGACGCTGACGCGTCTTATCTGGCCTACACGCTGCG |
| | | ATTTTGTAGGCCGGATAAGCAAAGCGCATCCGGCA |
| | | TTCAACGCCTGATGCGACGCTGGCGCGTCTTATCAG |
| | | GCCTACGCGCTGCGATTTTGTAGGCCGGATAAGCA |
| | | AAGCGCATCCGGCATTCAACGCCTGATGCGACGCT |
| | | GGCGCGTCTTATCAGGCCTACACGCTGCGATTTTGT |
| | | AGGCCGGATAAGCAAAGCGCATCCGGCACGAAGG |
| | | AGTCAACATGTTAGAAGATCTCAAACGCCAGGTAT |
| | | TAGAAGCCAACCTGGCGCTGCCAAAACACAACCTG |
| | | GTCACGCTCACATGGGGCAACGTCAGCGCCGTTGA |
| | | TCGCGAGCGCGGCGTCTTTGTGATCAAACCTTCCGG |
| | | CGTCGATTACAGCGTCATGACCGCTGACGATATGG |
| | | TCGTGGTTAGCATCGAAACCGGTGAAGTGGTTGAA |
| | | GGTACGAAAAAGCCCTCCTCCGACACGCCAACTCA |
| | | CCGGCTGCTCTATCAGGCATTCCCCTCCATTGGCGG |
| | | CATTGTGCATACGCACTCGCGCCACGCCACCATCTG |
| | | GGCGCAGGCGGGTCAGTCGATTCCAGCAACCGGCA |
| | | CCACCCACGCCGACTATTTCTACGGCACCATTCCCT |
| | | GCACCCGCAAAATGACCGACGCAGAAATCAACGGC |
| | | GAATATGAGTGGGAAACCGGTAACGTCATCGTAGA |
| | | AACCTTTGAAAAACAGGGTATCGATGCAGCGCAAA |
| | | TGCCCGGCGTTCTGGTCCATTCCCACGGCCCGTTTG |
| | | CATGGGGCAAAAATGCCGAAGATGCGGTGCATAAC |
| | | GCCATCGTGCTGGAAGAGGTCGCTTATATGGGGAT |
| | | ATTCTGCCGTCAGTTAGCGCCGCAGTTACCGGATAT |
| | | GCAGCAAACGCTGCTGGATAAACACTATCTGCGTA |
| | | AGCATGGCGCGAAGGCATATTACGGGCAGTAATGA |
| | | CTGTATAAAACCACAGCCAATCAAACGAAACCAGG |
| | | CTATACTCAAGCCTGGTT |
| | | (SEQ ID NO: 82) |
| L-Arabinose | 5.3.1.4 | >gi\|1924929\|emb\|X89408.1\| |
| isomerase | | B.subtilis DNA for araA, araB |
| (araA) | | and araD genes (228-1718) |
| | | AAGCTTCTCATCAATGATTTGAATTGGAGCTCGGGC |
| | | TGGCCGTCCTATTGAATTAAAAAGCCGGCTCTGCCC |
| | | CCGGCTTTTTTTAAAAGAAAAGATTGACAGTATAAT |
| | | AGTCAATTACTATAATAAAATTGTTCGTACAAATAT |
| | | TTATTTATAGGTTTTATTTTCTAATTAGTACGTATCT |
| | | TTTGTATTTGAAAGCGTTTTATTTTATGAGAAAGGG |
| | | GCAGTTTACATGCTTCAGACAAAGGATTATGAATTC |
| | | TGGTTTGTGACAGGAAGCCAGCACCTATACGGGGA |
| | | AGAGACGCTGGAACTCGTAGATCAGCATGCTAAAA |
| | | GCATTTGTGAGGGGCTCAGCGGGATTTCTTCCAGAT |
| | | ATAAAATCACTCATAAGCCCGTCGTCACTTCACCGG |
| | | AAACCATTAGAGAGCTGTTAAGAGAAGCGGAGTAC |
| | | AGTGAGACATGTGCTGGCATCATTACATGGATGCA |
| | | CACATTTTCCCCTGCAAAAATGTGGATAGAAGGCC |
| | | TTTCCTCTTATCAAAAACCGCTTATGCATTTGCATA |
| | | CCCAATATAATCGCGATATCCCGTGGGGTACGATT |
| | | GACATGGATTTTATGAACAGCAACCAATCCGCGCA |
| | | TGGCGATCGAGAGTACGGTTACATCAACTCGAGAA |
| | | TGGGGCTTAGCCGAAAAGTCATTGCCGGCTATTGG |
| | | GATGATGAAGAAGTGAAAAAAGAAATGTCCCAGTG |
| | | GATGGATACGGCGGCTGCATTAAATGAAAGCAGAC |
| | | ATATTAAGGTTGCCAGATTTGGAGATAACATGCGT |
| | | CATGTCGCGGTAACGGACGGAGACAAGGTGGGAGC |
| | | GCATATTCAATTTGGCTGGCAGGTTGACGGATATG |
| | | GCATCGGGGATCTCGTTGAAGTGATGGATCGCATT |
| | | ACGGACGACGAGGTTGACACGCTTTATGCCGAGTA |
| | | TGACAGACTATATGTGATCAGTGAGGAAACAAAAC |
| | | GTGACGAAGCAAAGGTAGCGTCCATTAAAGAACAG |
| | | GCGAAAATTGAACTTGGATTAACCGCTTTTCTTGAG |
| | | CAAGGCGGATACACAGCGTTTACGACATCGTTTGA |
| | | AGTGCTGCACGGAATGAAACAGCTGCCGGGACTTG |
| | | CCGTTCAGCGCCTGATGGAGAAAGGCTATGGGTTT |
| | | GCCGGTGAAGGAGATTGGAAGACAGCGGCCCTTGT |
| | | ACGGATGATGAAAATCATGGCTAAAGGAAAAAGA |
| | | ACTTCCTTCATGGAAGATTACACGTACCATTTTGAA |
| | | CCGGGAAATGAAATGATTCTGGGCTCTCACATGCTT |
| | | GAAGTGTGTCCGACTGTCGCTTTGGATCAGCCGAA |
| | | AATCGAGGTTCATTCGCTTTCGATTGGCGGCAAAG |
| | | AGGACCCTGCGCGTTTGGTATTTAACGGCATCAGC |
| | | GGTTCTGCCATTCAAGCTAGCATTGTTGATATTGGC |
| | | GGGCGTTTCCGCCTTGTGCTGAATGAAGTCAACGG |
| | | CCAGGAAATTGAAAAAGACATGCCGAATTTACCGG |
| | | TTGCCCGTGTTCTCTGGAAGCCGGAGCCGTCATTGA |
| | | AAACAGCAGCGGAGGCATGGATTTTAGCCGGCGGT |
| | | GCACACCATACCTGCCTGTCTTATGAACTGACAGCG |
| | | GAGCAAATGCTTGATTGGGCGGAAATGGCGGGAAT |
| | | CGAAAGTGTTCTCATTTCCCGTGATACGACAATTCA |
| | | TAAACTGAAACACGAGTTAAAATGGAACGAGGCGC |
| | | TTTACCGGCTTCAAAAGTAGAGGGGGATGTCACAT |
| | | GGCTTACACAATAGGGGTTGATTTTGGAACTTTATC |
| | | AGGAAGAGCAGTGCTCGTTCATGTCCAAACAGGGG |
| | | AGGAACTTGCGGCTGCTGTAAAAGAATACAGGCAT |
| | | GCTGTCATTGATACCGTCCTTCCAAAAACGGGTCAA |
| | | AAGCTGCCGCGTGACTGGGCGCTGCAGCATTTTGCT |
| | | GATTACCTCGAAGTCTTGGAAACAACCATTCCGTCT |
| | | TTACTCGAACAGACGGGCGTTGACCCGAAAGACAT |
| | | TATCGGGATTGGAATTGATTTCACGGCATGTACGAT |
| | | CCTTCCTATTGACAGCAGCGGGCAGCCGTTATGCAT |
| | | GCTGCCTGAATATGAAGAGGAGCCGCACAGCTATG |
| | | TGAAGCTCTGGAAGCATCATGCGGCCCAAAAACAT |
| | | GCTGATCGGCTCAATCAAATCGCGGAAGAAGAAGG |
| | | AGAGGCTTTTTTACAGCGGTACGGAGGAAAAATTT |
| | | CATCAGAATGGATGATTCCAAAGGTCATGCAAATT |
| | | GCCGAGGAAGCGCCTCACATTTATGAAGCGGCTGA |
| | | CCGGATCATCGAGGCTGCGGACTGGATCGTGTACC |
| | | AGCTGTGCGGCTCGCTCAAGCGAAGCAATTGCACC |
| | | GCAGGGTATAAAGCGATGTGGAGTGAAAAAGCGG |
| | | GGTATCCGTCAGATGATTTCTTTGAGAAATTAAATC |
| | | CTTCAATGAAAACGATTACAAAGGACAAATTGTCA |
| | | GGTTCTATTCATTCAGTAGGAGAAAAAGCCGGCAG |
| | | TCTGACTGAAAAAATGGCAAAGCTGACAGGGCTTC |
| | | TCCCGGGAACGGCTGTTGCGGTTGCCAATGTGGAC |
| | | GCTCATGTTTCGGTACCGGCGGTCGGCATTACAGA |
| | | GCCAGGGAAAATGCTGATGATTATGGGAACCTCGA |
| | | CGTGCCATGTTCTACTTGGTGAAGAGGTGCATATCG |
| | | TTCCAGGAATGTGCGGCGTTGTGGACAACGGAATT |
| | | CTCCCGGGCTATGCGGGATATGAAGCCGGGCAGTC |
| | | CTGTGTCGGCGATCATTTTGACTGGTTTGTGAAAAC |
| | | ATGTGTCCCGCCAGCTTATCAAGAGGAAGCAAAGG |
| | | AAAAAAACATTGGCGTTCATGAGCTGCTGAGTGAG |
| | | AAAGCAAACCATCAAGCGCCTGGTGAAAGCGGCTT |
| | | GCTTGCTTTAGATTGGTGGAATGGAAACCGTTCAAC |
| | | TCTTGTTGATGCAGATTTAACAGGGATGCTGCTTGG |
| | | CATGACACTGCTGACGAAGCCTGAAGAGATTTATA |
| | | GAGCGTTAGTTGAAGCGACAGCTTACGGAACCCGG |
| | | ATGATTATCGAAACATTCAAAGAAAGCGGTGTTCC |
| | | GATTGAGGAACTGTTCGCAGCCGGCGGAATAGCTG |
| | | AGAAAAACCCGTTTGTCATGCAGATTTATGCGGAT |
| | | GTGACAAACATGGACATTAAAATCTCTGGTTCACC |
| | | GCAAGCCCCAGCCTTAGGATCTGCCATTTTCGGCGC |
| | | GCTTGCAGCAGGCAAAGAAAAAGGCGGCTACGATG |
| | | ATATCAAAAAGGCAGCGGCGAACATGGGAAAACT |
| | | GAAAGATATAACTTATACGCCAAATGCCGAAAACG |
| | | CCGCGGTTTATGAAAAATTGTACGCTGAATATAAA |
| | | GAGCTGGTTCATTATTTCGGAAAAGAAAACCATGT |
| | | CATGAAGCGTCTGAAAACGATCAAAAATCTTCAAT |
| | | TTTCATCTGCCGCCAAAAAGAATTGATAAAGGGTG |
| | | ATGGAGCATGCTTGAAACATTAAAAAAAGAAGTGC |
| | | TGGCTGCCAACCTGAAGCTTCAAGAGCATCAGCTG |
| | | GTAACCTTTACGTGGGGAAATGTCAGCGGCATTCA |
| | | CCGTGAAAAAGAAAGAATTGTCATCAAACTAGCGG |
| | | AGTCGAATACCAGCGACCTGACAGCCGATGACTTG |
| | | GTTGTTTTGAACCTTGATGGAGAGGTCGTCGAAGG |
| | | CTCGCTTAAACCTTCTTCAGATACACCTACCCATGT |
| | | TTATCTATATAAAGCCTTTCCGAATATCGGGGGAAT |
| | | TGTCCATACCCATTCTCAATGGGCGACAAGCTGGG |
| | | CGCAATCGGGCAGAGACATCCCTCCGTTAGGCACG |
| | | ACCCATGCTGATTATTTTGACAGTGCGATTCCATGT |
| | | ACTCGAGAAATGTACGATGAAGAAATCATTCATGA |
| | | CTACGAACTGAATACAGGAAAAGTCATAGCGGAAA |
| | | CCTTTCAGCATCATAATTACGAACAGGTGCCGGGT |
| | | GTGCTCGTGAATAATCACGGACCGTTCTGCTGGGG |
| | | CACTGACGCCTTAAATGCCATTCATAACGCAGTTGT |
| | | ATTAGAAACGCTTGCCGAAATGGCCTATCACTCCAT |
| | | TATGCTGAACAAGGATGTAACCCCAATCAATACAG |
| | | TCCTGCATGAAAAGCATTTTTATCGAAAACACGGA |
| | | GCAAATGCGTATTATGGCCAGTCATGATACGCCTGT |
| | | GTCACCGGCTGGCATTCTGATTGACTTGGACGGTAC |
| | | TGTATTCAGAGGAAATGAGTTGATCGAAGGAGCAA |
| | | (SEQ ID NO: 83) |
| Xylose | 5.3.1.5 | >gi\|7161892\|emb\|AJ249909.1\| |
| isomerase | | Piromyces sp. E2 mRNA for xylose |
| | | isomerase (xylA gene)(5-1318) |
| | | GTAAATGGCTAAGGAATATTTCCCACAAATTCAAA |
| | | AGATTAAGTTCGAAGGTAAGGATTCTAAGAATCCA |
| | | TTAGCCTTCCACTACTACGATGCTGAAAAGGAAGT |
| | | CATGGGTAAGAAAATGAAGGATTGGTTACGTTTCG |
| | | CCATGGCCTGGTGGCACACTCTTTGCGCCGAAGGT |
| | | GCTGACCAATTCGGTGGAGGTACAAAGTCTTTCCC |
| | | ATGGAACGAAGGTACTGATGCTATTGAAATTGCCA |
| | | AGCAAAAGGTTGATGCTGGTTTCGAAATCATGCAA |
| | | AAGCTTGGTATTCCATACTACTGTTTCCACGATGTT |
| | | GATCTTGTTTCCGAAGGTAACTCTATTGAAGAATAC |
| | | GAATCCAACCTTAAGGCTGTCGTTGCTTACCTCAAG |
| | | GAAAAGCAAAAGGAAACCGGTATTAAGCTTCTCTG |
| | | GAGTACTGCTAACGTCTTCGGTCACAAGCGTTACAT |
| | | GAACGGTGCCTCCACTAACCCAGACTTTGATGTTGT |
| | | CGCCCGTGCTATTGTTCAAATTAAGAACGCCATAG |
| | | ACGCCGGTATTGAACTTGGTGCTGAAAACTACGTCT |
| | | TCTGGGGTGGTCGTGAAGGTTACATGAGTCTCCTTA |
| | | ACACTGACCAAAAGCGTGAAAAGGAACACATGGCC |
| | | ACTATGCTTACCATGGCTCGTGACTACGCTCGTTCC |
| | | AAGGGATTCAAGGGTACTTTCCTCATTGAACCAAA |
| | | GCCAATGGAACCAACCAAGCACCAATACGATGTTG |
| | | ACACTGAAACCGCTATTGGTTTCCTTAAGGCCCACA |
| | | ACTTAGACAAGGACTTCAAGGTCAACATTGAAGTT |
| | | AACCACGCTACTCTTGCTGGTCACACTTTCGAACAC |
| | | GAACTTGCCTGTGCTGTTGATGCTGGTATGCTCGGT |
| | | TCCATTGATGCTAACCGTGGTGACTACCAAAACGG |
| | | TTGGGATACTGATCAATTCCCAATTGATCAATACGA |
| | | ACTCGTCCAAGCTTGGATGGAAATCATCCGTGGTG |
| | | GTGGTTTCGTTACTGGTGGTACCAACTTCGATGCCA |
| | | AGACTCGTCGTAACTCTACTGACCTCGAAGACATC |
| | | ATCATTGCCCACGTTTCTGGTATGGATGCTATGGCT |
| | | CGTGCTCTTGAAAACGCTGCCAAGCTCCTCCAAGA |
| | | ATCTCCATACACCAAGATGAAGAAGGAACGTTACG |
| | | CTTCCTTCGACAGTGGTATTGGTAAGGACTTTGAAG |
| | | ATGGTAAGCTCACCCTCGAACAAGTTTACGAATAC |
| | | GGTAAGAAGAACGGTGAACCAAAGCAAACTTCTGG |
| | | TAAGCAAGAACTCTACGAAGCTATTGTTGCCATGT |
| | | ACCAATAAGTTAATCGTAGTTAAATTGGTAAAATA |
| | | ATTGTAAAATCAATAAACTTGTCAATCCTCCAATCA |
| | | AGTTTAAAAGATCCTATCTCTGTACTAATTAAATAT |
| | | AGTACAAAAAAAAATGTATAAACAAAAAAAAGTCT |
| | | AAAAGACGGAAGAATTTAATTTAGGGAAAAAATAA |
| | | AAATAATAATAAACAATAGATAAATCCTTTATATT |
| | | AGGAAAATGTCCCATTGTATTATTTTCATTTCTACT |
| | | AAAAAAGAAAGTAAATAAAACACAAGAGGAAATT |
| | | TTCCCTTTTTTTTTTTTTTGTAATAAATTTTATGCAA |
| | | ATATAAATATAAATAAAATAATAAAAAAAAAAAA |
| | | AAAAAA |
| | | (SEQ ID NO: 84) |

TABLE 4
SEQ ID numbers of Coding Regions and Proteins for xylose isomerases.
Uniprot accession number (AC) or NCBI GI number given.

| | | SEQ ID NO: | SEQ ID NO: |
|---|---|---|---|
| | | Nucleic | Amino |
| Organism | GI or AC# | Acid | Acid |
| Clavibacter | B0RIF1 | 89 | 90 |
| michiganensis | | | |
| Arthrobacter | 220912923 | 91 | 92 |
| chlorophenolicus | | | |
| Actinosynnema mirum | 226865307 | 93 | 94 |
| Kribbella flavida | 227382478 | 95 | 96 |
| Mycobacterium | 118469437 | 97 | 98 |
| smegmatis | | | |
| Arthrobacter sp. | 60615686 | 99 | 100 |
| Actinomyces | 227497116 | 101 | 102 |
| urogenitalis | | | |
| Streptomyces | 126348424 | 103 | 104 |
| ambofaciens | | | |
| Salinispora arenicola | 159039501 | 105 | 106 |
| Streptomyces sp. | 38141596 | 107 | 108 |
| Meiothermus silvanus | 227989553 | 109 | 110 |
| Actinoplanes sp. | P10654 | 111 | 112 |
| Mobiluncus curtisii | 227493823 | 113 | 114 |
| Herpetosiphon | 159898286 | 115 | 116 |
| aurantiacus | | | |
| Acidothermus | 117929271 | 117 | 118 |
| cellulolyticus | | | |
| Streptomyces coelicolor | Q9L0B8 | 119 | 120 |
| Streptomyces avermitilis | Q93HF3 | 121 | 122 |
| Nocardiopsis | 229207664 | 123 | 124 |
| dassonvillei | | | |
| Nakamurella multipartita | 229221673 | 125 | 126 |
| Xylanimonas | 227427650 | 127 | 128 |
| cellulosilytica | | | |
| Clavibacter | A5CPC1 | 129 | 130 |
| michiganensis | | | |
| Salinispora tropica | 145596104 | 131 | 132 |
| Streptomyces sp. | 197764953 | 133 | 134 |
| Streptomyces | 197776540 | 135 | 136 |
| pristinaespiralis | | | |
| Roseiflexus sp. | 148656997 | 137 | 138 |
| Meiothermus ruber | 227992647 | 139 | 140 |
| Arthrobacter sp. | P12070 | 141 | 142 |
| Thermobaculum | 227374836 | 143 | 144 |
| terrenum | | | |
| Janibacter sp. | 84495191 | 145 | 146 |
| Brachybacterium | 237671435 | 147 | 148 |
| faecium | | | |
| Beutenbergia cavernae | 229821786 | 149 | 150 |
| Geodermatophilus | 227404617 | 151 | 152 |
| obscurus | | | |
| Actinoplanes | P12851 | 153 | 154 |
| missouriensis | | | |
| Streptomyces | P09033 | 155 | 156 |
| violaceusniger | | | |
| Actinomyces | 154508186 | 157 | 158 |
| odontolyticus | | | |
| Mobiluncus mulieris | 227875705 | 159 | 160 |
| Cellulomonas flavigena | 229243977 | 161 | 162 |
| Saccharomonospora | 229886404 | 163 | 164 |
| viridis | | | |
| Streptomyces lividans | Q9RFM4 | 165 | 166 |
| Frankia sp. | 158316430 | 167 | 168 |
| Streptosporangium | 229851079 | 169 | 170 |
| roseum | | | |
| Nocardioides sp. | 119716602 | 171 | 172 |
| Kribbella flavida | 227381155 | 173 | 174 |
| Roseiflexus castenholzii | 156742580 | 175 | 176 |
| Arthrobacter aurescens | 119964059 | 177 | 178 |
| Leifsonia xyli | 50954171 | 179 | 180 |
| Jonesia denitrificans | 227383768 | 181 | 182 |
| Streptomyces | Q93RJ9 | 183 | 184 |
| olivaceoviridis | | | |
| Stackebrandtia | 229862570 | 185 | 186 |
| nassauensis | | | |
| Thermus thermophilus | P26997 | 187 | 188 |
| Acidobacteria bacterium | 94967932 | 189 | 190 |
| Catenulispora acidiphila | 229246901 | 191 | 192 |
| Streptomyces | Q9S3Z4 | 193 | 194 |
| corchorusii | | | |
| Streptomyces | Q9L558 | 195 | 196 |
| thermocyaneoviolaceus | | | |
| marine actinobacterium | 88856315 | 197 | 198 |
| Micromonospora sp. | 237882534 | 199 | 200 |
| Thermobifida fusca | 72162004 | 201 | 202 |
| Herpetosiphon | 159897776 | 203 | 204 |
| aurantiacus | | | |
| Streptomyces griseus | 182434863 | 205 | 206 |
| Mycobacterium | 120406242 | 207 | 208 |
| vanbaalenii | | | |
| Streptomyces | P50910 | 209 | 210 |
| diastaticus | | | |
| Deinococcus | 94972159 | 211 | 212 |
| geothermalis | | | |
| Arthrobacter sp. | A0JXN9 | 213 | 214 |
| Streptomyces | P24300 | 215 | 216 |
| rubiginosus | | | |
| Streptomyces murinus | P37031 | 217 | 218 |
| Thermus caldophilus | 4930285 | * | 219 |
| Thermus caldophilus | P56681 | * | 220 |
| Arthrobacter sp. | 231103 | * | 221 |
| Actinoplanes | 443486 | * | 222 |
| missouriensis | | | |
| Streptomyces | P15587 | * | 223 |
| olivochromogenes | | | |
| Streptomyces | 157879319 | * | 224 |
| olivochromogenes | | | |
| Streptomyces rochei | P22857 | * | 225 |
| Streptomyces | 157881044 | * | 226 |
| olivochromogenes | | | |
| Streptomyces | 7766813 | * | 227 |
| diastaticus | | | |
| Actinoplanes | 349936 | * | 228 |
| missouriensis | | | |
| Arthrobacter sp. | 2914276 | * | 229 |
| Streptomyces albus | P24299 | * | 230 |
| Actinoplanes | 443303 | * | 231 |
| missouriensis | | | |
| Streptomyces | 9256915 | * | 232 |
| diastaticus | | | |
| Actinoplanes | 443526 | * | 233 |
| missouriensis | | | |
| Streptomyces | 21730246 | * | 234 |
| rubiginosus | | | |
| Actinoplanes | 443568 | * | 235 |
| missouriensis | | | |

TABLE 5
SEQ ID numbers of Proteins xylose isomerases.
Uniprot accession number (AC) given for the indicated proteins.

| | | SEQ ID NO: Amino |
|---|---|---|
| Organism | GI or AC# | Acid |
| Salmonella enterica | B4T952 | 236 |
| Klebsiella pneumoniae | P29442 | 237 |
| Sinorhizobium meliloti | Q92LW9 | 238 |
| Escherichia coli | Q7A9X4 | 239 |
| Salmonella enterica | Q5PLM6 | 240 |
| Xanthomonas | Q3BMF2 | 241 |
| campestris | | |
| Pectobacterium | Q6DB05 | 242 |
| atrosepticum | | |
| Rhodopirellula baltica | Q7UVG2 | 243 |
| Xanthomonas | Q8PEW5 | 244 |
| axonopodis | | |
| Xanthomonas oryzae | Q5GUF2 | 245 |
| Pediococcus | Q03HN1 | 246 |
| pentosaceus | | |
| Brucella suis | Q8G204 | 247 |
| Escherichia coli | Q0TBN7 | 248 |
| Bifidobacterium longum | Q8G3Q1 | 249 |
| Brucella canis | A9M9H3 | 250 |
| Burkholderia | A9ARG7 | 251 |
| multivorans | | |
| Brucella ovis | A5VPA1 | 252 |
| Rhizobium etli | B3Q0R5 | 253 |
| Burkholderia | Q13RB8 | 254 |
| xenovorans | | |
| Actinobacillus | A3N3K2 | 255 |
| pleuropneumoniae | | |
| Burkholderia | B4ENA5 | 256 |
| cenocepacia | | |
| Solibacter usitatus | Q022S9 | 257 |
| Brucella abortus | B2SA37 | 258 |
| Rhodobacter | A4WVT8 | 259 |
| sphaeroides | | |
| Thermoanaerobacter | B0K1L3 | 260 |
| sp. | | |
| Yersinia | Q1C0D3 | 261 |
| pseudotuberculosis | | |
| Xanthomonas oryzae | Q5GYQ7 | 262 |
| Bifidobacterium longum | B3DR33 | 263 |
| Thermoanaerobacter | P22842 | 264 |
| pseudethanolicus | | |
| Photobacterium | Q6LUY7 | 265 |
| profundum | | |
| Escherichia coli | B1LJC7 | 266 |
| Agrobacterium | Q8U7G6 | 267 |
| tumefaciens | | |
| Tetragenococcus | O82845 | 268 |
| halophilus | | |
| Salmonella enterica | B4TZ55 | 269 |
| Yersinia | Q8Z9Z1 | 270 |
| pseudotuberculosis | | |
| Yersinia | Q1CDB8 | 271 |
| pseudotuberculosis | | |
| Rhodobacter | A3PNM4 | 272 |
| sphaeroides | | |
| Brucella abortus | Q2YMQ2 | 273 |
| Salmonella enterica | Q8ZL90 | 274 |
| Bacteroides vulgatus | A6L792 | 275 |
| Xanthomonas | Q8PLL9 | 276 |
| axonopodis | | |
| Salmonella enterica | Q57IG0 | 277 |
| Escherichia coli | B7M3I8 | 278 |
| Roseobacter | Q162B6 | 279 |
| denitrificans | | |
| Bacteroides fragilis | Q64U20 | 280 |
| Enterobacter sakazakii | A7MNI5 | 281 |
| Brucella abortus | Q57EI4 | 282 |
| Geobacillus | A4IP67 | 283 |
| thermodenitrificans | | |
| Bacteroides | Q8A9M2 | 284 |
| thetaiotaomicron | | |
| Haemophilus influenzae | A5UCZ3 | 285 |
| Yersinia | B2K7D2 | 286 |
| pseudotuberculosis | | |
| Xanthomonas | Q4UTU6 | 287 |
| campestris | | |
| Haemophilus somnus | B0UT19 | 288 |
| Pseudoalteromonas | Q15PG0 | 289 |
| atlantica | | |
| Escherichia fergusonii | B7LTH9 | 290 |
| Silicibacter sp. | Q1GKQ4 | 291 |
| Salmonella enterica | B5R4P8 | 292 |
| Bifidobacterium | A1A0H0 | 293 |
| adolescentis | | |
| Staphylococcus xylosus | P27157 | 294 |
| Thermotoga maritima | Q9X1Z5 | 295 |
| Salmonella enterica | A9MUV0 | 296 |
| Pseudomonas syringae | Q48J73 | 297 |
| Shigella boydii | Q31V53 | 298 |
| Burkholderia ambifaria | Q0B1U7 | 299 |
| Bacillus | A7Z522 | 300 |
| amyloliquefaciens | | |
| Haemophilus influenzae | A5UIN7 | 301 |
| Bacillus megaterium | O08325 | 302 |
| Arabidopsis thaliana | Q9FKK7 | 303 |
| Escherichia coli | Q3YVV0 | 304 |
| Bacteroides fragilis | Q5LCV9 | 305 |
| Pseudomonas | Q3KDW0 | 306 |
| fluorescens | | |
| Escherichia coli | B1X8I1 | 307 |
| Bacillus subtilis | P04788 | 308 |
| Xanthomonas | Q4UNZ4 | 309 |
| campestris | | |
| Pseudomonas syringae | Q4ZSF5 | 310 |
| Sinorhizobium medicae | A6UD89 | 311 |
| Ochrobactrum anthropi | A6X4G3 | 312 |
| Burkholderia | Q2SW40 | 313 |
| thailandensis | | |
| Salmonella enterica | B5EX72 | 314 |
| Thermotoga sp. | B1LB08 | 315 |
| Bacillus cereus | Q739D2 | 316 |
| Salmonella enterica | B4SWK9 | 317 |
| Salmonella enterica | Q7C637 | 318 |
| Enterococcus faecalis | Q7C3R3 | 319 |
| Thermotoga neapolitana | P45687 | 320 |
| Escherichia coli | B7MES1 | 321 |
| Photorhabdus | Q7N4P7 | 322 |
| luminescens | | |
| Enterobacter sp. | A4W566 | 323 |
| Burkholderia | B1KB47 | 324 |
| cenocepacia | | |
| Bacillus licheniformis | P77832 | 325 |
| Geobacillus | P54273 | 326 |
| stearothermophilus | | |
| Brucella abortus | Q8YFX5 | 327 |
| Rhizobium | Q1MBL8 | 328 |
| leguminosarum | | |
| Yersinia enterocolitica | A1JT10 | 329 |
| Serratia proteamaculans | A8G7W8 | 330 |
| Yersinia | A7FP68 | 331 |
| pseudotuberculosis | | |
| Escherichia coli | B7NEL7 | 332 |
| Yersinia pestis | A9R5Q1 | 333 |
| Fervidobacterium | Q6T6K9 | 334 |
| gondwanense | | |
| Xanthomonas | Q8P3H1 | 335 |
| campestris | | |
| Rhizobium | B5ZQV6 | 336 |
| leguminosarum | | |
| Bradyrhizobium | Q89VC7 | 337 |
| japonicum | | |
| Mesorhizobium sp. | Q11EH9 | 338 |
| Actinobacillus | B3H2X9 | 339 |
| pleuropneumoniae | | |
| Yersinia | Q663Y3 | 340 |
| pseudotuberculosis | | |
| Xanthomonas | Q8P9T9 | 341 |
| campestris | | |
| Burkholderia | A0KE56 | 342 |
| cenocepacia | | |
| Oceanobacillus | Q8ELU7 | 343 |
| iheyensis | | |
| Brucella suis | B0CKM9 | 344 |
| Thermoanaerobacterium | P29441 | 345 |
| thermosaccharolyticum | | |
| Burkholderia phymatum | B2JFE9 | 346 |
| Yersinia | B1JH40 | 347 |
| pseudotuberculosis | | |
| Bacillus sp. | P54272 | 348 |
| Lactococcus lactis | Q02Y75 | 349 |
| Novosphingobium | Q2GAB9 | 350 |
| aromaticivorans | | |
| Lactobacillus brevis | P29443 | 351 |
| Mesorhizobium loti | Q98CR8 | 352 |
| Escherichia coli | A8A623 | 353 |
| Burkholderia | Q1BG90 | 354 |
| cenocepacia | | |
| Thermoanaerobacterium | P19148 | 355 |
| thermosulfurigenes | | |
| Thermotoga petrophila | A5ILR5 | 356 |
| Lactobacillus pentosus | P21938 | 357 |
| Lactococcus lactis | Q9CFG7 | 358 |
| Ruminococcus | Q9S306 | 359 |
| flavefaciens | | |
| Burkholderia | B2T929 | 360 |
| phytofirmans | | |
| Salmonella enterica | B5FLD6 | 361 |
| Lactobacillus brevis | Q03TX3 | 362 |
| Burkholderia ambifaria | B1Z405 | 363 |
| Salmonella enterica | B5RGL6 | 364 |
| Bacillus halodurans | Q9K993 | 365 |
| Bacillus clausii | Q5WKJ3 | 366 |
| Marinomonas sp. | A6VWH1 | 367 |
| Yersinia | A4TS63 | 368 |
| pseudotuberculosis | | |
| Actinobacillus | B0BTI9 | 369 |
| pleuropneumoniae | | |
| Silicibacter pomeroyi | Q5LV46 | 370 |
| Xanthomonas oryzae | Q2NXR2 | 371 |
| Thermoanaerobacterium | P30435 | 372 |
| saccharolyticum | | |
| Escherichia coli | B6I3D6 | 373 |
| Escherichia coli | B5YVL8 | 374 |
| Escherichia coli | B7NP65 | 375 |
| Escherichia coli | B2U560 | 376 |
| Escherichia coli | B1IZM7 | 377 |
| Rhizobium etli | Q2K433 | 378 |
| Escherichia coli | P00944 | 379 |
| Hordeum vulgare | Q40082 | 380 |
| Dinoroseobacter shibae | A8LP53 | 381 |
| Rhodobacter | Q3IYM4 | 382 |
| sphaeroides | | |
| Actinobacillus | A6VLM8 | 383 |
| succinogenes | | |
| Bacillus pumilus | A8FE33 | 384 |
| Escherichia coli | Q8FCE3 | 385 |
| Pseudomonas syringae | Q880Z4 | 386 |
| Burkholderia | A4JSU5 | 387 |
| vietnamiensis | | |
| Escherichia coli | A7ZTB2 | 388 |
| Haemophilus influenzae | P44398 | 389 |
| Haemophilus influenzae | Q4QLI2 | 390 |
| Listeria welshimeri | A0AF79 | 391 |
| Thermoanaerobacter | Q9KGU2 | 392 |
| yonseiensis | | |
| Geobacillus | Q5KYS6 | 393 |
| kaustophilus | | |
| Mannheimia | Q65PY0 | 394 |
| succiniciproducens | | |

SEQ ID NO: 395 is the coding region for the Actinoplanes missourinesis xylose isomerase that was codon optimized for Zymomonas.

SEQ ID NO: 396 the coding region for the Lactobacillus brevis xylose isomerase that was codon optimized for Zymomonas.

SEQ ID NO: 397 is the coding region for the E. coli xylose isomerase that was codon optimized for Zymomonas.

SEQ ID NO: 398 is the nucleotide sequence of the codon optimized coding region for Geodermatophilus obscurus xylose isomerase.

SEQ ID NO: 399 is the nucleotide sequence of the codon optimized coding region for Mycobacterium smegmatis xylose isomerase.

SEQ ID NO: 74 is the nucleotide sequence of the codon optimized coding region for Salinispora arenicola xylose isomerase.

SEQ ID NO: 75 is the nucleotide sequence of the codon optimized coding region for Xylanimonas cellulosilytica xylose isomerase.

Other examples of polynucleotides, and polypeptides that can be used herein include, but are not limited to, polynucleotides and/or polypeptides having at least about 70% to about 75%, about 75% to about 80%, about 80% to about 85%, about 85% to about 90%, about 90% to about 95%, about 96%, about 97%, about 98%, or about 99% sequence identity to any one of the sequences of Tables 3, 4 or 5, wherein such a polynucleotide or gene encodes, or such a polypeptide has enzymatic activity. Still other examples of polynucleotides and polypeptides that can be used in the described isomerization and fermentation processes include, but are not limited to, an active variant, fragment or derivative of any one of the sequences of Tables 3, 4, or 5, wherein such a polynucleotide or gene encodes, or such a polypeptide has enzymatic activity.

In embodiments, the sequences of other polynucleotides and/or polypeptides can be identified in the literature and in bioinformatics databases well known to the skilled person using sequences disclosed herein and available in the art. For example, such sequences can be identified through BLAST searching of publicly available databases with known enzyme-encoding polynucleotide or polypeptide sequences. In such a method, identities can be based on the Clustal W method of alignment using the default parameters of GAP PENALTY=10, GAP LENGTH PENALTY=0.1, and Gonnet 250 series of protein weight matrix.

Additionally, the polynucleotide or polypeptide sequences disclosed herein or known in the art can be used to identify other homologs in nature. For example, each of the nucleic acid disclosed herein and fragments of the same can be used to isolate genes encoding homologous proteins. Isolation of homologous genes using sequence-dependent protocols is well known in the art. Examples of sequence-dependent protocols include, but are not limited to: (1) methods of nucleic acid hybridization; (2) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor et al., Proc. Acad. Sci. USA 82:1074 (1985); or strand displacement amplification (SDA), Walker et al., Proc. Natl. Acad. Sci. U.S.A., 89:392 (1992)]; and (3) methods of library construction and screening by complementation.

Methods for gene expression in recombinant host cells, including, but not limited to, yeast cells are known in the art (see, for example, Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Methods for gene expression by way of episomal plasmids and integrated polynucleotides are both compatible with the presently described methods.

In some embodiments, the coding region for the enzymes to be expressed can be codon optimized for the target host cell, as well known to one skilled in the art. Expression of genes in recombinant host cells, including but not limited to yeast cells, can require a promoter operably linked to a coding region of interest, and a transcriptional terminator. A number of promoters can be used in constructing expression cassettes for genes, including, but not limited to, the following constitutive promoters suitable for use in yeast: FBA1, TDH3, ADH1, and GPM1; and the following inducible promoters suitable for use in yeast: GAL1, GAL10 and CUP1. Suitable transcriptional terminators that can be used in a chimeric gene construct for expression include, but are not limited to, FBAlt, TDH3t, GPMlt, ERG10t, GALlt, CYClt, and ADHlt.

Recombinant polynucleotides are typically cloned for expression using the coding sequence as part of a chimeric gene used for transformation, which includes a promoter operably linked to the coding sequence and a termination control region. The coding region can be from the host cell for transformation and combined with regulatory sequences that are not native to the natural gene encoding the protein. Alternatively, the coding region can be from another host cell.

Vectors useful for the transformation of a variety of host cells are common and described in the literature. Typically the vector contains a selectable marker and sequences allowing autonomous replication or chromosomal integration in the desired host. In addition, suitable vectors can comprise a promoter region which harbors transcriptional initiation controls and a transcriptional termination control region, between which a coding region DNA fragment can be inserted, to provide expression of the inserted coding region. Both control regions can be derived from genes homologous to the transformed host cell, although it is to be understood that such control regions can also be derived from genes that are not native to the specific species chosen as a production host.

In embodiments, suitable promoters, transcriptional terminators, and enzyme coding regions can be cloned into E. coli-yeast shuttle vectors, and transformed into yeast cells. Such vectors allow strain propagation in both E. coli and yeast strains, and can contain a selectable marker and sequences allowing autonomous replication or chromosomal integration in the desired host. Typically used plasmids in yeast include, but are not limited to, shuttle vectors pRS423, pRS424, pRS425, and pRS426 (American Type Culture Collection, Rockville, Md.), which contain an E. coli replication origin (e.g., pMB1), a yeast 2-micron origin of replication, and a marker for nutritional selection. The selection markers for these four vectors are HIS3 (vector pRS423), TRP1 (vector pRS424), LEU2 (vector pRS425) and URA3 (vector pRS426).

In embodiments, construction of expression vectors with a chimeric gene encoding the described enzyme can be performed by the gap repair recombination method in yeast. The gap repair cloning approach takes advantage of the highly efficient homologous recombination system in yeast. In embodiments, a yeast vector DNA is digested (e.g., in its multiple cloning site) to create a “gap” in its sequence. A number of insert DNAs of interest are generated that contain an approximately 21 bp sequence at both the 5′ and the 3′ ends that sequentially overlap with each other, and with the 5′ and 3′ terminus of the vector DNA. For example, to construct a yeast expression vector for “Gene X,” a yeast promoter and a yeast terminator are selected for the expression cassette. The promoter and terminator are amplified from the yeast genomic DNA, and Gene X is either PCR amplified from its source organism or obtained from a cloning vector comprising Gene X sequence. There is at least a 21 bp overlapping sequence between the 5′ end of the linearized vector and the promoter sequence, between the promoter and Gene X, between Gene X and the terminator sequence, and between the terminator and the 3′ end of the linearized vector. The “gapped” vector and the insert DNAs are then co-transformed into a yeast strain and plated on the medium containing the appropriate compound mixtures that allow complementation of the nutritional selection markers on the plasmids. The presence of correct insert combinations can be confirmed by PCR mapping using plasmid DNA prepared from the selected cells. The plasmid DNA isolated from yeast (usually low in concentration) can then be transformed into an E. coli strain, e.g. TOP10, followed by mini preps and restriction mapping to further verify the plasmid construct. Finally the construct can be verified by DNA sequence analysis.

Like the gap repair technique, integration into the yeast genome also takes advantage of the homologous recombination system in yeast. In embodiments, a cassette containing a coding region plus control elements (promoter and terminator) and auxotrophic marker is PCR-amplified with a high-fidelity DNA polymerase using primers that hybridize to the cassette and contain 40-70 base pairs of sequence homology to the regions 5′ and 3′ of the genomic locus where insertion is desired. The PCR product is then transformed into yeast and plated on medium containing the appropriate compound mixtures that allow selection for the integrated auxotrophic marker. For example, to integrate “Gene X” into chromosomal location “Y,” the promoter-coding region X-terminator construct is PCR amplified from a plasmid DNA construct and joined to an autotrophic marker (such as URA3) by either SOE PCR or by common restriction digests and cloning. The full cassette, containing the promoter-coding regionX-terminator-URA3 region, is PCR amplified with primer sequences that contain 40-70 bp of homology to the regions 5′ and 3′ of location “Y” on the yeast chromosome. The PCR product is transformed into yeast and selected on growth media lacking uracil. Transformants can be verified either by colony PCR or by direct sequencing of chromosomal DNA.

The presence of xylulose-producing enzymatic activity (e.g., xylose isomerase, xylulose kinases, etc) in the recombinant host cells disclosed herein can be confirmed using methods known in the art. In a non-limiting example, transformants can be screened by PCR using primers for the enzyme. In another non-limiting example, enzymatic activity can be assayed for in a recombinant host cell disclosed herein that lacks the enzymatic activity endogenously. For example, a polypeptide having enzymatic activity can convert xylose or arabinose into xylulose. In another non-limiting example, enzymatic activity can be confirmed by more indirect methods, such as by assaying for a downstream product in a pathway requiring the enzymatic activity, including, for example, isobutanol production.

Improving xylulose production by metabolic engineering will enable isobutanol production by fermentation in the absence of exogenous enzymes. Thus, fermentation of 5-carbon sugars to butanol can be improved by adding exogenous enzymes, by recombinantly expressing enzymes, or both. With increased xylulose production, the efficacy of producing isobutanol from 5-carbon sugars is increased.

In some embodiments, the use of enzymes that convert substrates to xylulose results in a particular xylose: xylulose equilibrium. For example, in some embodiments, the equilibrium is about 5 xylose: 1 xylulose.

In some embodiments, the enzymes are present in an amount sufficient to convert a substrate to xylulose at a rate of at least about 0.1 g/hour, at least about 0.25 g/hour, at least about 0.5 g/hour, or at least about 1 g/hour.

The use of enzymes that convert substrates to xylulose allow for increased production of butanol.

In some embodiments, the use of such enzymes results in an increase in the consumption of 5-carbon sugars. The rate of consumption of 5-carbon sugars can be measured using any means known in the art. In certain embodiments, in which 6-carbon sugars are also consumed, the rate of consumption of 5-carbon sugars can be at least about 0.5%, 0.75%, 1%, 2%, 3%, 4%, 5%, 10%, 20%, 30%, 40%, 50%, 60%, or 70% the rate of consumption of 6-carbon sugars.

In some embodiments, the microorganisms capable of producing butanol are genetically stable. Chromosomal aberrations and plasmid loss are minimized in genetically stable microorganisms. In some embodiments, the microorganisms capable of producing butanol are genetically stable when grown in industrially relevant cultivation media. In some embodiments, the microorganisms capable of producing butanol are genetically stable when grown in mineral medium. In some embodiments, the microorganisms capable of producing butanol are genetically stable when grown in defined medium. In some embodiments, the microorganisms capable of producing butanol are genetically stable over periods of prolonged continuous culture.

### Isomerization and Fermentation

Butanol-producing microorganisms can be cultured under any conditions that allow for butanol production. In particular, it has been observed that growth of the microorganism in the presence of aeration followed by fermentation in the absence of respiration increases butanol production (anaerobic or microaerobic fermentation).

Respiration can be measured using any means known in the art. By way of example, respiration can be assessed by ATP production, carbon dioxide production, and/or oxygen use. Respiration can be inhibited by any means known in the art. For example, inhibitors of respiration can be added to the fermenting composition. Suitable inhibitors of respiration include, by way of example, Antimycin A, cyanide, azide, oligomycin, and rotenone.

The inhibitor can be present at any concentration that decreases or limits respiration. In some embodiments, the inhibitor is present at a concentration of about 0.1 to about 10 μM. For example, the concentration of the inhibitor can be about 0.1 to about μM, about 0.1 to about 4 μM, about 0.1 to about 3 μM, about 0.1 to about 2 μM, about 0.1 to about 1.5 μM, or about 0.1 to about 1 μM. The concentration of the inhibitor can also be about 0.5 to about 10 μM, about 0.5 to about 5 μM, about 0.5 to about 3 μM, about 0.5 to about 2 μM, about 0.5 to about 1.5 μM, or about 0.5 to about 1 μM. The concentration of the inhibitor can also be about 1 μM.

The inhibitor can be present at a concentration that is sufficient to reduce respiration to a level that is no more than about 1%, 5%, 10%, 20%, 30%, 40%, 50%, 60%, or 75% of the level of respiration under the same conditions in the absence of the inhibitor.

In some embodiments, the inhibitor of respiration is Antimycin A. In some embodiments, the Antimycin A is present at a concentration of about 0.1 to about 10 μM. For example, the concentration of the Antimycin A can be about 0.1 to about 5 μM, about 0.1 to about 4 μM, about 0.1 to about 3 μM, about 0.1 to about 2 μM, about 0.1 to about 1.5 μM, or about 0.1 to about 1 μM. The concentration of the Antimycin A can also be about 0.5 to about 10 μM, about 0.5 to about 5 μM, about 0.5 to about 3 μM, about 0.5 to about 2 μM, about 0.5 to about 1.5 μM, or about 0.5 to about 1 μM. The concentration of the Antimycin A can also be about 1 μM.

The Antimycin A can be present at a concentration that is sufficient to reduce respiration to a level that is no more than about 1%, 5%, 10%, 20%, 30%, 40%, 50%, 60%, or 75% of the level of respiration under the same conditions in the absence of the Antimycin A.

In some embodiments, the culture conditions are such that the fermentation occurs without respiration in the absence of inhibitors. For example, cells can be cultured in a fermenter under micro-aerobic or anaerobic conditions.

Other conditions that maximize butanol production can also be provided.

Typically microorganisms are grown at a temperature in the range of about 20° C. to about 40° C. Conversion of 5-carbon sugars into xylulose (isomerization) and fermentation can be performed at the same or different temperatures. For example, temperatures of about 40° C. can be used for the conversion of 5-carbon sugars into xylulose, and temperatures of about 30° C. can be used for the fermentation of xylulose to butanol. Furthermore, temperatures of about 30° C. to about 40° C., about 31° C. to about 39° C., about 32° C. to about 38° C., about 32° C. to about 37° C., about 33° C. to about 36° C., or about 33° C. to about 35° C. can be used for both conversion of 5-carbon sugars into xylulose and fermentation of xylulose to butanol. In addition, temperatures of about 32° C. to about 36° C., about 32° C. to about 35° C., about 32° G to about 34° C., about 33° C. to about 36° C., about 33° C. to about 35° C., or about 33° C. to about 34° C. can be used for both conversion of 5-carbon sugars into xylulose and fermentation of xylulose to butanol. In addition, temperatures of about 32° C. to about 36° C., about 33° C. to about 36° C., about 34° C. to about 36° C., about 33° C. to about 35° C., about 33° C. to about 35° C., or about 34° C. to about 35° C. can be used for both conversion of 5-carbon sugars into xylulose and fermentation of xylulose to butanol. In some embodiments, a temperature of about 33° C. to about 35° C. or a temperature of about 34° C. is used to convert 5-carbon sugars to xylulose and to ferment xylulose to butanol.

Suitable pH ranges for the microorganisms are about pH 3.0 to about pH 9.0. Conversion of 5-carbon sugars into xylulose (isomerization) and fermentation can be performed at the same or different pH. In some embodiments, the isomerization occurs at a pH of about pH 5.0 to about pH 8.0, about pH 5.0 to about pH 7.0, about pH 6.0 to about pH 8.0, about pH 6.0 to about pH 7.0, or about 7.0. In some embodiments, the fermentation occurs at a pH of about pH 3.0 to about pH 7.0, about pH 4.0 to about pH 6.0, about pH 4.0 to about pH 5.0.

In some embodiments, isomerization and fermentation occur at a pH of about pH 4.0 to about pH 8.0, about pH 5.0 to about pH 7.0, or about pH 6.0. In some embodiments, isomerization and fermentation occur at a pH of about pH 5.0 to about pH 8.0, or about pH 6.0 to about pH 8.0. In some embodiments, isomerization and fermentation occur at a pH that is about pH 4.0 to about pH 7.0, about pH 4.0 to about pH 6.0. In some embodiments, isomerization and fermentation occur at a pH that is about pH 6.0.

In addition, fermentation media must contain suitable minerals, salts, cofactors, buffers and other components, known to those skilled in the art, suitable for the growth of the cultures and promotion of an enzymatic pathway described herein. Non-limiting examples of media that can be used include yeast extract-peptone, a defined mineral medium, yeast nitrogen base (YNB), synthetic complete (SC), M122C, MOPS, SOB, TSY, YMG, YPD, 2XYT, LB, M17, or M9 minimal media. Other examples of media that can be used include solutions containing potassium phosphate and/or sodium phosphate. Suitable media can be supplemented with NADH or NADPH. Other suitable growth media in the present invention are common commercially prepared media such as Luria Bertani (LB) broth, Sabouraud Dextrose (SD) broth, Yeast Medium (YM) broth, or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media can also be used, and the appropriate medium for growth of the particular microorganism will be known by one skilled in the art of microbiology or fermentation science.

In some embodiments, the fermentation media does not contain yeast extract.

In some embodiments, antibiotics are included. For example, methods which use an exogenous source of an xylulose-producing enzyme can introduce bacterial contaminants. For example, antibiotics such as Penicillins (e.g., Penicillin G or Penicillin V), Tetracyclines, or Cephalosporins (e.g., Cephalosporin C), virginiamycin, and chloramphenicol can be used. In some embodiments, the antibiotic is present in an amount sufficient to inhibit bacterial growth. In some embodiments, the antibiotic is present in an amount that does not affect yeast growth. In some embodiments, the antibiotic is present at a concentration of about 5, 10, 15, 20, 25, 30, 35, 40, 45, or 50 μg/L.

In some embodiments, the compositions are cultured for at least about 20 hours, at least about 30 hours, at least about 40 hours, at least about 50 hours, at least about 60 hours, at least about 70 hours, at least about 80 hours, at least about 90 hours, at least about 100 hours, at least about 120 hours, at least about 140 hours, at least about 160 hours, at least about 180 hours, or at least about 200 hours.

It is contemplated that the production of isobutanol, or other products, can be practiced using batch, fed-batch or continuous processes and that any known mode of fermentation would be suitable. Additionally, it is contemplated that cells can be immobilized on a substrate or in a matrix as whole cell catalysts and subjected to fermentation conditions for isobutanol production.

### Isobutanol Production

Methods for the production of butanol using 5-carbon sugars are described herein. In some embodiments, the butanol is isobutanol.

For example, butanol can be produced from 5-carbon sugars by (a) providing a composition comprising a microorganism capable of producing butanol and an enzyme or combination of enzymes capable of converting a substrate to xylulose; (b) contacting the composition with a source of 5-carbon sugars; and (c) culturing the yeast under conditions that limits yeast respiration.

Thus, compositions for producing butanol from 5-carbon sugars are also provided. The compositions comprise (a) a yeast capable of producing butanol; (b) an enzyme or combination of enzymes capable of converting a 5-carbon sugar to xylulose; (c) a source of 5-carbon sugars; and (d) a fermentation media.

In some embodiments, the butanol or isobutanol is produced at a particular yield or rate.

Thus, the specific isobutanol production rate can be at least about 0.10 g/g/h (grams of isobutanol per gram dry cell weight per hour), at least about 0.11 g/g/h, at least about 0.12 g/g/h, at least about 0.13 g/g/h, at least about 0.14 g/g/h, at least about 0.15 g/g/h, at least about 0.16 g/g/h, at least about 0.17 g/g/h, at least about 0.18 g/g/h, at least about 0.19 g/g/h, at least about 0.20 g/g/h, at least about 0.25 g/g/h, at least about 0.30 g/g/h, at least about 0.35 g/g/h, at least about 0.40 g/g/h, at least about 0.45 g/g/h, at least about 0.50 g/g/h, at least about 0.75 g/g/hr, or at least about 1.0 g/g/hr. The specific isobutanol production rate can also be about 0.05 g/g/h to about 1.0 g/g/h, about 0.05 g/g/h to about 0.75 g/g/h, or about 0.05 g/g/h to about 0.50 g/g/h. The specific isobutanol production rate can also be about 0.10 g/g/h to about 1.0 g/g/h, about 0.10 g/g/h to about 0.75 g/g/h, or about 0.10 to about 0.50 g/g/h. The specific isobutanol production rate can also be about 0.15 g/g/h to about 1.0 g/g/h, about 0.15 g/g/h to about 0.75 g/g/h, or about 0.15 g/g/h to about 0.5 g/g/h.

In certain embodiments, the production provides a yield of greater than about 10% of theoretical, at a yield of greater than about 20% of theoretical, at a yield of greater than about 25% of theoretical, at a yield of greater than about 30% of theoretical, at a yield of greater than about 40% of theoretical, at a yield of greater than about 50% of theoretical, at a yield of greater than about 60% of theoretical, at a yield of greater than about 70% of theoretical, at a yield of greater than about 75% of theoretical, at a yield of greater than about 80% of theoretical at a yield of greater than about 85% of theoretical, at a yield of greater than about 90% of theoretical, at a yield of greater than about 95% of theoretical, at a yield of greater than about 96% of theoretical, at a yield of greater than about 97% of theoretical, at a yield of greater than about 98% of theoretical, at a yield of greater than about 99% of theoretical, or at a yield of about 100% of theoretical.

In certain embodiments, where both isobutanol and ethanol are produced, the rate of isobutanol production can be at and the rate of isobutanol will decrease in the presence of ethanol production.

### Microorganisms

According to the methods described herein, any microorganism capable of producing butanol can be used. For example, in some embodiments, the microorganism is a yeast cell capable of producing butanol. In some embodiments, the yeast cell is a member of a genus selected from the group consisting of: Saccharomyces, Schizosaccharomyces, Hansenula, Candida, Kluyveromyces, Yarrowia, Issatchenkia, and Pichia. In still another aspect, the yeast cell is Saccharomyces cerevisiae.

The microorganism can be genetically altered in order to allow it to produce butanol. Biosynthetic pathways for the production of isobutanol that may be used include those described in U.S. Pat. No. 7,993,889, which is incorporated herein by reference. For example, the microorganism capable of producing butanol can comprise a polynucleotide that encodes a polypeptide that catalyzes the conversion of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; or (e) isobutyraldehyde to isobutanol. In some embodiments, the microorganism comprises polynucleotides that encode polypeptides that catalyzes the conversion of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the microorganism comprises polynucleotides encoding polypeptides having acetolactate synthase, keto acid reductoisomerase, dihydroxy acid dehydratase, ketoisovalerate decarboxylase, and/or alcohol dehydrogenase activity.

The microorganism capable of producing butanol can comprise a polynucleotide that encodes a polypeptide that catalyzes the conversion of: (a) pyruvate to acetolactate; (b) acetolactate to 2,3-dihydroxyisovalerate; (c) 2,3-dihydroxyisovalerate to α-ketoisovalerate; (d) α-ketoisovalerate to valine; (e) valine to isobutylamine; (f) isobutylamine to isobutyraldehyde, (g) isobutyraldehyde to isobutanol. In some embodiments, the microorganism comprises polynucleotides that encode polypeptides that catalyzes the conversion of: (a) pyruvate to acetolactate; (b) acetolactate to 2,3-dihydroxyisovalerate; (c) 2,3-dihydroxyisovalerate to α-ketoisovalerate; (d) α-ketoisovalerate to valine; (e) valine to isobutylamine; (f) isobutylamine to isobutyraldehyde, (g) isobutyraldehyde to isobutanol. In some embodiments, the microorganism comprises polynucleotides encoding polypeptides having acetolactate synthase, ketol-acid reductoisomerase, dihydroxyacid dehydratase, transaminase, valine dehydrogenase, valine decarboxylase, omega transaminase, and/or branched-chain alcohol dehydrogenase activity.

The microorganism capable of producing butanol can comprise a polynucleotide that encodes a polypeptide that catalyzes the conversion of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyryl-CoA; (e) isobutyryl-CoA to isobutyraldehyde; and (f) isobutyraldehyde to isobutanol. In some embodiments, the microorganism comprises polynucleotides that encode polypeptides that catalyzes the conversion of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyryl-CoA; (e) isobutyryl-CoA to isobutyraldehyde; and (f) isobutyraldehyde to isobutanol. In some embodiments, the microorganism comprises polynucleotides encoding polypeptides having acetolactate synthase, acetohydroxy acid reductoisomerase, acetohydroxy acid dehydratase, branched-chain keto acid dehydrogenase, acetylating aldehyde dehydrogenase, and/or branched-chain alcohol dehydrogenase activity.

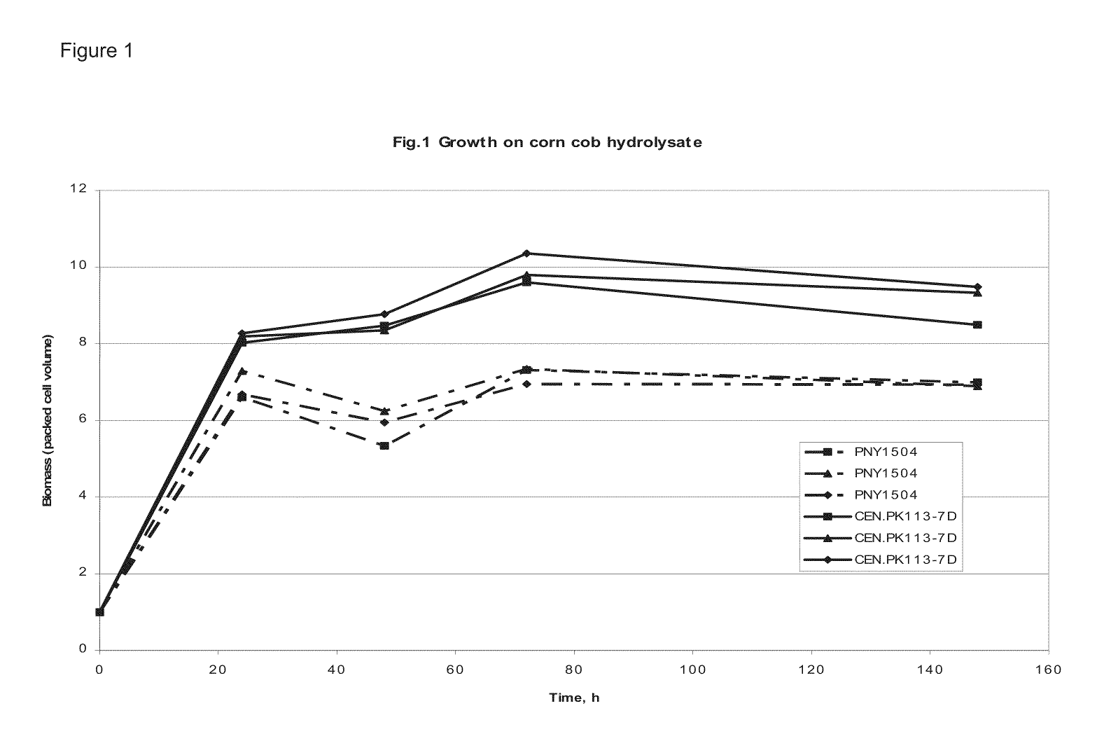

The microorganism capable of producing butanol can comprise a polynucleotide that encodes a polypeptide that catalyzes the conversion (a) butyryl-CoA to isobutyryl-CoA, (b) isobutyryl-CoA to isobutyraldehyde; and (c) isobutyraldehyde to isobutanol. In some embodiments, the microorganism comprises polynucleotides that encode polypeptides that catalyzes the conversion of: (a) butyryl-CoA to isobutyryl-CoA, (b) isobutyryl-CoA to isobutyraldehyde; and (c) isobutyraldehyde to isobutanol. In some embodiments, the microorganism comprises polynucleotides encoding polypeptides having isobutyryl-CoA mutase, acetylating aldehyde dehydrogenase, and/or branched-chain alcohol dehydrogenase activity, as described in steps k, e, and g in FIG. 1 from U.S. Pat. No. 7,993,889, which is herein incorporated by reference

Biosynthetic pathways for the production of 1-butanol that may be used include those described in U.S. Appl. Pub. No. 2008/0182308, which is incorporated herein by reference. The microorganism capable of producing butanol can comprise a polynucleotide that encodes a polypeptide that catalyzes the conversion of: (a) acetyl-CoA to acetoacetyl-CoA; (b) acetoacetyl-CoA to 3-hydroxybutyryl-CoA; (c) 3-hydroxybutyryl-CoA to crotonyl-CoA; (d) crotonyl-CoA to butyryl-CoA; (e) butyryl-CoA to butyraldehyde; and (f) butyraldehyde to 1-butanol. In some embodiments, the microorganism comprises polynucleotides that encode polypeptides that catalyzes the conversion of: (a) acetyl-CoA to acetoacetyl-CoA; (b) acetoacetyl-CoA to 3-hydroxybutyryl-CoA; (c) 3-hydroxybutyryl-CoA to crotonyl-CoA; (d) crotonyl-CoA to butyryl-CoA; (e) butyryl-CoA to butyraldehyde; and (f) butyraldehyde to 1-butanol. In some embodiments, the microorganism comprises polynucleotides encoding polypeptides having acetyl-CoA acetyl transferase, 3-hydroxybutyryl-CoA dehydrogenase, crotonase, butyryl-CoA dehydrogenase, butyraldehyde dehydrogenase, and/or butanol dehydrogenase activity.

Biosynthetic pathways for the production of 2-butanol that may be used include those described in U.S. Appl. Pub. No. 2007/0259410 and U.S. Appl. Pub. No. 2009/0155870, which are incorporated herein by reference. The microorganism capable of producing butanol can comprise a polynucleotide that encodes a polypeptide that catalyzes the conversion of: (a) pyruvate to alpha-acetolactate; (b) alpha-acetolactate to acetoin; (c) acetoin to 3-amino-2-butanol; (d) 3-amino-2-butanol to 3-amino-2-butanol phosphate; (e) 3-amino-2-butanol phosphate to 2-butanone; and (f) 2-butanone to 2-butanol. In some embodiments, the microorganism comprises polynucleotides that encode polypeptides that catalyzes the conversion of: (a) pyruvate to alpha-acetolactate; (b) alpha-acetolactate to acetoin; (c) acetoin to 3-amino-2-butanol; (d) 3-amino-2-butanol to 3-amino-2-butanol phosphate; (e) 3-amino-2-butanol phosphate to 2-butanone; and (f) 2-butanone to 2-butanol. In some embodiments, the microorganism comprises polynucleotides encoding polypeptides having acetolactate synthase, acetolactate decarboxylase, acetonin aminase, aminobutanol kinase, aminobutanol phosphate phosphorylase, and/or butanol dehydrogenase activity.

The microorganism capable of producing butanol can comprise a polynucleotide that encodes a polypeptide that catalyzes the conversion of: (a) pyruvate to alpha-acetolactate; (b) alpha-acetolactate to acetoin; (c) acetoin to 2,3-butanediol; (d) 2,3-butanediol to 2-butanone; and (e) 2-butanone to 2-butanol. In some embodiments, the microorganism comprises polynucleotides that encode polypeptides that catalyzes the conversion of: (a) pyruvate to alpha-acetolactate; (b) alpha-acetolactate to acetoin; (c) acetoin to 2,3-butanediol; (d) 2,3-butanediol to 2-butanone; and (e) 2-butanone to 2-butanol. In some embodiments, the microorganism comprises polynucleotides encoding polypeptides having acetolactate synthase, acetolactate decarboxylase, butanediol dehydrogenase, dial dehydratase, and/or butanol dehydrogenase activity.

Biosynthetic pathways for the production of 2-butanone that may be used include those described in U.S. Appl. Pub. No. 2007/0259410 and U.S. Appl. Pub. No. 2009/0155870, which are incorporated herein by reference. The microorganism capable of producing butanol can comprise a polynucleotide that encodes a polypeptide that catalyzes the conversion of: (a) pyruvate to alpha-acetolactate; (b) alpha-acetolactate to acetoin; (c) acetoin to 3-amino-2-butanol; (d) 3-amino-2-butanol to 3-amino-2-butanol phosphate; and (e) 3-amino-2-butanol phosphate to 2-butanone. In some embodiments, the microorganism comprises polynucleotides that encode polypeptides that catalyzes the conversion of: (a) pyruvate to alpha-acetolactate; (b) alpha-acetolactate to acetoin; (c) acetoin to 3-amino-2-butanol; (d) 3-amino-2-butanol to 3-amino-2-butanol phosphate; and (e) 3-amino-2-butanol phosphate to 2-butanone. In some embodiments, the microorganism comprises polynucleotides encoding polypeptides having acetolactate synthase, acetolactate decarboxylase, acetonin aminase, aminobutanol kinase, and/or aminobutanol phosphate phosphorylase activity.

The microorganism capable of producing butanol can comprise a polynucleotide that encodes a polypeptide that catalyzes the conversion of: (a) pyruvate to alpha-acetolactate; (b) alpha-acetolactate to acetoin; (c) acetoin to 2,3-butanediol; and (d) 2,3-butanediol to 2-butanone. In some embodiments, the microorganism comprises polynucleotides that encode polypeptides that catalyzes the conversion of: (a) pyruvate to alpha-acetolactate; (b) alpha-acetolactate to acetoin; (c) acetoin to 2,3-butanediol; and (d) 2,3-butanediol to 2-butanone. In some embodiments, the microorganism comprises polynucleotides encoding polypeptides having acetolactate synthase, acetolactate decarboxylase, butanediol dehydrogenase, and/or dial dehydratase activity.

In addition, in some embodiments, the microorganism comprises at least one deletion, mutation, and/or substitution in an endogenous polynucleotide encoding a polypeptide having pyruvate decarboxylase activity. The polypeptide having pyruvate decarboxylase activity can be, by way of example, Pdc1, Pdc5, Pdc6, or any combination thereof. In some embodiments, the microorganism is substantially free of an enzyme having pyruvate decarboxylase activity. A genetic modification which has the effect of reducing glucose repression wherein the yeast production host cell is pdc- is described in U.S. Appl. Publication No. 20110124060, incorporated herein by reference.

It will be appreciated that microorganisms comprising a butanol biosynthetic pathway as provided herein may further comprise one or more additional modifications. U.S. Appl. Pub. No. 20090305363 (incorporated herein by reference) discloses increased conversion of pyruvate to acetolactate by engineering yeast for expression of a cytosol-localized acetolactate synthase and substantial elimination of pyruvate decarboxylase activity. In some embodiments, the host cells comprise modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Patent Appl. Pub. No. 20090305363 (incorporated herein by reference), modifications to a host cell that provide for increased carbon flux through an Entner-Doudoroff Pathway or reducing equivalents balance as described in U.S. Patent Appl. Pub. No. 20100120105 (incorporated herein by reference). Other modifications include integration of at least one polynucleotide encoding a polypeptide that catalyzes a step in a pyruvate-utilizing biosynthetic pathway. Other modifications include at least one deletion, mutation, and/or substitution in an endogenous polynucleotide encoding a polypeptide having acetolactate reductase activity. In embodiments, the polypeptide having acetolactate reductase activity is YMR226c of Saccharomyces cerevisae or a homolog thereof. Additional modifications include a deletion, mutation, and/or substitution in an endogenous polynucleotide encoding a polypeptide having aldehyde dehydrogenase and/or aldehyde oxidase activity. In embodiments, the polypeptide having aldehyde dehydrogenase activity is ALD6 from Saccharomyces cerevisiae or a homolog thereof. In some embodiments, microorganisms contain a deletion or downregulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

In some embodiments, the yeast strain is PNY1504. PNY1504 was derived from CEN.PK 113-7D (CBS 8340; Centraalbureau voor Schimmelcultures (CBS) Fungal Biodiversity Centre, Netherlands) and contains deletions of the following genes: URA3, HIS3, PDC1, PDC5, PDC6, and GPD2. This strain was transformed with plasmids pYZ090 (SEQ ID NO: 1) and pLH468 (SEQ ID NO: 2) to create strain PNY1504 (BP1083, NGC1-070). Plasmids pYZ090 and pLH468 were described in U.S. Provisional Application No. 61/246,844, which is hereby incorporated by reference in its entirety. In some embodiments, the microorganism comprises a polynucleotide encoding one or more polypeptides that function in the pentose phosphate pathway. For example, the polypeptide can be a transketolase, a transaldolase, a ribulose-phosphate 3-epimerase, and/or a ribose-5-phosphate isomerase. Sequences of exemplary pentose phosphate pathway proteins are found in Table 6 below.

TABLE 6
Pentose Phosphate Pathway Enzymes.
Table 6: Pentose Phosphate Pathway Enzymes
Genomic coding region sequence records from Saccharomyces
Genome Database are shown in FASTA format.

| | EC | |
|---|---|---|
| Enzyme | Number | SEQ ID NO |
| Transketolase | 2.2.1.1 | >TKL1 YPR074C Chr 16 |
| | | ATGACTCAATTCACTGACATTGATAAGCTAGCCGTCTCCACC |
| | | ATAAGAATTTTGGCTGTGGACACCGTATCCAAGGCCAACTC |
| | | AGGTCACCCAGGTGCTCCATTGGGTATGGCACCAGCTGCAC |
| | | ACGTTCTATGGAGTCAAATGCGCATGAACCCAACCAACCCA |
| | | GACTGGATCAACAGAGATAGATTTGTCTTGTCTAACGGTCA |
| | | CGCGGTCGCTTTGTTGTATTCTATGCTACATTTGACTGGTTA |
| | | CGATCTGTCTATTGAAGACTTGAAACAGTTCAGACAGTTGG |
| | | GTTCCAGAACACCAGGTCATCCTGAATTTGAGTTGCCAGGT |
| | | GTTGAAGTTACTACCGGTCCATTAGGTCAAGGTATCTCCAAC |
| | | GCTGTTGGTATGGCCATGGCTCAAGCTAACCTGGCTGCCACT |
| | | TACAACAAGCCGGGCTTTACCTTGTCTGACAACTACACCTAT |
| | | GTTTTCTTGGGTGACGGTTGTTTGCAAGAAGGTATTTCTTCA |
| | | GAAGCTTCCTCCTTGGCTGGTCATTTGAAATTGGGTAACTTG |
| | | ATTGCCATCTACGATGACAACAAGATCACTATCGATGGTGC |
| | | TACCAGTATCTCATTCGATGAAGATGTTGCTAAGAGATACG |
| | | AAGCCTACGGTTGGGAAGTTTTGTACGTAGAAAATGGTAAC |
| | | GAAGATCTAGCCGGTATTGCCAAGGCTATTGCTCAAGCTAA |
| | | GTTATCCAAGGACAAACCAACTTTGATCAAAATGACCACAA |
| | | CCATTGGTTACGGTTCCTTGCATGCCGGCTCTCACTCTGTGC |
| | | ACGGTGCCCCATTGAAAGCAGATGATGTTAAACAACTAAAG |
| | | AGCAAATTCGGTTTCAACCCAGACAAGTCCTTTGTTGTTCCA |
| | | CAAGAAGTTTACGACCACTACCAAAAGACAATTTTAAAGCC |
| | | AGGTGTCGAAGCCAACAACAAGTGGAACAAGTTGTTCAGCG |
| | | AATACCAAAAGAAATTCCCAGAATTAGGTGCTGAATTGGCT |
| | | AGAAGATTGAGCGGCCAACTACCCGCAAATTGGGAATCTAA |
| | | GTTGCCAACTTACACCGCCAAGGACTCTGCCGTGGCCACTA |
| | | GAAAATTATCAGAAACTGTTCTTGAGGATGTTTACAATCAA |
| | | TTGCCAGAGTTGATTGGTGGTTCTGCCGATTTAACACCTTCT |
| | | AACTTGACCAGATGGAAGGAAGCCCTTGACTTCCAACCTCC |
| | | TTCTTCCGGTTCAGGTAACTACTCTGGTAGATACATTAGGTA |
| | | CGGTATTAGAGAACACGCTATGGGTGCCATAATGAACGGTA |
| | | TTTCAGCTTTCGGTGCCAACTACAAACCATACGGTGGTACTT |
| | | TCTTGAACTTCGTTTCTTATGCTGCTGGTGCCGTTAGATTGTC |
| | | CGCTTTGTCTGGCCACCCAGTTATTTGGGTTGCTACACATGA |
| | | CTCTATCGGTGTCGGTGAAGATGGTCCAACACATCAACCTA |
| | | TTGAAACTTTAGCACACTTCAGATCCCTACCAAACATTCAAG |
| | | TTTGGAGACCAGCTGATGGTAACGAAGTTTCTGCCGCCTAC |
| | | AAGAACTCTTTAGAATCCAAGCATACTCCAAGTATCATTGCT |
| | | TTGTCCAGACAAAACTTGCCACAATTGGAAGGTAGCTCTAT |
| | | TGAAAGCGCTTCTAAGGGTGGTTACGTACTACAAGATGTTG |
| | | CTAACCCAGATATTATTTTAGTGGCTACTGGTTCCGAAGTGT |
| | | CTTTGAGTGTTGAAGCTGCTAAGACTTTGGCCGCAAAGAAC |
| | | ATCAAGGCTCGTGTTGTTTCTCTACCAGATTTCTTCACTTTTG |
| | | ACAAACAACCCCTAGAATACAGACTATCAGTCTTACCAGAC |
| | | AACGTTCCAATCATGTCTGTTGAAGTTTTGGCTACCACATGT |
| | | TGGGGCAAATACGCTCATCAATCCTTCGGTATTGACAGATTT |
| | | GGTGCCTCCGGTAAGGCACCAGAAGTCTTCAAGTTCTTCGG |
| | | TTTCACCCCAGAAGGTGTTGCTGAAAGAGCTCAAAAGACCA |
| | | TTGCATTCTATAAGGGTGACAAGCTAATTTCTCCTTTGAAAA |
| | | AAGCTTTCTAA |
| | | (SEQ ID NO: 85) |
| Transaldolase | 2.2.1.2 | >TAL1 YLR354C Chr 12 |
| | | ATGTCTGAACCAGCTCAAAAGAAACAAAAGGTTGCTAACAA |
| | | CTCTCTAGAACAATTGAAAGCCTCCGGCACTGTCGTTGTTGC |
| | | CGACACTGGTGATTTCGGCTCTATTGCCAAGTTTCAACCTCA |
| | | AGACTCCACAACTAACCCATCATTGATCTTGGCTGCTGCCAA |
| | | GCAACCAACTTACGCCAAGTTGATCGATGTTGCCGTGGAAT |
| | | ACGGTAAGAAGCATGGTAAGACCACCGAAGAACAAGTCGA |
| | | AAATGCTGTGGACAGATTGTTAGTCGAATTCGGTAAGGAGA |
| | | TCTTAAAGATTGTTCCAGGCAGAGTCTCCACCGAAGTTGAT |
| | | GCTAGATTGTCTTTTGACACTCAAGCTACCATTGAAAAGGCT |
| | | AGACATATCATTAAATTGTTTGAACAAGAAGGTGTCTCCAA |
| | | GGAAAGAGTCCTTATTAAAATTGCTTCCACTTGGGAAGGTA |
| | | TTCAAGCTGCCAAAGAATTGGAAGAAAAGGACGGTATCCAC |
| | | TGTAATTTGACTCTATTATTCTCCTTCGTTCAAGCAGTTGCCT |
| | | GTGCCGAGGCCCAAGTTACTTTGATTTCCCCATTTGTTGGTA |
| | | GAATTCTAGACTGGTACAAATCCAGCACTGGTAAAGATTAC |
| | | AAGGGTGAAGCCGACCCAGGTGTTATTTCCGTCAAGAAAAT |
| | | CTACAACTACTACAAGAAGTACGGTTACAAGACTATTGTTA |
| | | TGGGTGCTTCTTTCAGAAGCACTGACGAAATCAAAAACTTG |
| | | GCTGGTGTTGACTATCTAACAATTTCTCCAGCTTTATTGGAC |
| | | AAGTTGATGAACAGTACTGAACCTTTCCCAAGAGTTTTGGA |
| | | CCCTGTCTCCGCTAAGAAGGAAGCCGGCGACAAGATTTCTT |
| | | ACATCAGCGACGAATCTAAATTCAGATTCGACTTGAATGAA |
| | | GACGCTATGGCCACTGAAAAATTGTCCGAAGGTATCAGAAA |
| | | ATTCTCTGCCGATATTGTTACTCTATTCGACTTGATTGAAAA |
| | | GAAAGTTACCGCTTAA |
| | | (SEQ ID NO: 86) |
| Ribulose- | 5.1.3.1 | >RPE1 YJL121C Chr 10 |
| phosphate 3- | | ATGGTCAAACCAATTATAGCTCCCAGTATCCTTGCTTCTGAC |
| epimerase | | TTCGCCAACTTGGGTTGCGAATGTCATAAGGTCATCAACGC |
| | | CGGCGCAGATTGGTTACATATCGATGTCATGGACGGCCATT |
| | | TTGTTCCAAACATTACTCTGGGCCAACCAATTGTTACCTCCC |
| | | TACGTCGTTCTGTGCCACGCCCTGGCGATGCTAGCAACACA |
| | | GAAAAGAAGCCCACTGCGTTCTTCGATTGTCACATGATGGT |
| | | TGAAAATCCTGAAAAATGGGTCGACGATTTTGCTAAATGTG |
| | | GTGCTGACCAATTTACGTTCCACTACGAGGCCACACAAGAC |
| | | CCTTTGCATTTAGTTAAGTTGATTAAGTCTAAGGGCATCAAA |
| | | GCTGCATGCGCCATCAAACCTGGTACTTCTGTTGACGTTTTA |
| | | TTTGAACTAGCTCCTCATTTGGATATGGCTCTTGTTATGACT |
| | | GTGGAACCTGGGTTTGGAGGCCAAAAATTCATGGAAGACAT |
| | | GATGCCAAAAGTGGAAACTTTGAGAGCCAAGTTCCCCCATT |
| | | TGAATATCCAAGTCGATGGTGGTTTGGGCAAGGAGACCATC |
| | | CCGAAAGCCGCCAAAGCCGGTGCCAACGTTATTGTCGCTGG |
| | | TACCAGTGTTTTCACTGCAGCTGACCCGCACGATGTTATCTC |
| | | CTTCATGAAAGAAGAAGTCTCGAAGGAATTGCGTTCTAGAG |
| | | ATTTGCTAGATTAG |
| | | (SEQ ID NO: 87) |
| Ribose-5- | 5.3.1.6 | >RKI1 YOR095C Chr 15 |
| phosphate | | ATGGCTGCCGGTGTCCCAAAAATTGATGCGTTAGAATCTTTG |
| isomerase | | GGCAATCCTTTGGAGGATGCCAAGAGAGCTGCAGCATACAG |
| | | AGCAGTTGATGAAAATTTAAAATTTGATGATCACAAAATTA |
| | | TTGGAATTGGTAGTGGTAGCACAGTGGTTTATGTTGCCGAA |
| | | AGAATTGGACAATATTTGCATGACCCTAAATTTTATGAAGT |
| | | AGCGTCTAAATTCATTTGCATTCCAACAGGATTCCAATCAAG |
| | | AAACTTGATTTTGGATAACAAGTTGCAATTAGGCTCCATTGA |
| | | ACAGTATCCTCGCATTGATATAGCGTTTGACGGTGCTGATGA |
| | | AGTGGATGAGAATTTACAATTAATTAAAGGTGGTGGTGCTT |
| | | GTCTATTTCAAGAAAAATTGGTTAGTACTAGTGCTAAAACCT |
| | | TCATTGTCGTTGCTGATTCAAGAAAAAAGTCACCAAAACAT |
| | | TTAGGTAAGAACTGGAGGCAAGGTGTTCCCATTGAAATTGT |
| | | ACCTTCCTCATACGTGAGGGTCAAGAATGATCTATTAGAAC |
| | | AATTGCATGCTGAAAAAGTTGACATCAGACAAGGAGGTTCT |
| | | GCTAAAGCAGGTCCTGTTGTAACTGACAATAATAACTTCATT |
| | | ATCGATGCGGATTTCGGTGAAATTTCCGATCCAAGAAAATT |
| | | GCATAGAGAAATCAAACTGTTAGTGGGCGTGGTGGAAACAG |
| | | GTTTATTCATCGACAACGCTTCAAAAGCCTACTTCGGTAATT |
| | | CTGACGGTAGTGTTGAAGTTACCGAAAAGTGA |
| | | (SEQ ID NO: 88) |

In addition, the microorganism can comprise any combination of polynucleotides encoding polypeptides that function in the pentose phosphate pathway.

In some embodiments, the compositions used herein comprise both microorganisms capable of producing butanol and microorganisms that are not capable of producing butanol. Lignocellulosic hydrolysates can inhibit the growth of butanol-producing microorganisms and can do so to a greater extent than they inhibit the growth of non-butanol-producing microorganisms. The methods described herein, maximize the growth and yield of butanol-producing microorganisms.

In some embodiments, the butanol-producing microorganisms are present in a composition (e.g., a fermenting composition) at a concentration that is at least equal to the concentration of microorganisms that are not capable of producing butanol. In addition, the microorganisms capable of producing butanol can be present at a concentration that is greater than the concentration of microorganisms that are not capable of producing butanol. The microorganisms capable of producing butanol can be present at a concentration that is at least twice the concentration of microorganisms that are not capable of producing butanol.

Methods for Isobutanol Isolation from the Fermentation Medium

According to the methods described herein, butanol can be obtained from 5-carbon sugars by a method comprising (a) providing a composition comprising a microorganism capable of producing butanol and an enzyme or enzymes capable of converting a 5-carbon sugar to xylulose; (b) contacting the composition with a source of 5-carbon sugars; (c) culturing the microorganism under conditions that limits respiration; and (d) purifying isobutanol from the culture.

Methods described herein can be used in conjunction with methods known in the art. Methods that can be used in conjunction with methods disclosed herein are disclosed in U.S. Provisional Application No. 61/356,290, filed on Jun. 18, 2010; as well as U.S. Provisional Application No. 61/368,451, filed on Jul. 28, 2010; U.S. Provisional Application No. 61/368,436, filed on Jul. 28, 2010; U.S. Provisional Application No. 61/368,444, filed on Jul. 28, 2010; U.S. Provisional Application No. 61/368,429, filed on Jul. 28, 2010; U.S. Provisional Application No. 61/379,546, filed on Sep. 2, 2010; and U.S. Provisional Application No. 61/440,034, filed on Feb. 7, 2011; the entire contents of which are all herein incorporated by reference.

Bioproduced isobutanol can be isolated from the fermentation medium using methods known in the art for acetone-butanol-ethanol (ABE) fermentations (see, e.g., Durre, Appl. Microbiol. Biotechnol. 49:639-648 (1998), Groot et al., Process. Biochem. 27:61-75 (1992), and references therein). For example, solids can be removed from the fermentation medium by centrifugation, filtration, decantation, or the like. Then, the isobutanol can be isolated from the fermentation medium using methods such as distillation, azeotropic distillation, liquid-liquid extraction, adsorption, gas stripping, membrane evaporation, or pervaporation.

Because isobutanol forms a low boiling point, azeotropic mixture with water, distillation can be used to separate the mixture up to its azeotropic composition. Distillation can be used in combination with another separation method to obtain separation around the azeotrope. Methods that can be used in combination with distillation to isolate and purify butanol include, but are not limited to, decantation, liquid-liquid extraction, adsorption, and membrane-based techniques. Additionally, butanol can be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation can be used in combination with decantation to isolate and purify the isobutanol. In this method, the isobutanol containing fermentation broth is distilled to near the azeotropic composition. Then, the azeotropic mixture is condensed, and the isobutanol is separated from the fermentation medium by decantation. The decanted aqueous phase can be returned to the first distillation column as reflux. The isobutanol-rich decanted organic phase can be further purified by distillation in a second distillation column.

The isobutanol can also be isolated from the fermentation medium using liquid-liquid extraction in combination with distillation. In this method, the isobutanol is extracted from the fermentation broth using liquid-liquid extraction with a suitable solvent. The isobutanol-containing organic phase is then distilled to separate the butanol from the solvent.

Distillation in combination with adsorption can also be used to isolate isobutanol from the fermentation medium. In this method, the fermentation broth containing the isobutanol is distilled to near the azeotropic composition and then the remaining water is removed by use of an adsorbent, such as molecular sieves (Aden et al. Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation can be used to isolate and purify the isobutanol from the fermentation medium. In this method, the fermentation broth containing the isobutanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, the alcohol can be esterified by contacting the alcohol in a fermentation medium with an organic acid (e.g., fatty acids) and a catalyst (e.g. lipase) capable of esterifying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

### EXAMPLES

The present invention is further defined in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

### Example 1

### Conversion of Fermentable Carbons in Lignocellulosic Hydrolysates to Isobutanol Methods

Lignocellulosic hydrolysate (LCH) was produced from ground corn cob that had been pretreated by a dilute ammonia and heat process then enzymatically hydrolyzed with a mixture of commercial cellulase and hemicellulase enzyme preparations at 25% percent pretreated corn cob solids, pH 5.3 and 48° C. for 96 hours, all as described in U.S. Publication No. 2007/0031918A1, which is herein incorporated by reference. The primary sugar and acetate concentrations in the resulting hydrolysate were: 75 g/L glucose; 54 g/L xylose, 6 g/L arabinose, and 5 g/L acetate.

Two yeast strains were used. The first, CEN.PKI 13-7D, is a wildtype ethanologenic strain. Van Dijken et al., Enzyme Microb Technol 26:706-714 (2000). The second strain, PNY1504 is an isobutanologenic strain. The strain was created from PNY1503 (MATa ura3Δ::loxP his3Δ pdc6Δ pdc1Δ::P[PDC1]-DHADIilvD_Sm-PDClt pdc5Δ::P[PDC5]-ADH|sadB_Ax-PDC5t gpd2Δ::loxP) by transformation with plasmids pYZ090 (alsS-L. lactis KAR1) and pLH468 (IlvD-hADH-KivDy).

Synthetic Complete-GE medium consisted of Yeast Nitrogen Base w/o amino acids, dropout mix-His-Ura-Trp-Leu (1.4 g/L, Sigma Y2001) plus tryptophan (20 mg/L) and leucine (60 mg/L) (Sherman F, Methods in Enzymology 350:3-41 (2002)), and 3 g/L glucose plus 3 ml/L 190 proof ethanol (Sigma E7023). Liquid medium was buffered to pH 5.5 with 0.1 M MES-KOH. Solid medium for Petri plates was formed with 20 g agar/L.

Test tubes containing 2 ml of SC-GE medium were inoculated from a plate, and incubated at 30° C. with shaking for 6 hours. Then this pre-culture was used to inoculate 50 ml SC-GE in a 250 ml flask for overnight incubation at 30° C., 250 rpm. Cells were recovered by centrifugation and transferred to 0.10 ml of production medium in 50 ml flasks. Cultures were propagated at 30° C. for 150 hours and sampled periodically for analysis of residual sugar and produced alcohol by HPLC. The production media tested were either LCH or LCH diluted 1:1 with water.

Before analysis, fermentation samples were passed through a Nanosep MF 0.2 micron centrifugal filter (Pall Life Sciences, Ann Arbor, Mich.) using a Microfuge 18 Centrifuge (Beckman Coulter) set at 13,000 rpm for 3-5 minute. Glucose, xylose, acetic acid, glycerol, ethanol, and isobutanol in the fermentation broth were measured by HPLC with a Waters Alliance HPLC system. The column used was a Transgenomic ION-300 column (#ICE-99-9850, Transgenomic, Inc., Omaha, Nebr.) with a BioRad Micro-Guard Cartridge Cation-H (#125-0129, Bio-Rad, Hercules, Calif.). The column was run at 75° C. and 0.4 mL/min flow rate using 0.01 N H₂SO₄ as solvent. The concentrations of starting sugars and products were determined with a refractive index detector using external standard calibration curves.

### Results

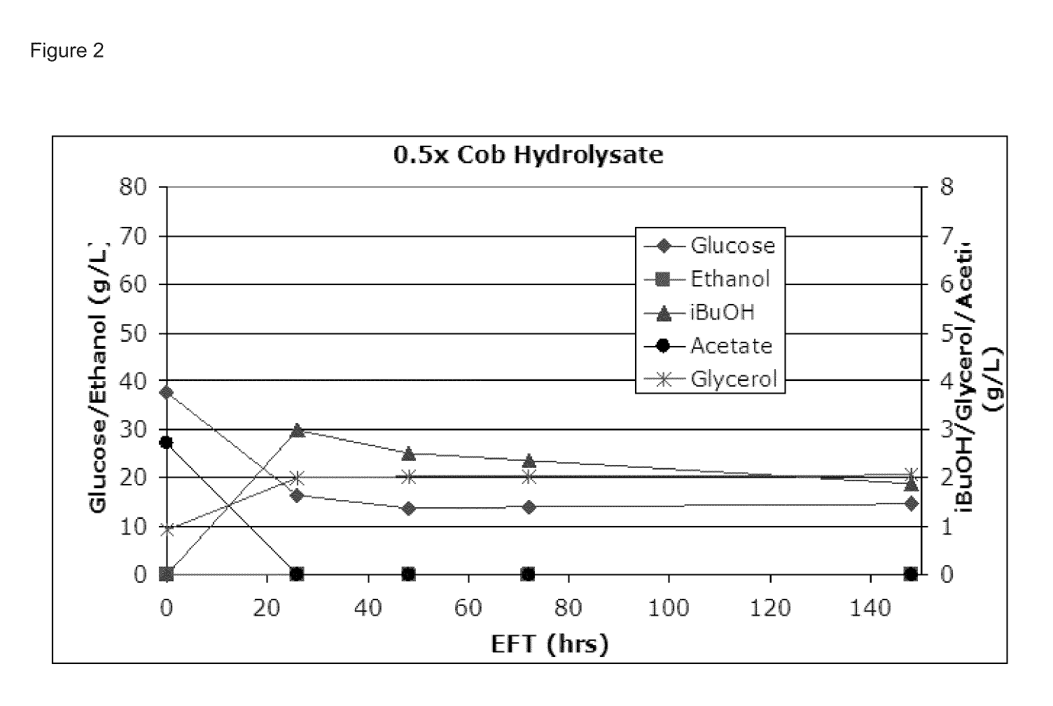

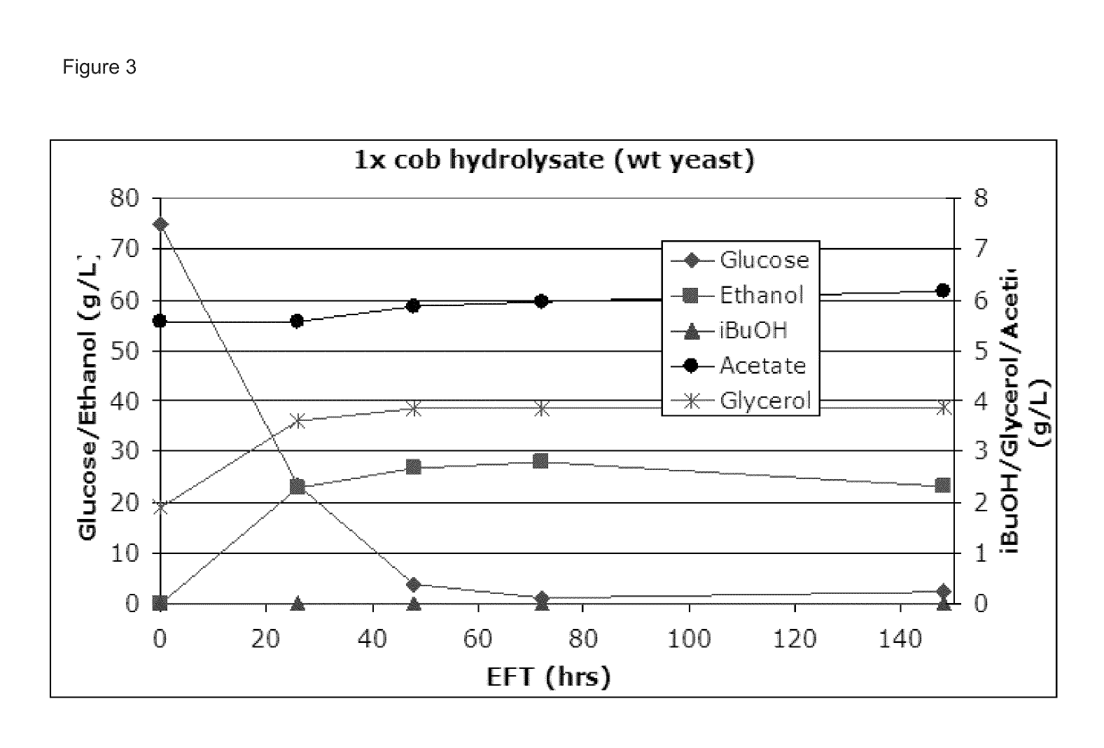

The isobutanologen, PNY1504, was unable to grow on 1× corn cob hydrolysate. As can be seen in FIG. 1, it grew on diluted hydrolysate at a rate comparable to the wildtype strain on undiluted LCH, and it achieved approximately ⅔ the final biomass concentration of the wildtype strain. FIG. 2 shows the profiles of glucose consumption and isobutanol production by PNY1504. Glucose was consumed from −40 g/L down to a residual concentration of −15 g/L within 24 hours. In that same period, isobutanol was produced with a final titer of 3 g/L, resulting in a yield of 0.12 g·g⁻¹. By comparison, in FIG. 3, the ethanologenic strain is observed to consume the glucose almost completely, from an initial concentration of −75 g/L down to <5 g/L, over a period of >48 hours. It produced approximately 28 g/L ethanol, for a yield of −0.37 g·g⁻¹.

### Example 2

### Conversion of C-5 Sugars to C-4 Alcohol in Defined Medium

### Methods

Strain PNY1504 was pre-cultured in the defined medium SC-GE as described above, except that the medium was buffered to pH 6. Production cultures used the same SC medium, except either glucose or xylose was added to a final concentration of 35 g/L, and penicillin G (Sigma P3032) was added at 25 μg/ml.

Unless it is genetically engineered to do so, S. cerevisiae is unable to ferment xylose, but it is able to ferment xylulose. In order to test whether xylose is available for fermentation to isobutanol, it was converted to xylulose in situ by xylose isomerase (10 g/L; Sigma G4166) essentially as described in Lastick S. M., et al., Applied Microbiology and Biotechnology 30:574-579 (1989), Wang P. Y., et al., Biotechnology Letters 2:273-278 (1980) and Chandrakant P & Bisaria V S, Appl Microbiol Biotechnol 53:301-309 (2000).

Xylulose can be taken up by yeast and metabolized via the pentose phosphate pathway. It has been shown that yeast displays a predominantly respiratory mode of metabolism when grown on xylose, which results in a high biomass yield and low yields of fermentative products. Souto-Maior A M, et al., J Biotechnol. 143:119-23 (2009). Thus, in order to increase flux towards fermentative products, cultures were treated with the respiratory inhibitor antimycin A (1 μM; Sigma A8674).

### Results

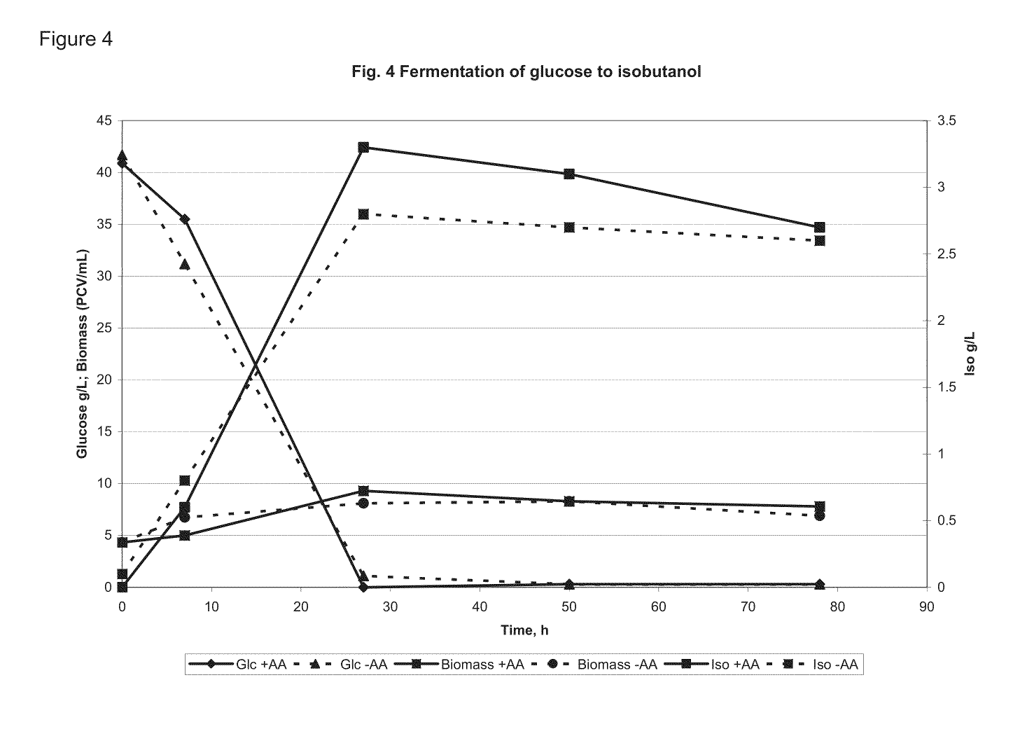

As shown in FIG. 4, the isobutanologen fermented glucose to isobutanol. The hexose was consumed within 24 hours, irrespective of antimycin A treatment. The antimycin A-treated culture achieved a somewhat higher isobutanol titer, ˜3.3 g/L, for a yield of approximately 0.08 g·g⁻¹. Low levels of growth were observed, since a high initial biomass concentration was used to inoculate the production cultures.

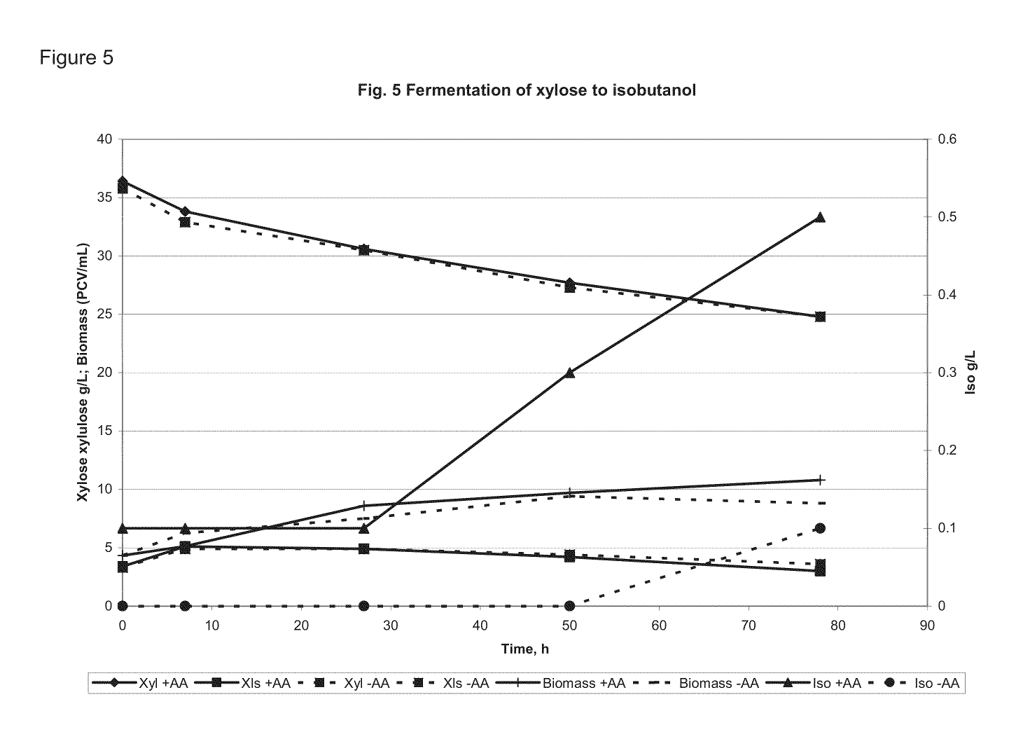

FIG. 5 shows the fermentation profile when xylose was provided as the sole carbon source, along with xylose isomerase. Xylose conversion to xylulose and its subsequent utilization are slower than glucose consumption. By 78 hours, only 10 g/L of xylose had been consumed (indirectly, as xylulose, with which it is in equilibrium due to the presence of xylose isomerase). This consumption profile was not significantly affected by the presence or absence of antimycin A. However, the respiratory inhibitor did reduce biomass accumulation slightly, and a significantly increased production of isobutanol was observed (0.5 vs. 0.1 g/L at 78 hours, respectively). The isobutanol yield was 0.04 g·g⁻¹ in the presence of antimycin A and 0.01 g·g⁻¹ in the absence of the drug.

### Example 3

### Conversion of Xylose in Lignocellulosic Hydrolysates to Isobutanol

### Methods

This experiment used xylose isomerase to convert xylose present in lignocellulosic hydrolysate (LCH) to xylulose, which is then available for fermentation to isobutanol by isobutanologenic yeast strains. PNY1504 was pre-grown as described above and transferred into 0.5×LCH containing penicillin G at 25 mg/L for cultivation. Xylose isomerase (10 g/L) and/or antimycin A (1 μM) were added as described in the Figure legends. Samples were withdrawn periodically for analysis during the course of 170 hours.

### Results

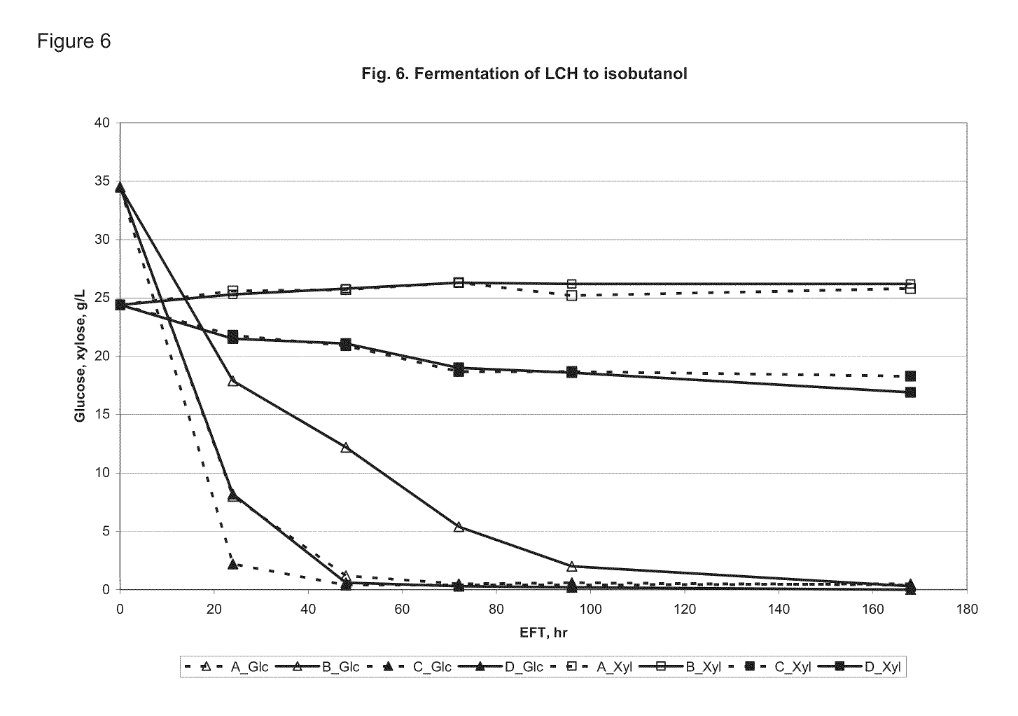

FIG. 6 shows the concentrations of glucose, xylose, and xylulose during the fermentation. Glucose was consumed within 48 hours, except when antimycin A was added in the absence of xylose isomerase. Xylose consumption and the formation of xylulose (not shown) required the addition of xylose isomerase, as expected.

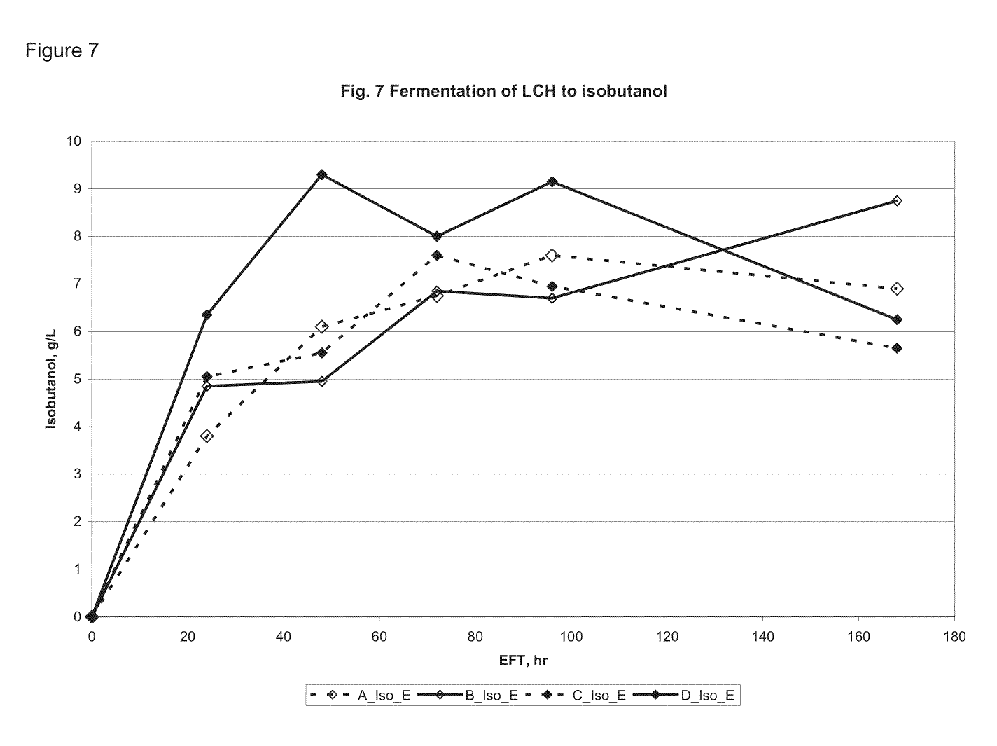

The effective isobutanol titer during the fermentation is shown in FIG. 7. All four cultures made isobutanol from glucose during the first 48 hours, with titers ranging from approximately 4-6 g/L. Subsequently, in the period after glucose was exhausted from the feedstock, the culture treated with both xylose isomerase and antimycin A made the highest amount of isobutanol, through 100 hours. The concentration subsequently declined, presumably due to evaporation of the alcohol. The culture without xylose isomerase continued to gradually accumulate isobutanol throughout the experiment, possibly due to the gradual assimilation of poor carbon sources in the hydrolysate such as acetic acid.

### Example 3

### Recovery of Isobutanol

The isobutanol produced in the preceding Examples may be recovered by in situ product recovery process in accordance with the methods of U.S. Provisional Application No. 61/356,290, filed on Jun. 18, 2010. The in situ product recovery (ISPR) methods described therein provide for improved butanol production by the removal of inhibitors prior to and during fermentation. The utilization of mixed sugars by the recombinant organism with the ISPR techniques may provide for improvements in butanol production through one or more if increased sugar utilization, decreased inhibitor profiles and increased alcohol product tolerance.

### Example 4

### Construction of Saccharomyces cerevisiae Strain BP1083 (“NGC1-070”; PNY 1504)

The strain BP1064 was derived from CEN.PK 113-7D (CBS 8340; Centraalbureau voor Schimmelcultures (CBS) Fungal Biodiversity Centre, Netherlands) and contains deletions of the following genes: URA3, HIS3, PDC1, PDC5, PDC6, and GPD2. BP1064 was transformed with plasmids pYZ090 (SEQ ID NO: 1, described in U.S. Provisional Application Ser. No. 61/246,844) and pLH468 (SEQ ID NO: 2) to create strain NGC1-070 (BP1083, PNY1504).

Deletions, which completely removed the entire coding sequence, were created by homologous recombination with PCR fragments containing regions of homology upstream and downstream of the target gene and either a G418 resistance marker or URA3 gene for selection of transformants. The G418 resistance marker, flanked by loxP sites, was removed using Cre recombinase. The URA3 gene was removed by homologous recombination to create a scarless deletion or if flanked by loxP sites, was removed using Cre recombinase.

The scarless deletion procedure was adapted from Akada, et al., (Yeast 23:399-405, 2006). In general, the PCR cassette for each scarless deletion was made by combining four fragments, A-B-U-C, by overlapping PCR. The PCR cassette contained a selectable/counter-selectable marker, URA3 (Fragment U), consisting of the native CEN.PK 113-7D URA3 gene, along with the promoter (250 bp upstream of the URA3 gene) and terminator (150 bp downstream of the URA3 gene). Fragments A and C, each 500 bp long, corresponded to the 500 bp immediately upstream of the target gene (Fragment A) and the 3′ 500 bp of the target gene (Fragment C). Fragments A and C were used for integration of the cassette into the chromosome by homologous recombination. Fragment B (500 bp long) corresponded to the 500 bp immediately downstream of the target gene and was used for excision of the URA3 marker and Fragment C from the chromosome by homologous recombination, as a direct repeat of the sequence corresponding to Fragment B was created upon integration of the cassette into the chromosome. Using the PCR product ABUC cassette, the URA3 marker was first integrated into and then excised from the chromosome by homologous recombination. The initial integration deleted the gene, excluding the 3′ 500 bp. Upon excision, the 3′ 500 bp region of the gene was also deleted. For integration of genes using this method, the gene to be integrated was included in the PCR cassette between fragments A and B.

### URA3 Deletion

To delete the endogenous URA3 coding region, a ura3::loxP-kanMX-loxP cassette was PCR-amplified from pLA54 template DNA (SEQ ID NO: 3). pLA54 contains the K. lactis TEFI promoter and kanMX marker, and is flanked by loxP sites to allow recombination with Cre recombinase and removal of the marker. PCR was done using Phusion® DNA polymerase (New England BioLabs Inc., Ipswich, Mass.) and primers BK505 and BK506 (SEQ ID NOs: 4 and 5). The URA3 portion of each primer was derived from the 5′ region upstream of the URA3 promoter and 3′ region downstream of the coding region such that integration of the loxP-kanMX-loxP marker resulted in replacement of the URA3 coding region. The PCR product was transformed into CEN.PK 113-7D using standard genetic techniques (Methods in Yeast Genetics, 2005, Cold Spring Harbor Laboratory Press, Cold Spring Harbor, N.Y., pp. 201-202) and transformants were selected on YPD containing G418 (100 μg/mL) at 30° C. Transformants were screened to verify correct integration by PCR using primers LA468 and LA492 (SEQ ID NOs: 6 and 7) and designated CEN.PK 113-7ΔΔura3::kanMX.

### HIS3 Deletion

The four fragments for the PCR cassette for the scarless HIS3 deletion were amplified using Phusion® High Fidelity PCR Master Mix (New England BioLabs Inc., Ipswich, Mass.) and CEN.PK 113-7D genomic DNA as template, prepared with a Gentra® Puregene® Yeast/Bact, kit (Qiagen, Valencia, Calif.). HIS3 Fragment A was amplified with primer oBP452 (SEQ ID NO: 14) and primer oBP453 (SEQ ID NO: 15) containing a 5′ tail with homology to the 5′ end of HIS3 Fragment B. HIS3 Fragment B was amplified with primer oBP454 (SEQ ID NO: 16) containing a 5′ tail with homology to the 3′ end of HIS3 Fragment A, and primer oBP455 (SEQ ID NO: 17) containing a 5′ tail with homology to the 5′ end of HIS3 Fragment U. HIS3 Fragment U was amplified with primer oBP456 (SEQ ID NO: 18) containing a 5′ tail with homology to the 3′ end of HIS3 Fragment B, and primer oBP457 (SEQ ID NO: 19) containing a 5′ tail with homology to the 5′ end of HIS3 Fragment C. HIS3 Fragment C was amplified with primer oBP458 (SEQ ID NO: 20) containing a 5′ tail with homology to the 3′ end of HIS3 Fragment U, and primer oBP459 (SEQ ID NO: 21). PCR products were purified with a PCR Purification kit (Qiagen, Valencia, Calif.). HIS3 Fragment AB was created by overlapping PCR by mixing HIS3 Fragment A and HIS3 Fragment B and amplifying with primers oBP452 (SEQ ID NO: 14) and oBP455 (SEQ ID NO: 17). HIS3 Fragment UC was created by overlapping PCR by mixing HIS3 Fragment U and HIS3 Fragment C and amplifying with primers oBP456 (SEQ ID NO: 18) and oBP459 (SEQ ID NO: 21). The resulting PCR products were purified on an agarose gel followed by a Gel Extraction kit (Qiagen, Valencia, Calif.). The HIS3 ABUC cassette was created by overlapping PCR by mixing HIS3 Fragment AB and HIS3 Fragment UC and amplifying with primers oBP452 (SEQ ID NO: 14) and oBP459 (SEQ ID NO: 21). The PCR product was purified with a PCR Purification kit (Qiagen, Valencia, Calif.).

Competent cells of CEN.PK 113-7D Δura3::kanMX were made and transformed with the HIS3 ABUC PCR cassette using a Frozen-EZ Yeast Transformation II™ kit (Zymo Research Corporation, Irvine, Calif.). Transformation mixtures were plated on synthetic complete media lacking uracil supplemented with 2% glucose at 30° C. Transformants with a his3 knockout were screened for by PCR with primers oBP460 (SEQ ID NO: 22) and oBP461 (SEQ ID NO: 23) using genomic DNA prepared with a Gentra® Puregene® Yeast/Bact. kit (Qiagen, Valencia, Calif.). A correct transformant was selected as strain CEN.PK 113-7D Δura3::kanMX Δhis3::URA3.

KanMX Marker Removal from the Δura3 Site and URA3 μMarker Removal from the Δhis3 Site

The KanMX marker was removed by transforming CEN.PK 113-7D Δura3::kanMX Δhis3::URA3 with pRS423::PGAL1-cre (SEQ ID NO: 66, described in U.S. Provisional Application No. 61/290,639) using a Frozen-EZ Yeast Transformation II™ kit (Zymo Research Corporation, Irvine, Calif.) and plating on synthetic complete medium lacking histidine and uracil supplemented with 2% glucose at 30° C. Transformants were grown in YP supplemented with 1% galactose at 30° C. for ˜6 hours to induce the Cre recombinase and KanMX marker excision and plated onto YPD (2% glucose) plates at 30° C. for recovery. An isolate was grown overnight in YPD and plated on synthetic complete medium containing 5-fluoro-orotic acid (5-FOA, 0.1%) at 30° C. to select for isolates that lost the URA3 marker. 5-FOA resistant isolates were grown in and plated on YPD for removal of the pRS423::PGAL1-cre plasmid. Isolates were checked for loss of the KanMX marker, URA3 marker, and pRS423::PGAL1-cre plasmid by assaying growth on YPD+G418 plates, synthetic complete medium lacking uracil plates, and synthetic complete medium lacking histidine plates. A correct isolate that was sensitive to G418 and auxotrophic for uracil and histidine was selected as strain CEN.PK 113-7D Δura3::loxP Δhis3 and designated as BP857. The deletions and marker removal were confirmed by PCR and sequencing with primers oBP450 (SEQ ID NO: 24) and oBP451 (SEQ ID NO: 25) for Δura3 and primers oBP460 (SEQ ID NO: 22) and oBP461 (SEQ ID NO: 23) for Δhis3 using genomic DNA prepared with a Gentra® Puregene® Yeast/Bact. kit (Qiagen, Valencia, Calif.).

### PDC6 Deletion

The four fragments for the PCR cassette for the scarless PDC6 deletion were amplified using Phusion® High Fidelity PCR Master Mix (New England BioLabs Inc., Ipswich, Mass.) and CEN.PK 113-7D genomic DNA as template, prepared with a Gentra® Puregene® Yeast/Bact. kit (Qiagen, Valencia, Calif.). PDC6 Fragment A was amplified with primer oBP440 (SEQ ID NO: 26) and primer oBP441 (SEQ ID NO: 27) containing a 5′ tail with homology to the 5′ end of PDC6 Fragment B. PDC6 Fragment B was amplified with primer oBP442 (SEQ ID NO: 28), containing a 5′ tail with homology to the 3′ end of PDC6 Fragment A, and primer oBP443 (SEQ ID NO: 29) containing a 5′ tail with homology to the 5′ end of PDC6 Fragment U. PDC6 Fragment U was amplified with primer oBP444 (SEQ ID NO: 30) containing a 5′ tail with homology to the 3′ end of PDC6 Fragment B, and primer oBP445 (SEQ ID NO: 31) containing a 5′ tail with homology to the 5′ end of PDC6 Fragment C. PDC6 Fragment C was amplified with primer oBP446 (SEQ ID NO: 32) containing a 5′ tail with homology to the 3′ end of PDC6 Fragment U, and primer oBP447 (SEQ ID NO: 33). PCR products were purified with a PCR Purification kit (Qiagen, Valencia, Calif.). PDC6 Fragment AB was created by overlapping PCR by mixing PDC6 Fragment A and PDC6 Fragment B and amplifying with primers oBP440 (SEQ ID NO: 26) and oBP443 (SEQ ID NO: 29). PDC6 Fragment UC was created by overlapping PCR by mixing PDC6 Fragment U and PDC6 Fragment C and amplifying with primers oBP444 (SEQ ID NO: 30) and oBP447 (SEQ ID NO: 33). The resulting PCR products were purified on an agarose gel followed by a Gel Extraction kit (Qiagen, Valencia, Calif.). The PDC6 ABUC cassette was created by overlapping PCR by mixing PDC6 Fragment AB and PDC6 Fragment UC and amplifying with primers oBP440 (SEQ ID NO: 26) and oBP447 (SEQ ID NO: 33). The PCR product was purified with a PCR Purification kit (Qiagen, Valencia, Calif.).

Competent cells of CEN.PK 113-7D Δura3::loxP Δhis3 were made and transformed with the PDC6 ABUC PCR cassette using a Frozen-EZ Yeast Transformation II™ kit (Zymo Research Corporation, Irvine, Calif.). Transformation mixtures were plated on synthetic complete media lacking uracil supplemented with 2% glucose at 30° C. Transformants with a pdc6 knockout were screened for by PCR with primers oBP448 (SEQ ID NO: 34) and oBP449 (SEQ ID NO: 35) using genomic DNA prepared with a Gentra® Puregene® Yeast/Bact. kit (Qiagen, Valencia, Calif.). A correct transformant was selected as strain CEN.PK 113-7D Δura3::loxP Δhis3 Δpdc6::URA3.

CEN.PK 113-7D Δura3::loxP Δhis3 Δpdc6::URA3 was grown overnight in YPD and plated on synthetic complete medium containing 5-fluoro-orotic acid (0.1%) at 30° C. to select for isolates that lost the URA3 marker. The deletion and marker removal were confirmed by PCR and sequencing with primers oBP448 (SEQ ID NO: 34) and oBP449 (SEQ ID NO: 35) using genomic DNA prepared with a Gentra® Puregene® Yeast/Bact. kit (Qiagen, Valencia, Calif.). The absence of the PDC6 gene from the isolate was demonstrated by a negative PCR result using primers specific for the coding sequence of PDC6, oBP554 (SEQ ID NO: 36) and oBP555 (SEQ ID NO: 37). The correct isolate was selected as strain CEN.PK 113-7D Δura3::loxP Δhis3 Δpdc6 and designated as BP891.

PDC1 Deletion ilvDSm Integration

The PDC1 gene was deleted and replaced with the ilvD coding region from Streptococcus mutans ATCC No. 700610. The A fragment followed by the ilvD coding region from Streptococcus mutans for the PCR cassette for the PDC1 deletion-ilvDSm integration was amplified using Phusion® High Fidelity PCR Master Mix (New England BioLabs Inc., Ipswich, Mass.) and NYLA83 genomic DNA as template, prepared with a Gentra® Puregene® Yeast/Bact. kit (Qiagen, Valencia, Calif.). NYLA83 is a strain (construction described in U.S. Patent Application Publication No. 2011/0124060, incorporated herein by reference in its entirety) which carries the PDC1 deletion-ilvDSm integration described in U.S. Patent Application Publication No. 2009/0305363, herein incorporated by reference in its entirety). PDC1 Fragment A-ilvDSm (SEQ ID NO: 69) was amplified with primer oBP513 (SEQ ID NO: 38) and primer oBP515 (SEQ ID NO: 39) containing a 5′ tail with homology to the 5′ end of PDC1 Fragment B. The B, U, and C fragments for the PCR cassette for the PDC1 deletion-ilvDSm integration were amplified using Phusion® High Fidelity PCR Master Mix (New England BioLabs Inc., Ipswich, Mass.) and CEN.PK 113-7D genomic DNA as template, prepared with a Gentra® Puregene® Yeast/Bact. kit (Qiagen, Valencia, Calif.). PDC1 Fragment B was amplified with primer oBP516 (SEQ ID NO: 40) containing a 5′ tail with homology to the 3′ end of PDC1 Fragment A-ilvDSm, and primer oBP517 (SEQ ID NO: 41) containing a 5′ tail with homology to the 5′ end of PDC1 Fragment U. PDC1 Fragment U was amplified with primer oBP518 (SEQ ID NO: 42) containing a 5′ tail with homology to the 3′ end of PDC1 Fragment B, and primer oBP519 (SEQ ID NO: 43) containing a 5′ tail with homology to the 5′ end of PDC1 Fragment C. PDC1 Fragment C was amplified with primer oBP520 (SEQ ID NO: 44), containing a 5′ tail with homology to the 3′ end of PDC1 Fragment U, and primer oBP521 (SEQ ID NO: 45). PCR products were purified with a PCR Purification kit (Qiagen, Valencia, Calif. PDC1 Fragment A-ilvDSm-B was created by overlapping PCR by mixing PDC1 Fragment A-ilvDSm and PDC1 Fragment B and amplifying with primers oBP513 (SEQ ID NO: 38) and oBP517 (SEQ ID NO: 41). PDC1 Fragment UC was created by overlapping PCR by mixing PDC1 Fragment U and PDC1 Fragment C and amplifying with primers oBP518 (SEQ ID NO: 42) and oBP521 (SEQ ID NO: 45). The resulting PCR products were purified on an agarose gel followed by a Gel Extraction kit (Qiagen, Valencia, Calif.). The PDC1 A-ilvDSm-BUC cassette (SEQ ID NO: 70) was created by overlapping PCR by mixing PDC1 Fragment A-ilvDSm-B and PDC1 Fragment UC and amplifying with primers oBP513 (SEQ ID NO: 38) and oBP521 (SEQ ID NO: 45). The PCR product was purified with a PCR Purification kit (Qiagen, Valencia, Calif.).

Competent cells of CEN.PK 113-7D Δura3::loxP Δhis3 Δpdc6 were made and transformed with the PDC1 A-ilvDSm-BUC PCR cassette using a Frozen-EZ Yeast Transformation II™ kit (Zymo Research Corporation, Irvine, Calif.). Transformation mixtures were plated on synthetic complete media lacking uracil supplemented with 2% glucose at 30° C. Transformants with a pdc1 knockout ilvDSm integration were screened for by PCR with primers oBP511 (SEQ ID NO: 46) and oBP512 (SEQ ID NO: 47) using genomic DNA prepared with a Gentra® Puregene® Yeast/Bact. kit (Qiagen, Valencia, Calif.). The absence of the PDC1 gene from the isolate was demonstrated by a negative PCR result using primers specific for the coding sequence of PDC1, oBP550 (SEQ ID NO: 48) and oBP551 (SEQ ID NO: 49). A correct transformant was selected as strain CEN.PK 113-7D Δura3::loxP Δhis3 Δpdc6 Δpdc1::ilvDSm-URA3.

CEN.PK 113-7D Δura3::loxP Δhis3 Δpdc6 Δpdc1::ilvDSm-URA3 was grown overnight in YPD and plated on synthetic complete medium containing 5-fluoro-orotic acid (0.1%) at 30° C. to select for isolates that lost the URA3 marker. The deletion of PDC1, integration of ilvDSm, and marker removal were confirmed by PCR and sequencing with primers oBP511 (SEQ ID NO: 46) and oBP512 (SEQ ID NO: 47) using genomic DNA prepared with a Gentra® Puregene® Yeast/Bact. kit (Qiagen, Valencia, Calif.). The correct isolate was selected as strain CEN.PK 113-7D Δura3::loxP Δhis3 Δpdc6 Δpdc1::ilvDSm and designated as BP907.

PDC5 Deletion sadB Integration

The PDC5 gene was deleted and replaced with the sadB coding region from Achromobacter xylosoxidans. A segment of the PCR cassette for the PDC5 deletion-sadB integration was first cloned into plasmid pUC19-URA3MCS.

pUC19-URA3MCS is pUC19 based and contains the sequence of the URA3 gene from Saccharomyces cerevisiae situated within a multiple cloning site (MCS). pUC19 contains the pMB1 replicon and a gene coding for beta-lactamase for replication and selection in Escherichia coli. In addition to the coding sequence for URA3, the sequences from upstream and downstream of this gene were included for expression of the URA3 gene in yeast. The vector can be used for cloning purposes and can be used as a yeast integration vector.

The DNA encompassing the URA3 coding region along with 250 bp upstream and 150 bp downstream of the URA3 coding region from Saccharomyces cerevisiae CEN.PK 113-7D genomic DNA was amplified with primers oBP438 (SEQ ID NO: 12) containing BamHI, AscI, PmeI, and FseI restriction sites, and oBP439 (SEQ ID NO: 13) containing XbaI, PacI, and NotI restriction sites, using Phusion® High Fidelity PCR Master Mix (New England BioLabs Inc., Ipswich, Mass.). Genomic DNA was prepared using a Gentra® Puregene® Yeast/Bact. kit (Qiagen, Valencia, Calif.). The PCR product and pUC19 (SEQ ID NO: 72) were ligated with T4 DNA ligase after digestion with BamHI and XbaI to create vector pUC19-URA3MCS. The vector was confirmed by PCR and sequencing with primers oBP264 (SEQ ID NO: 10) and oBP265 (SEQ ID NO: 11).

The coding sequence of sadB and PDC5 Fragment B were cloned into pUC19-URA3MCS to create the sadB-BU portion of the PDC5 A-sadB-BUC PCR cassette. The coding sequence of sadB was amplified using pLH468-sadB (SEQ ID NO: 67) as template with primer oBP530 (SEQ ID NO: 50) containing an AscI restriction site, and primer oBP531 (SEQ ID NO: 51) containing a 5′ tail with homology to the 5′ end of PDC5 Fragment B. PDC5 Fragment B was amplified with primer oBP532 (SEQ ID NO: 52) containing a 5′ tail with homology to the 3′ end of sadB, and primer oBP533 (SEQ ID NO: 53) containing a PmeI restriction site. PCR products were purified with a PCR Purification kit, (Qiagen, Valencia, Calif.). sadB-PDC5 Fragment B was created by overlapping PCR by mixing the sadB and PDC5 Fragment B PCR products and amplifying with primers oBP530 (SEQ ID NO: 50) and oBP533 (SEQ ID NO: 53). The resulting PCR product was digested with AscI and PmeI and ligated with T4 DNA ligase into the corresponding sites of pUC19-URA3MCS after digestion with the appropriate enzymes. The resulting plasmid was used as a template for amplification of sadB-Fragment B-Fragment U using primers oBP536 (SEQ ID NO: 54) and oBP546 (SEQ ID NO: 55) containing a 5′ tail with homology to the 5′ end of PDC5 Fragment C. PDC5 Fragment C was amplified with primer oBP547 (SEQ ID NO: 56) containing a 5′ tail with homology to the 3′ end of PDC5 sadB-Fragment B-Fragment U, and primer oBP539 (SEQ ID NO: 57). PCR products were purified with a PCR Purification kit (Qiagen, Valencia, Calif.). PDC5 sadB-Fragment B-Fragment U-Fragment C was created by overlapping PCR by mixing PDC5 sadB-Fragment B-Fragment U and PDC5 Fragment C and amplifying with primers oBP536 (SEQ ID NO: 54) and oBP539 (SEQ ID NO: 57). The resulting PCR product was purified on an agarose gel followed by a Gel Extraction kit (Qiagen, Valencia, Calif.). The PDC5 A-sadB-BUC cassette (SEQ ID NO: 71) was created by amplifying PDC5 sadB-Fragment B-Fragment U-Fragment C with primers oBP542 (SEQ ID NO: 58) containing a 5′ tail with homology to the 50 nucleotides immediately upstream of the native PDC5 coding sequence, and oBP539 (SEQ ID NO: 57). The PCR product was purified with a PCR Purification kit (Qiagen, Valencia, Calif.).

Competent cells of CEN.PK 113-7D Δura3::loxP Δhis3 Δpdc6 Δpdc1::ilvDSm were made and transformed with the PDC5 A-sadB-BUC PCR cassette using a Frozen-EZ Yeast Transformation II™ kit (Zymo Research Corporation, Irvine, Calif.). Transformation mixtures were plated on synthetic complete media lacking uracil supplemented with 1% ethanol (no glucose) at 30° C. Transformants with a pdc5 knockout sadB integration were screened for by PCR with primers oBP540 (SEQ ID NO: 59) and oBP541 (SEQ ID NO: 60) using genomic DNA prepared with a Gentra® Puregene® Yeast/Bact. kit (Qiagen, Valencia, Calif.). The absence of the PDC5 gene from the isolate was demonstrated by a negative PCR result using primers specific for the coding sequence of PDC5, oBP552 (SEQ ID NO: 61) and oBP553 (SEQ ID NO: 62). A correct transformant was selected as strain CEN.PK 113-7D Δura3::loxP Δhis3 Δpdc6 Δpdc1::ilvDSm Δpdc5::sadB-URA3.

CEN.PK 113-7D Δura3::loxP Δhis3 Δpdc6 Δpdc1::ilvDSm Δpdc5::sadB-URA3 was grown overnight in YPE (1% ethanol) and plated on synthetic complete medium supplemented with ethanol (no glucose) and containing 5-fluoro-orotic acid (0.1%) at 30° C. to select for isolates that lost the URA3 marker. The deletion of PDC5, integration of sadB, and marker removal were confirmed by PCR with primers oBP540 (SEQ ID NO: 59) and oBP541 (SEQ ID NO: 60) using genomic DNA prepared with a Gentra® Puregene® Yeast/Bact. kit (Qiagen, Valencia, Calif.). The correct isolate was selected as strain CEN.PK 113-7D Δura3::loxP Δhis3 Δpdc6 Δpdc1::ilvDSm Δpdc5::sadB and designated as BP913.

### GPD2 Deletion

To delete the endogenous GPD2 coding region, a gpd2::loxP-URA3-loxP cassette (SEQ ID NO: 73) was PCR-amplified using loxP-URA3-loxP (SEQ ID NO: 68) as template DNA. loxP-URA3-loxP contains the URA3 marker from (ATCC No. 77107) flanked by loxP recombinase sites. PCR was done using Phusion® DNA polymerase (New England BioLabs Inc., Ipswich, Mass.) and primers LA512 and LA513 (SEQ ID NOs: 8 and 9). The GPD2 portion of each primer was derived from the 5′ region upstream of the GPD2 coding region and 3′ region downstream of the coding region such that integration of the loxP-URA3-loxP marker resulted in replacement of the GPD2 coding region. The PCR product was transformed into BP913 and transformants were selected on synthetic complete media lacking uracil supplemented with 1% ethanol (no glucose). Transformants were screened to verify correct integration by PCR using primers oBP582 and AA270 (SEQ ID NOs: 63 and 64).

The URA3 marker was recycled by transformation with pRS423::PGAL1-cre (SEQ ID NO: 66) and plating on synthetic complete media lacking histidine supplemented with 1% ethanol at 30° C. Transformants were streaked on synthetic complete medium supplemented with 1% ethanol and containing 5-fluoro-orotic acid (0.1%) and incubated at 30° C. to select for isolates that lost the URA3 marker. 5-FOA resistant isolates were grown in YPE (1% ethanol) for removal of the pRS423::PGAL1-cre plasmid. The deletion and marker removal were confirmed by PCR with primers oBP582 (SEQ ID NO: 63) and oBP591 (SEQ ID NO: 65). The correct isolate was selected as strain CEN.PK 113-7D Δura3::loxP Δhis3 Δpdc6 Δpdc1::ilvDSm Δpdc5::sadB Δgpd2::loxP and designated as PNY1503 (BP1064).

BP1064 was transformed with plasmids pYZ090 (SEQ ID NO: 1) and pLH468 (SEQ ID NO: 2) to create strain NGC1-070 (BP1083; PNY1504).

1. A method of producing butanol comprising:
(a) providing a composition comprising (i) a microorganism capable of producing butanol and (ii) an enzyme or combination of enzymes capable of converting a 5-carbon sugar to xylulose or xylulose-5-phosphate;
(b) contacting the composition with a carbon substrate comprising mixed sugars; and
(c) culturing the microorganism under conditions of limited oxygen utilization, whereby butanol is produced.

2. (canceled)

3. (canceled)

4. The method of claim 1, wherein the enzyme or combination of enzymes capable of converting a 5-carbon sugar to xylulose or xylulose-5-phosphate are recombinantly expressed by a microorganism in the composition.

5. The method of claim 4, wherein the enzyme or combination of enzymes capable of converting a 5-carbon sugar to xylulose or xylulose-5-phosphate are recombinantly expressed by the microorganism capable of producing butanol.

6. The method of claim 4, wherein the enzyme or combination of enzymes capable of converting a 5-carbon sugar to xylulose or xylulose-5-phosphate are not produced by the microorganism capable of producing butanol.

7. The method of claim 1, wherein the enzyme or combination of enzymes capable of converting a 5-carbon sugar to xylulose or xylulose-5-phosphate are not produced by a microorganism in the composition.

8. The method of claim 1, wherein the enzyme or combination of enzymes capable of converting a 5-carbon sugar to xylulose or xylulose-5-phosphate is selected from the group consisting of: (i) xylose isomerase; (ii) xylose reductase; (iii) xylitol dehydrogenase; (iv) arabinose isomerase; (v) ribulokinase; (vi) ribulose-phosphate-5-epimerase; (vii) arabinose reductase; (viii) arabitol dehydrogenase; (ix) xylulose reductase; (x) xylulokinase; (xi) aldose reductase; and (xii) combinations thereof.

9. (canceled)

10. (canceled)

11. (canceled)

12. (canceled)

13. (canceled)

14. (canceled)

15. (canceled)

16. (canceled)

17. The method of claim 1, wherein the source of 5-carbon sugars is a source of xylose.

18. The method of claim 1, wherein the source of 5-carbon sugars is a source of arabinose.

19. (canceled)

20. (canceled)

21. The method of claim 1, additionally comprising contacting the composition with a source of 6-carbon sugars.

22. The method of claim 21, wherein 5-carbon and 6-carbon sugars are cumulatively consumed at a rate of at least about 1.5 g/gdcw/h.

23. (canceled)

24. (canceled)

25. The method of claim 21, wherein 5-carbon sugars and 6-carbon sugars are consumed, and the rate of 5-carbon sugar consumption is at least about 1% the rate of 6-carbon sugar consumption.

26. The method of claim 1, wherein the source of 5-carbon sugars is lignocellulosic biomass.

27. (canceled)

28. The method of claim 26, wherein the lignocellulose biomass is pretreated with ammonia.

29. (canceled)

30. (canceled)

31. (canceled)

32. (canceled)

33. (canceled)

34. (canceled)

35. (canceled)

36. (canceled)

37. (canceled)

38. (canceled)

39. The method of claim 1, wherein an inhibitor of respiration is added to the composition.

40. (canceled)

41. The method of claim 1, wherein the composition and/or the source of 5-carbon sugars further comprises a microorganism that is not capable of producing butanol, and the microorganism capable of producing butanol is present at a concentration that is at least equal to 1 g/l.

42. (canceled)

43. The method of claim 1, wherein the specific butanol production is at least about 0.4 g/g/h.

44. (canceled)

45. The method of claim 1, further comprising purifying butanol from the culture.

46. (canceled)

47. A butanol composition obtained by the method of claim 1

48. (canceled)

49. A composition for producing butanol comprising:
(a) a microorganism capable of producing butanol;
(b) an enzyme or combination of enzymes capable of converting a 5-carbon sugar to xylulose or xylulose-5-phosphate;
(c) a source of 5-carbon sugars; and
(d) a fermentation media.

50. The composition of claim 49, wherein the butanol is isobutanol.

51. The composition of claim 49, wherein the microorganism is a yeast.

52. (canceled)

53. (canceled)

54. The composition of claim 49, wherein the source of 5-carbon sugars is present at a concentration of at least about 20 g/L.

55. (canceled)

56. (canceled)

57. The composition of claim 49, wherein the microorganism capable of producing butanol comprises polynucleotides encoding polypeptides that catalyze the conversion of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol.

58. (canceled)

59. (canceled)

60. (canceled)

61. The composition of claim 49, wherein the microorganism capable of producing butanol is Saccharomyces cerevisiae.

62. (canceled)

63. The composition of claim 49, further comprising a microorganism that does not produce butanol.

64. (canceled)

65. The composition of claim 49, wherein the composition is capable of producing butanol and ethanol.

66. The composition of claim 49, wherein the composition is capable of producing butanol at least about 0.4 g/g % theoretical yield.

67. (canceled)

68. (canceled)

69. (canceled)

70. (canceled)

71. (canceled)

72. (canceled)

73. (canceled)

74. (canceled)

75. (canceled)

76. (canceled)
